(19) United States
(12) Patent Application Publication
(10) Pub. No.: US 2010/0160250 A1
(43) Pub. Date: Jun. 24, 2010

(54) METHOD FOR TREATING INFLAMMATORY CONDITIONS

(76) Inventors: James G. Douglass, III, Apex, NC (US); Sammy R. Shaver, Chapel Hill, NC (US); Tomas Navratil, Carrboro, NC (US); José L. Boyer, Chapel Hill, NC (US); Carl A. Samuelson, Durham, NC (US); Jonathan B. DeCamp, Raleigh, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/641,208

(22) Filed: Dec. 17, 2009

Related U.S. Application Data
(60) Provisional application No. 61/138,873, filed on Dec. 18, 2008.

(51) Int. Cl. A61K 31/70; C07H 19/167

(52) U.S. Cl. 514/46; 536/27.7

(57) ABSTRACT

This invention provides a method for inhibiting the release of interleukin-1β in a mammal. This invention also provides a method for preventing or treating pulmonary diseases, ophthalmic diseases, and autoimmune diseases that are associated with inflammation or inflammatory conditions. The invention also provides a method for preventing or treating neurodegenerative diseases, or pain in a mammal. The method comprises administering to a mammal in need thereof a therapeutically effective amount of a mononucleoside compound, which is an antagonist of P2X₇ receptor.

The present application claims the benefit of U.S. Provisional Application 61/138,873, filed Dec. 18, 2008, which is incorporated herein by reference in its entirety.

### TECHNICAL FIELD

This invention provides a method for preventing or treating pulmonary diseases, ophthalmic diseases, and autoimmune diseases that are associated with inflammation or inflammatory conditions. The invention also provides a method for preventing or treating neurodegenerative diseases, or pain in a mammal. More particularly, this invention relates to a method of treating pulmonary inflammation, rheumatoid arthritis, or inflammation bowel disease, using a mononucleoside compound.

### BACKGROUND OF THE INVENTION

### P2X₇ Receptor as a Target

Cell surface receptors for ATP can be divided into metabotropic (P2Y) and ionotropic (P2X) classes. The metabotropic class belongs to the superfamily of G protein-coupled receptors, with seven transmembrane segments. The ionotropic class members (P2X₁-P2X₇) are ligand-gated ion channels, currently, with the exception of P2X₆, these receptors are thought to be multi-subunit proteins with two transmembrane domains per subunit (Buell et al, Europ. J. Neurosci. 8:2221 (1996)). P2X₇ receptors (previously known as P2Z) have been distinguished from other P2 receptors in three primary ways (Buisman et al, Proc. Natl. Acad. Sci. USA 85:7988 (1988); Cockcroft et al, Nature 279:541 (1979); Steinberg et al, J. Biol. Chem. 262:3118 (1987)). First, activation of P2X₇ receptors leads not only to an inward ionic current, but also to cell permeabilization. Second, 3′-O-(4-benzoyl)benzoyl ATP (BZATP) is the most effective agonist, and ATP itself is of rather low potency. Third, the signaling of the P2X₇ receptor appears to be elicited by the fully ionized form of ATP, ATP⁴⁻. Consistent with this observation, a marked inhibition of the P2X₇ is observed in the presence of Mg⁺² ions. (DiVirgilio, Immunol. Today 16:524 (1995)).

P2X₇ receptor is structurally related to other members of the P2X family but it has a longer cytoplasmic C-terminus domain (there is 35-40% amino acid identity in the corresponding region of homology, but the C-terminus is 239 amino acids long in the rP2X7 receptor compared with 27-20 amino acids in the others). The P2X₇ receptor functions both as a channel permeable to small cations and as a cytolytic pore. Brief applications of ATP (1-2 s) transiently open the channel, as is the case of other P2X receptors. However, in P2X₇ receptor expressing cells, ATP also produce a gradual opening of a large pore that allows the passage of molecules up to 800 Da. The unique C-terminal domain of P2X₇ receptor is required for cell permeabilization and the lytic actions of ATP (Suprenant et al, Science 272:735 (1996)).

P2X7 is expressed in cells of the hematopoietic lineage, e.g. macrophages, microglia, mast cells, and lymphocytes (T and B) (Collo, et al. Neuropharmacology, vol. 36, pp 1277, 1997). P2X7 activation has been implicated in giant cell formation, degranulation, cytolytic cell death, CD62L shedding, regulation of cell proliferation, and release of pro-inflammatory cytokines such as IL-1B (Ferrari et al, J immunology, vol 176, p 3877, 2006) and TNFalpha (Hide et al, Journal of Neurochemistry, vol 75, p 965). P2X₇ receptor is also located on antigen-presenting cells (APC), keratinocytes, salivary acinar cells (parotid cells), hepatocytes, mesangial cells, monocytes, fibroblasts, bone marrow cells and neurons. Furthermore, the P2X7 receptor is expressed by the presynaptic terminals in the central and peripheral nervous systems and has been shown to mediate glutamate release in glia

As noted above, P2X₇ activation results in rapid but reversible channel opening permeable to Ca²⁺, Na⁺ and K⁺ which in turn leads to activation of caspase-1 and IL-1β processing and release. IL-1β release is independent of cytolysis, does not require pore formation, and is inhibited by P2X₇ receptor antagonists. P2X₇ receptor activation also leads to the release of MMP-9, TNF-A (REF) and potentially other pro-inflammatory mediators. IL-1β is a potent pro-inflammatory mediator and causes recruitment of neutrophils, macrophages and lymphocytes, pulmonary inflammation, elastin degradation, collagen deposition, lung tissue fibrosis and remodeling, emphysema, mucus cell metaplasia, pro-inflammatory and remodeling mediator release and mucus secretion (Hallegua et al. Ann Rheum Dis 2002, 61:960). ATP has been shown to increase local release and processing of IL-1βfollowing lipopolysaccharide S (LPS) intraperitoneal injections in rats through a P2X₇ receptor mediated mechanism (Griffiths et al., J. Immunology Vol. 154, pages 2821-2828 (1995); Solle et al., J. Biol. Chemistry, Vol. 276, pages 125-132, (2000)). P2X₇ receptor −/− knockout mice fail to secrete IL-1β in response to stimuli that normally elicit IL-1β secretion. P2X₇ receptor −/− knockout mice also show less frequent and less severe arthritis in monoclonal anti-body-induced model of collagen arthritis.

Increased IL-1β is linked to alteration of pain sensitivity in experimental models. IL-1β levels are increased in the nervous system in response to trauma and are associated with enhanced nociceptive signaling. Blocking IL-1β receptor with IL-1 receptor antagonist, a naturally occurring protein antagonist of IL-1β, reduces nociception in models of inflammation and nerve injury induced pain. P2X₇ receptor −/− knockout mice demonstrate reduced sensitivity to pain in complete Freund's adjuvant-induced inflammation and partial injury of the sciatic nerve. An active ongoing research further substantiates the evidence pointing to master mediator and initiator role of IL-1 within the inflammatory response and reiterates the validity of therapeutic approaches targeting IL-1 as well as upstream or downstream modulators of IL-1 transcription, translation, processing, secretion or extracellular signaling via agents such as P2X7 antagonists or IL-1 receptor antagonist, respectively (Mills et al. Nature Medicine 2009, 15(12): 1363; Dinarello et al. Annu. Rev. Immunol. 2009, 27:519-50).

### Asthma

Asthma is a common chronic disorder of the airways characterized by variable and recurring symptoms, reversible airway obstruction, bronchial hyperresponsiveness, and an underlying inflammation. Acute symptoms of asthma include cough, wheezing, shortness of breath and nocturnal awakening. These symptoms usually arise from bronchospasm and require and respond to bronchodilator therapy (see Expert Panel Report 3: Guidelines for the Diagnosis and Management of Asthma, NIH Publication No. 07-4051, Bethesda, Md.: U.S. Department of Health and Human Services; National Institutes of Health; National Heart, Lung, and Blood Institute; National Asthma Education and Prevention Program, (2007) and references therein).

Central to the pathophysiology of asthma is the presence of underlying airway inflammation mediated by the recruitment and activation of multiple cell types including mast cells, eosinophils, T lymphocytes, macrophages, dendritic cells and neutrophils. Type 2 T-helper (Th2) cells appear to play a central role in the activation of the immune cascade that results in inflammation. Th2-derived cytokines include IL-5, which is needed for eosinophil differentiation and survival, and IL-4 which is important for Th2 cell differentiation and with IL-13 is important for IgE formation and leads to overproduction of IgE and eosinophilia. IgE-driven activation of mucosal mast cells releases bronchoconstrictor mediators such as histamine and cysteinyl-leukotrienes as well as inflammatory cytokines. Eosinophils contain inflammatory enzymes, generate leukotrienes, and express a wide variety of pro-inflammatory cytokines. Airway epithelial cells also play a role in the inflammatory process via release of cytokines such as eotaxin that direct and modify the inflammatory response. Acute and chronic inflammation can affect not only the airway caliber and airflow but also can increase the existing bronchial hyperresponsiveness to a variety of stimuli, which enhances susceptibility to bronchospasm.

As a consequence of airway inflammation and the generation of growth factors, the airway smooth muscle cell can undergo proliferation, activation, contraction, and hypertrophy events that can influence airway airflow limitation. In asthma, the dominant physiological event leading to clinical symptoms is airway narrowing and a subsequent interference with airflow. In acute exacerbations of asthma, bronchial smooth muscle contraction (bronchoconstriction) occurs quickly to narrow the airways in response to exposure to a variety of stimuli including allergens or irritants. Allergen-induced acute bronchoconstriction results from an IgE-dependent release of mediators from mast cells that includes histamine, tryptase, leukotrienes, and prostaglandins that directly contract airway smooth muscle. The mechanisms influencing airway hyperresponsiveness are multiple and include inflammation, dysfunctional neuroregulation, and airway remodeling. Airway remodeling involves structural changes including thickening of the sub-basement membrane, subepithelial fibrosis, airway smooth muscle hypertrophy and hyperplasia, blood vessel proliferation and dilation with consequent permanent changes in the airway that increase airflow obstruction and that is not prevented by or fully reversible by current therapies.

Airway epithelium and endothelial cell function are also critically involved in asthma. Upon disease progression, epithelial subbasement membranes thicken with mucus hypersecretion and the formation of inspissated mucus plugs. Changes in endothelial cell integrity lead to edema, another key pathophysiology defining asthmatic change of the airway. These factors serve to further limit airflow and are not directly addressed by current therapies.

Current standard therapies for asthma are a combination of corticosteroids and β2-agonists (anti-inflammatory and bronchodilator drugs). These drugs provide acceptable control of the disease for many asthmatics. However, it is estimated that 5 to 10% of the asthma patients have symptomatic disease despite treatment with this combination of corticosteroids and β2-agonists (Chanez et al., J Allergy Clin Immunol 119:1337-1348 (2007)).

### Chronic Obstructive Pulmonary Disease

Chronic obstructive pulmonary disease (COPD) is the most common chronic lung disease associated with significant morbidity and mortality. In the United States, COPD is the fourth leading cause of death and accounts for more than $30 billion in annual health care costs. An estimated 16 million adults are affected by COPD, and each year ˜120,000 Americans die of the disease. COPD is defined as a chronic disease characterized by airway/alveolar/systemic inflammation, with measured airflow obstruction (FEV₁/FVC<70% and FEV₁<80% predicted) that is partially improved with bronchodilator therapy. The local and systemic release of inflammatory mediators by the lung cells leads to airway disease (chronic obstructive bronchitis) and, in a minority of patients, to destruction of parenchymal tissue (emphysema), both of which can result in the airflow limitation that characterizes COPD (see Doherty D E et al, Clin Cornerstone 6:S5-16 (2004) and MacNee, Clin Ches Med 28:479-513 (2007)). The release of these inflammatory mediators by the lung cells may also exacerbate inflammation in other organ systems, such as that observed in coronary, cerebrovascular, and peripheral vascular conditions.

The chronic inflammation, airway obstruction, and tissue damage that occur in COPD all result from chronic exposure to inhaled toxic substances, primarily cigarette smoke. In response to the chemical insult of inhaled tobacco smoke, inflammatory cells (including macrophages, neutrophils, and T-lymphocytes, primarily CD8 lymphocytes) are activated in the small and large airways as well as in the lung parenchyma. These activated inflammatory cells release a host of cytokines and other mediators (including tumor necrosis factor-α, interleukin [IL]-8, and leukotriene B₄), which can cause damage to lung tissue. The end result of the release of these cytokines and mediators may be the development of chronic inflammation of the airways, mucus gland hypertrophy and goblet-cell hyperplasia with increased mucus secretion, fibrosis and narrowing of smaller airways, destruction of the parenchyma (the connective tissue/cells in the lungs), and changes in the blood vessels that may result in the development of pulmonary hypertension. These pathologic changes manifest themselves as mucus hypersecretion, limited airflow, hyperinflation, and gas exchange abnormalities which are the major physiologic abnormalities that characterize COPD. A loss in the integrity of the lung's connective tissue leads to a decrease of elastic recoil and hyperinflation.

Current therapies to treat COPD include bronchodilators, especially anticholinergic agents, which help to some degree decrease hyperinflation, therefore increasing inspiratory capacity and relieving dyspnea. Although corticosteroids are an effective treatment for most cases of asthma, the inflammatory cells and mediators in COPD are not sensitive to treatment with systemic or inhaled corticosteroids making treatment with these agents of limited usefulness in COPD.

### RSV Infection

Respiratory syncytial virus (RSV) causes acute respiratory tract illness in persons of all ages. RSV is a leading cause of lower respiratory tract infection (LRTI) in children younger than 2 years. It is associated with up to 120,000 pediatric hospitalizations each year, and is increasing in frequency. RSV also is a significant cause of morbidity and mortality from LRTI in elderly patients (Collins et al., J Virol 82:2040-2055 (2008); Peebles et al., Proc Am Thorac Soc 2:110-115 (2005)).

After replicating in the nasopharynx, RSV infects the small bronchiolar epithelium and extends to the type 1 and 2 alveolar pneumocytes in lung. Pathologic findings of RSV include necrosis of epithelial cells, occasional proliferation of the bronchiolar epithelium, infiltrates of monocytes and T cells centered on bronchial and pulmonary arterioles, and neutrophils between the vascular structures and small airways. This leads to airway obstruction, air trapping and increased airway resistance, and also is associated with a finding of neutrophilia in bronchoalveolar lavage. The immune response to RSV, especially cytokine and chemokine release, appears to play a role in the pathogenesis and severity of bronchiolitis. There is a distinct pattern of cytokines and chemokines induced by RSV infection and some have been associated with disease severity. The cytokines IL-8, IL-6, TNF-alpha, and IL-1 beta can be detected in airway secretions of infected children (Smyth et al. Arch Dis Child 76:210 (1997)), and IL-6 levels correlate with severe disease. Chemokines identified in respiratory tract secretions of children include CCL3, CCL2, CCL11 and CCL5, but only the beta-chemokines, particularly MIP-1 alpha, are associated with severe disease (Welliver et al. Pediatr Infect Dis J 21:457 (2002)).

RSV can involve both lower and upper respiratory tract. Severe lower respiratory tract disease can involve bronchiolitis, bronchospasm, pneumonia, and acute respiratory failure in children. Lower respiratory tract involvement usually occurs with primary infection, and may occur in second infections and can cause wheezing, tachypnea and apnea. Repeat RSV infections occur frequently in children and young adults and result in significant upper respiratory tract symptoms. Signs include cough, coryza, rhinorrhea, and conjunctivitis. RSV infection in adults also may cause short-term airway reactivity.

There is no direct treatment for RSV infection and the respiratory complications it causes. The current therapy for RSV is primarily supportive. Bronchodilator therapy in infants with bronchiolitis, largely caused by RSV infection, did not demonstrate benefit in large randomized trials and systematic reviews. Prophylaxis with palivizumab, a humanized monoclonal antibody, has been indicated for a limited fraction of the pediatric patient population.

### Idiopathic Pulmonary Fibrosis

Idiopathic pulmonary fibrosis (IPF) is a chronic, progressive, fibrotic disorder of the lower respiratory tract that typically affects adults beyond the age of 40. IPF is thought to occur as a result of initial injury to the lung by environmental factors such as cigarette smoke leading to recruitment of neutrophils, lymphocytes and macrophages to the lung alveoli. Release of fibrogenic cytokines, such as TGF-β by alveolar epithelial cells, results in fibroblast proliferation, migration, and fibrosis. These fibroblasts not only fill the respiratory space but also secrete collagen and matrix proteins in response to many cytokines leading to parenchymal remodeling (Shimizu et al., Am J Respir Crit Care Med 163:210-217 (2001)). This differentiation of fibroblasts is likely key to the chronic nature of IPF. These events lead to cough and progressive shortness of breath. IPF patients have compromised lung function and have shown restrictive lung volumes and capacities. Although corticosteroids, immunosuppressive agents, neutrophil elastase inhibitor, hepatocyte growth factor, and interferon gamma-1b have been proposed as treatment agents for IPF, no treatment other than lung transplantation is known to prolong survival and IPF remains a fatal disorder with a 3 to 6 yr median range of survival (Khalil et al. CMAJ 171:153-160 (2004)). Thus, the first line of treatment of IPF has not yet been established.

### Acute Respiratory Distress Syndrome (ARDS) and Ventilator Induced Lung Injury (VILI)

Acute respiratory distress syndrome is a critical illness characterized by acute lung injury leading to permeability pulmonary edema and respiratory failure. ARDS respiratory failure can be caused by various acute pulmonary injuries and is characterized by noncardiogenic pulmonary edema, respiratory distress, and hypoxemia. Despite significant advances in critical care management, overall mortality from ARDS ranges from 25 to 58% (Berstan A D et al. Am J Respir Crit Care Med, 165:443, 2002).

More than 60 causes of ARDS have been identified. A few common causes include sepsis, aspiration of gastric contents, primary bacterial or viral pneumonias, direct chest trauma, ventilator-induced lung injury, prolonged or profound shock, burns, fat embolism, near drowning, massive blood transfusion, transfusion-related lung injury (TRALI), cardiopulmonary bypass, pneumonectomy, acute pancreatitis, inhalation of smoke or other toxic gas, and ingestion of certain drugs (Pepe P et al. Am J Surg, 144:124, 1982; Hudson L D, J Respir Crit Care Med, 151:293, 1995; Zaccardelli D S and Pattishall E N, Crit Care Med, 24:247, 1996; Fowler A et al. Ann Intern Med, 98:593, 1983).

ARDS is described as a “syndrome of acute and persistent inflammation with increased vascular permeability associated with a constellation of clinical, radiological and physiological abnormalities” (Bernard G et al. Am J Respir Crit Care Med, 149:818, 1994; Artigas A et al. Am J Respir Crit Care Med, 157:1332, 1998). The hallmark of ARDS is deterioration in blood oxygenation and respiratory system compliance as a consequence of permeability edema. Whereas a variety of different insults may lead to ARDS, a common pathway probably results in the lung damage and/or failure, leukocyte activation within the lung, along with the release of oxygen free radicals, arachidonic acid metabolites, and inflammatory mediators, resulting in an increase in alveolocapillary membrane permeability. With the loss of this macromolecular barrier, alveoli are flooded with serum proteins, which impair the function of pulmonary surfactant (Said et al. J. Clin. Invest. 44: 458-464; Holm et al. J. Appl. Physio. 63: 1434-1442, 1987). This creates hydrostatic forces that further exacerbate the condition (Jefferies et al., J. Appl. Physio. 64: 5620-5628, 1988), leading to alveolar edema and a concomitant deterioration in gas exchange and lung compliance.

Mechanical ventilation is a common and generally effective means of treating a failing lung. Unfortunately, positive-pressure mechanical support can create or contribute to lung injury (ventilator-induced lung injury, VILI). Mechanical ventilators applying high volumes and pressures can lead to an influx of fluid into the lung. In addition to edema, the injured or ruptured cells trigger a cascade of cellular and biochemical events leading to the inflammation in the lung. Pulmonary sheer stress can develop due to the increased volume as well as due to atelectasis. VILI is also believed to provoke distal airway and alveolar cell inflammation by increasing the production of proinflammatory cytokines. In light of the fact that more than 280,000 Americans are at risk for VILI each year, and mechanical ventilation support and associated intensive care expenditures are estimated in the billions of dollars, VILI is a major public health concern (WO/2007/109582).

### Cystic Fibrosis (CF)

CF is the most common, life threatening, recessively inherited disease of Caucasian populations, with a carrier rate of 1 in 25 and an incidence of 1 in 2,500 live births. CF is a multisystem disease affecting the digestive system, sweat glands, and the reproductive tract, but progressive lung disease continues to be the major cause of morbidity and mortality (Ratjen, F. and Doring, G. Lancet 361:681, 2003). CF patients have abnormal transport of chloride and sodium across the respiratory epithelium, resulting in thickened, viscous airway secretions (Rowe S M et al. N Engl J Med; 352:1992, 2005). Patients develop chronic infection of the respiratory tract with a characteristic array of bacterial flora (Gibson, R L et al. Am J Respir Crit Care Med 168:918, 2003), leading to progressive respiratory insufficiency and eventual respiratory failure. CF is caused by mutations in a single large gene on chromosome 7 that encodes the cystic fibrosis transmembrane conductance regulator (CFTR) protein (Rommens J M et al. Science; 245:1059, 1989; Collins F S. Science; 256:774, 1992; Drumm, M L et al. Mol Genet Med; 3:33, 1993). CFTR has been shown to function as a regulated chloride channel, which in turn may regulate the activity of other chloride and sodium channels at the cell surface (Boucher R C. Am J Respir Crit Care Med. 150:271-281, 1994). Defective CFTR results in abnormal ion transport and airway surface liquid volume with alterations in the rheology of airway secretions, which become thick and difficult to clear (Wine J J. J Clin Invest; 103:309, 1999). These changes result in reduced mucociliary clearance and a propensity for chronic infection of the respiratory tract with resulting inflammation, progressive airway damage, bronchiectasis, progressive respiratory failure, and death (Mickle J E and Cutting G R. Clinics in Chest Med. 19 (3):443-458, 1998).

Respiratory symptoms of CF usually begin early in life (Ratjen, F and Doring, G. Lancet 361:681, 2003). Respiratory manifestations include recurrent, progressively more persistent cough becoming productive, chronic infection (particularly Pseudomonas aeruginosa), and inflammation leading to progressive tissue damage in the airways. Once infection is established, neutrophils are unable to control the bacteria, even though there is massive infiltration of these inflammatory cells into the lung tissue. Recruited neutrophils subsequently release inflammatory cytokines, reactive oxygen species, and elastase, the latter of which overwhelms the antiproteases of the lung and contributes to progressive destruction of the airway walls. In addition, large amounts of DNA and cytosol matrix proteins are released by degranulating neutrophils, contributing to the increased viscosity of the airway mucus (Davis, P B. Pathophysiology of the lung disease in cystic fibrosis. In: Cystic Fibrosis, Davis, P B (Ed), Marcel Dekker, New York 1993. p. 193). Toxic metabolites released by P. aeruginosa increase the rate of neutrophil apoptosis and decreased removal of apoptotic cells by pulmonary macrophages (Bianchi S M et al. Am J Respir Crit Care Med 177:35-43, 2008), contributing to the accumulation of DNA, protein, and cellular debris in the airway and exacerbating inflammation. Lung damage ultimately advances to the stage of irreversible bronchiectasis (dilated, collapsible airways), leading to progressive air and mucus trapping and ultimate respiratory failure. Other late complications include spontaneous pneumothorax (collapsed lung) and hemoptysis (coughing up blood), which may be massive (Flume P A et al. Chest; 128:720, 2005; Flume P A et al. Chest 128:729, 2005). Terminal findings often include severely congested parenchyma, with grossly purulent secretions in dilated airways. The airway epithelium is hyperplastic, often with areas of erosion and squamous metaplasia. Plugs of mucoid material and inflammatory cells are often present in the airway lumen. Submucosal gland hypertrophy and hyperplasia of airway smooth muscle may also be present (Hays S R et al. Thorax 60:226, 2005.)

Airway hyperreactivity is a common finding in CF patients (Hiatt P et al. Am Rev Respir Dis 137:119, 1988). Many CF patients continue to demonstrate bronchial hyperresponsiveness into adolescence and adulthood, with positive correlations between the degree of airway reactivity and the overall severity of lung disease. The response to bronchodilators does not always persist with increasing age, and some patients demonstrate worsening of expiratory airflow in response to treatment with beta-adrenergic reagents (Gibson, R L et al. Am J Respir Crit Care Med 168:918, 2003). This phenomenon may occur when progressive airway damage leads to a loss of cartilaginous support, resulting in an increased reliance on muscle tone for maintenance of airway patency. Muscle relaxation in this setting can cause collapse of such “floppy” airways, leading to increased airflow obstruction.

The chest radiography may appear normal for an extended period in patients with mild lung disease. As the disease progresses, hyperinflation becomes persistent, and interstitial markings become more prominent. Increasing hyperinflation leads to progressive flattening of the diaphragms, a prominent retrosternal space, and kyphosis (curvature of upper spine) in late stages of disease. Thin-walled cysts may appear to extend to the lung surface, and pneumothorax is observed with increasing frequency in older patients. Computed tomography (CT) of the chest may be helpful in defining the extent of bronchiectasis in some patients (de Jong, P A et al. Radiology 231:434, 2004.]. This is of particular interest in patients who have focal areas of advanced disease, which may sometimes be amenable to surgical resection.

Changes in pulmonary function may be identifiable from a very early age, even before clinical signs of disease are apparent (Long F R et al. J Pediatr 144:154, 2004; Castile R G et al. Pediatr Pulmonol 37:461, 2004). Over time, the majority of CF patients develop an obstructive pattern on pulmonary function testing (PFT). Increases in the ratio of residual volume to total lung capacity (RV/TLC) and decreases in the forced expiratory flow at 25 to 75 percent of lung volume (FEF₂₅₋₇₅) provide the most sensitive measures of early airway obstruction. As disease progresses, the forced expiratory volume in one second (FEV₁) and the ratio of FEV₁ to forced vital capacity (FEV₁/FVC) decline (Davis, P B. Pathophysiology of the lung disease in cystic fibrosis. In: Cystic Fibrosis, Davis, P B (Ed), Marcel Dekker, New York 1993. p. 193). The FEV₁ is correlated with subsequent survival in CF patients. An FEV₁ persistently lower than 30 percent of predicted may be a useful indicator of the need for transplant evaluation in patients who are considered appropriate candidates for that procedure (Kerem E et al. N Engl J Med; 326:1187, 1992). Lung volumes demonstrate increases in total lung capacity (TLC) and residual volume (RV) as hyperinflation progresses. Despite aggressive therapy, baseline pulmonary function gradually decreases as patients get older.

As bronchiectasis and airway obstruction become pronounced, ventilation-perfusion mismatch leads to hypoxemia. This may initially occur only during sleep or exercise, but patients with advanced disease often require continuous oxygen supplementation. Hypercapnia occurs relatively late in the course of CF lung disease. Chronic hypoxemia and hypercapnia may lead to muscular hypertrophy of the pulmonary vasculature, pulmonary hypertension, right ventricular hypertrophy, and eventually cor pulmonale with right heart failure (Eckles M and Anderson P. Semin Respir Crit Care Med 24:323-30, 2003).

Therapeutic intervention for cystic fibrosis includes inhaled and oral antibiotics (tobramycin, azithromycin), bronchodilators (β-adrenergic agonists), DNase I (dornase alpha), hypertonic saline, chest physiotherapy, anti-inflammatory agents (azithromycin, ibuprofen, glucocorticoids), and lung transplantation. Although improved treatment of lung disease has increased survival, the median age for survival is still only 35 years (Cystic Fibrosis Foundation Patient Registry Annual Data Report, 2004), and patients continue to have significant morbidity, including hospitalizations (Ramsey B W. N Engl J. Med. 335 (3):179-188, 1996).

### Bronchiectasis

Bronchiectasis is currently defined as the irreversible and sometimes progressive dilatation and destruction of the bronchial wall caused by a vicious pathogenic cycle of impaired local defense mechanisms, infection, and airway inflammation (Garcia, Arch Bronconeumol, 41 (8):407-9, 2005). Bronchiectasis is a syndrome of chronic cough and daily viscid sputum production associated with airway dilatation and bronchial wall thickening. Hemoptysis can also occur. Multiple conditions are associated with the development of bronchiectasis, but all require an infectious insult plus impairment of drainage, airway obstruction, and/or a defect in host defense (Barker, A. F. Clinical manifestations and diagnosis of bronchiectasis. In: UpToDate, King T E (Ed), UpToDate, Wellesley, Mass., 2008).

All types of bronchiectasis are characterized by predominately neutrophilic and mononuclear inflammation with scores of cellular mediators that modulate both acute and chronic inflammatory response and perpetuate the bronchial lesion (Garcia, Arch Bronconeumol, 41 (8):407-9, 2005) The ensuing host response, immune effector cells, neutrophilic proteases, reactive oxygen intermediates (eg, hydrogen peroxide [H₂O₂]), and inflammatory cytokines, causes transmural inflammation, mucosal edema, cratering, ulceration, and neovascularization in the airways. The result is permanent abnormal dilatation and destruction of the major bronchi and bronchiole walls. Recurrent infection is common, which can lead to further scarring, obstruction, and distortion of the airways, as well as temporary or permanent damage to the lung parenchyma (Barker, A. F. Clinical manifestations and diagnosis of bronchiectasis. In: UpToDate, King T E (Ed), UpToDate, Wellesley, Mass., 2008). The characteristic clinical picture is chronic purulent sputum, functional impairment in the form of air flow obstruction, multiple exacerbations of an infectious type that occasionally involve atypical microorganisms, and dyspnea in advanced stages of the disease—all of which cause progressive deterioration of the patient's quality of life (Garcia, Arch Bronconeumol, 41 (8):407-9, 2005). Mortality is difficult to estimate given the difficulty in identifying prevalence and the lack of definitive studies. One study from Finland identified 842 patients aged 35-74 years with bronchiectasis and followed them for 8-13 years. These patients were also compared with asthma and COPD controls. The mortality was not found to be significantly different among the 3 groups (bronchiectasis, asthma, COPD) with mortality rates of 28%, 20%, and 38% respectively. Currently, mortality is more often related to progressive respiratory failure and cor pulmonale than to uncontrolled infection. Life-threatening hemoptysis may also occur but is uncommon (Emmons Bronchiectasis. In: WebMD Hollingsworth, H M (Ed) 2008). Bronchiectasis is the prototypical disease for which secretion loosening or thinning combined with enhanced removal techniques should be salutary, although large population and long-term studies of efficacy are lacking. This is particularly important as tenacious secretions and mucous plugs are frequently present. Potential approaches include hydration, nebulization with saline solutions and mucolytic agents, mechanical techniques, bronchodilators, and anti-inflammatory therapy. (Barker, A. F. Treatment of bronchiectasis. In: UpToDate, King T E (Ed), UpToDate, Wellesley, Mass., 2008.) Treatment of bronchiectasis is aimed at controlling infection and improving bronchial hygiene. Since infection plays a major role in causing and perpetuating bronchiectasis, reducing the microbial load and attendant mediators is a cornerstone of therapy (Barker, A. F. Treatment of bronchiectasis. In: UpToDate, King T E (Ed), UpToDate, Wellesley, Mass., 2008).

Treatment strategies including daily oral antibiotic treatment, daily or three times weekly use of a macrolide antibiotic treatment, aerosolization of an antibiotic, and intermittent intravenous antibiotics have not been established in long-term studies (Barker, A. F,). Several antibiotic treatment strategies are expensive and require extra equipment and personnel and only target part of the pathophysiology of the disease. Other treatments include physiotherapy, hydration with oral liquids and nebulization with hyperosmolar or mucolytic agents, bronchodilators, anti-inflammatory medications such as corticosteroids, and surgery. (Barker, A. F.) Thus, the treatments for bronchiectasis are limited in their ability to affect key pathophysiologies of the disease.

### Alpha-1-Antitrypsin Deficiency (AATD)

AATD is a common inherited genetic disorder which severely affects up to 100,000 people in the US alone. (Campos, M A et al. Chest, 128:1179, 2005). An important physiological role for alpha-1-antitrypsin (AAT) is to protect lung elastin from degradation by serine proteases such as neutrophil elastase, which is repeatedly produced by lung tissues as a normal immune response to inhaled airborne pathogens. Low levels of AAT and/or secretion of defective AAT can lead to an imbalance between antiproteases and their target serine proteases, leading to tissue damage by these potent degrading enzymes (Koehlein, T et al. Am J. Med., 121:3-9, 2008).

A further aspect of the secretion of defective protein is the loss of the anti-inflammatory properties exerted by the normal protein. AAT is mainly produced in the hepatocytes, with the most common inherited AAT defect giving rise to an accumulation of abnormal protein in these cells, often resulting in cell damage (Lomas, D A, et al. Nature, 357:605, 1992). In the lung, the alveoli show low levels of functional AAT, often leading to an imbalance between antiprotease and protease, and consequential tissue destruction. While the correlation between the severity of the protein deficiency and resultant disease is somewhat variable (Silverman, E K et al. Ann Intern Med, 111:982, 1989), AATD is associated with increased risk for COPD, emphysema, asthma, chronic bronchitis, and brochiectasis in the lung, as well as cirrhosis, hepatitis, hepatocarcinoma or liver failure.

A major risk factor for COPD and emphysema in AATD patients is smoking, thus a smoking cessation program is a critical first-line defense against the progression of disease. Current available therapies for COPD and emphysema include use of long acting beta-agonists and anticholinergics to promote bronchorelaxation, treatment with steroids to reduce inflammation, or supplementation of AAT levels with AAT isolated from the pooled blood of human donors. (Koehlein, T et al. Am J Med., 121:3-9, 2008). A recombinant form of AAT is not yet available for clinical use (Trexler, M M, et al. Biotechnol Prog, 18:501, 2002). However, as none of these therapies are particularly effective, there is an unmet medical need for improved drugs for the treatment of AATD induced lung disease.

### Rhinitis

Rhinitis is irritation and inflammation of the mucosal lining of the nose, which may be caused by allergies or other factors such as cigarette smoke, changes in temperatures, and exercise and stress. The resulting irritation and inflammation generate excessive amounts of mucus producing a runny nose, nasal congestion, and post-nasal drip. Rhinitis is a global health concern and is often combined with asthma in determining morbidity due to respiratory diseases. It is a complex disease affecting approximately 20% of the population. Rhinitis occurs in different types: allergic or atopic rhinitis including seasonal and perennial forms. The mechanism of perennial rhinitis with non-allergic triggers is not well understood. It is an allergy-like condition but not triggered by allergens. (Braunstahl et al. Current Opinion in Pulmonary Medicine 2003, 9:46-51). Idiopathic non-allergic rhinitis or vasomotor rhinitis is characterize by nasal congestion and post nasal drip in responses to temperature and humidity changes, smoke, odors, and emotional upsets. In general rhinitis is characterized by a symptoms complex that consists of any combination of the following: sneezing, nasal congestion, nasal itching and irritation, sneezing and watery rhinorrhea, frequently accompanied by nasal congestion. Perennial allergic rhinitis clinical symptoms are similar, except that nasal blockage may be more pronounced. Each type of allergic rhinitis may cause additional symptoms such as itching of the throat and/or eyes, excessive tearing, and edema around the eyes. These symptoms may vary in intensity from the nuisance level to debilitating. Other types of rhinitis present the same symptoms (Kim et al. Current Opinions in Otolaryngology & Head and Neck Surgery 2007, 15: 268-273).

### Rhinosinusitis

Rhinosinusitis, an inflammation of the sinus cavity, is the most commonly diagnosed chronic illness in the United States. The name of the disease “rhinosinusitis” is preferred over sinusitis as the inflammation of the sinuses rarely occurs without inflammation of the nasal mucosal at the same time. The disease affects over thirty million people in the United States alone. The treatments for rhinosinusitis are costly, exceeding $200 million per year. This illness is detrimental to both the overall quality of life and economic welfare of sufferers. Currently there is no universally accepted treatment for rhinosinusitis; therefore a need to identify new molecular pathways targeting the disease exists.

Sinusitis is the inflammation of the mucus membranes involving the paranasal sinuses, nasal cavity, and underlying bone. A leading theory suggests that exposure to allergens induces inflammation in the small channels of the ostiomeatal complex (OMC), which results in mucosal edema and ultimately impaired mucociliary clearance of the sinus ostia leading to blockage. As a result the trapped mucus becomes a breeding ground for bacteria and other microorganisms which can lead to infection. Common symptoms include pain varying from forehead to teeth, cheeks, ears, and neck, nasal drainage or postnasal drip and decreased sense of smell (Metson, R. et al. Chronic rhinosinusitis, In: UpToDate, Calderwood, S B (Ed), UpToDate, Wellesley, Mass., 2008).

Depending upon the durations of symptoms, rhinosinusitis may be classified as acute, sub acute, or chronic. Chronic sinusitis has long-term effects that could last over twelve weeks and accounts for >90% of all cases of rhinosinusitis The effects of chronic rhinosinusitis are debilitating even when compared to other chronic illnesses such as heart failure or pulmonary disease because it has potential to cause physical and physiological impairment (Metson, R. et al. Chronic rhinosinusitis, In: UpToDate, Calderwood, S B (Ed), UpToDate, Wellesley, Mass., 2008).

### Bronchiolitis

Bronchiolitis is an acute lower respiratory tract infection which typically resolves on its own in previously well children and high risk adults. Although 70-80% of cases are caused by respiratory syncytial virus (RSV), other pathogens such as parainfluenza virus types 1, 2, and 3, and the adenovirus are all known causes. Far less commonly, bronchiolitis can be associated with human metapneumovirus, sometimes concurrently with RSV infection, and chlamydial pneumonia. While the illness is typically self-limiting, 2-3% of all infants are admitted to the hospital for bronchiolitis each winter, and of these admitted children, 2-5% will require mechanical ventilation (Gunn, V. Bronchiolitis. In: FirstConsult, MDConsult, 2007). Increased levels of IL-1B have been described in clinical manifestations as well as in vivo models of infections with agents causing bronchiolitis (Devergne 1991 Eur Cytokine Netw., 2 (3):183; Guerrero-Plate et al. 2005, Journal of Virology, 79 (23): 14992; Ginsberg et al. 1991, PNAS, 88 (5):1651).

Clinical symptoms of bronchiolitis are typically preceded by a prodrome suggesting upper respiratory infection. Patients present with irritability, restlessness, fever, cough, coryza, labored breathing, and tachypnea. Children are often found hypoxemic and can be accompanied by hypercapnia and acidosis. Upon infection, bacterial- or viral-induced inflammation occurs and causes hypersecretion of mucus and submucosal edema. This excess mucus and swelling creates plugs that obstruct the bronchioles which leads to hyperinflation or lung collapse in distal tissues. This explains the increased severity of bronchiolitis in pre-term infants, whose small bronchioles cannot tolerate any thickening of the mucosal wall (Gunn, V. Bronchiolitis. In: FirstConsult, MDConsult, 2007).

Treatment is directed towards managing symptoms on an outpatient basis, although infants in respiratory distress should be hospitalized for supportive care. Beta agonist therapy appears to have little place in the treatment of bronchiolitis, but some infants predisposed to bronchospasm may benefit from its use. Trials conducted with corticosteroids have failed to show any effect, whether harmful or beneficial towards the course of bronchiolitis (Dipiro, J T. Bronchiolitis. In: Pharmacotherapy: A Pathophysiologic Approach, 7^{th} ed, 2008. pg. 1767-1768). Infants with acute disease are at most risk from further respiratory distress during the first 48 to 72 hours from the onset of cough and dyspnea, in which they can become apneic and experience respiratory acidosis. For outpatient children, the median duration of symptoms is about 12 days, but can be lengthened and present more severely in those with conditions such as congenital heart disease, immunodeficiency, or bronchopulmonary dysplasia (Watts, K D. Wheezing in Infants: Bronchiolitis. In: Nelson Textbook of Pediatrics, 18^{th} ed, 2007. Ch. 388).

### Pneumonia

Pneumonia is the most common cause of death from infection in the United States, and costs the healthcare system over $20 billion. Community-acquired pneumonia (CAP) can be bacterial, viral, fungal, or parasitic in origin. The leading causative agent in community-acquired pneumonia is S. pneumoniae, accounting for up to 75% of acute cases. Other common pathogens include M pneumoniae, Legionella, C. pneumoniae, and H. influenzae in the community, and gram-negative bacilli and S. aureus in the hospital setting. Pneumonia may be caused by a wide array of organisms, but presenting clinical appearances are generally similar. Pneumonia is typically characterized by an abrupt onset of fever, chills, and dyspnea, a productive cough, rust-colored sputum or hemoptysis, and pleuritic chest pain. Older populations usually exhibit fewer symptoms (Dipirio, Joseph et al. Pharmacotherapy: A Pathophysiologic Approach 7^{th} ed., pg. 1772-1777). Increased levels of IL-1B in clinical manifestations of pneumonia as well as in vivo models of infections with agents causing pneumonia have been described (Lieberman et al. Infection 1997, 25 (2):90; Pietsch et al Microbiology 1994, 140 (Pt 8): 2043; Asai et al. Vet Immunol Immunophathol. 1993, 38 (3-4): 253; Yang et al. Microb Pathog. 2003, 34 (1):17).

Pneumococcal pneumonia infection occurs when the inhaled or aspirated pneumococci overtake the host defense system in the respiratory tract. Pneumococcus is commonly acquired in the nasopharynx and as many as one-half of all individuals asymptomatically carry the bacterium at any point in time. As infection progresses, bacteria pass through the alveoli creating inflammation and a foamy, serous, blood-tinged fluid within the alveolar spaces. Upon broncheoalveolar lavage, high amounts of tumor necrosis factor, IL-6, and NO can be detected (Marrie, Thomas et al. Pneumococcal pneumonia in adults In: UpToDate, Wellesley, Mass., 2009).

Pneumonia may cause mild disease, but severe disease can result in the very young, the elderly, and the chronically ill. Clinical failure can occur in up to 31% of patients with severe CAP, significantly increasing the risk of complications, days in the hospital, and death. It is estimated that over 80% of the causes of clinical failure in those patients that are hospitalized with CAP are directly related to pulmonary infection and the resulting systemic inflammatory response. These failures typically occur during the first 72 hours after admission and are most often related to sepsis. Among these patients with severe sepsis, about 90% of them had received empiric antibiotic therapy in compliance with the ATS/IDSA national guidelines, and no differences were found in microbiological etiologies between patients with failure and those without. Clinical failure can also result from extrapulmonary events associated with the inflammatory response and increased proinflammatory cytokines. Musher et al have established the association between cardiac events and inflammation from respiratory infection (Aliberti et al. Chest 134:5 (2008)).

Current treatment aims to eradicate the offending organism in bacterial pneumonia by selecting an appropriate antibiotic. Most cases of viral pneumonia are self-limiting. Antimicrobial therapy is empiric and based on the patient's probable or documented microbiology, side effects, age, comorbidities, and cost. Knowledge of antibiotic sensitivity patterns in each community is paramount due to ever-increasing bacterial resistance to antibiotics (Dipirio, Joseph et al. Pharmacotherapy: A Pathophysiologic Approach 7^{th} ed., pg. 1772-1777).

### Primary Ciliary Dyskinesia

PCD is a genetic disease that affects approximately one in 10,000 to 30,000 individuals worldwide (PCD coverage in Up-to-Date database). It is probably the 3rd most common form of inherited chronic airway disease of Caucasian children, after cystic fibrosis and genetic immunodeficiency states. (Haddad G, Kashgarin M. 2007. Primary Ciliary Dyskinesia (Immotile Cilia Syndrome Chapter 401. In: Kliegman R M, Behrman R E, et al. Nelson Textbook of Peidiatrics 18^{th} ed. Elsevier Health Sciences Gabriel G. Haddad, Michael Kashgarian)

PCD is an autosomal recessive disorder characterized by the impairment of mucociliary clearance (MCC) (Bush A, Chodhari R, Collins N, et al. 2007. Primary cilary Dyskinesia: current state of the art. Arch Dis Child; 92: 1136-1140). The underlying cause of PCD is a genetic mutation in any of the cilia polypeptides leading to structural defects of the cilia. The structural defects result in reduction or absence of ciliary function termed as ciliary immotility, ciliary dyskinesia, or ciliary aplasia. Abnormal beating or immotility of cilia leads to defective mucociliary clearance of airway secretions.

Thickened viscous airway secretions provide an ideal environment to a variety of pathogenic organisms in the lungs of PCD patients. The most common infections are Haemophilus influenzae, Streptococcus pneumoniae, Staphylococcus aureus, and less commonly, Pseudomonas aeruginosa. Recurrent infections of the upper and lower respiratory tract and inflammation lead to progressive, irreversible injury to the airway walls in PCD patients (PCD coverage in Up-to-Date database).

PCD is characterized by an influx of neutrophils into the lung tissue leading to airway inflammation. Neutrophils subsequently release cytokines, reactive oxygen species, and elastase, the latter of which overwhelms the antiproteases of the lung and contributes to destruction of the airway walls. Lung damage ultimately advances to the stage of irreversible bronchiectasis, leading to progressive air and mucus trapping and ultimate respiratory failure (Bush A, Chodhari R, Collins N, et al. 2007. Primary cilary Dyskinesia: current state of the art. Arch Dis Child; 92: 1136-1140).

PCD patients differ in the cilia components that may be missing or defective thus there is a wide variety of clinical symptoms displayed by PCD patients. (1) Common clinical manifestations of PCD include chronic cough, chronic rhinitis, and chronic sinusitis, a higher incidence of respiratory infections and bronchiectasis in all adults and 50% children.

There are currently no approved therapies for the treatment of PCD. Clinical recommendations are based on cystic fibrosis treatments. Mucociliary clearance and antibiotics to treat respiratory infections are both common practices in PCD patient treatment (PCD coverage in Up-to-Date database).

### OB/BOOP Due to Lung Transplantation and HSCT

Obliterative bronchiolitis (OB) is characterised by the onset of new air flow obstruction due to functional obstruction of the bronchioles. OB is a common late noninfectious pulmonary complication following both lung transplantation and allogeneic haematopoietic stem cell transplantation (HSCT) with an incidence of 50-60% in patients who survive for 5 years after lung transplantation and 0-48% following HSCT. OB accounts for more than 30% of all deaths occurring after the third postoperative year for lung transplant patients. The mortality rate in patients with OB following HSCT varies from 14-100%, with a median of 65%. Graft versus host disease is an established risk factor for OB after lung transplantation and HSCT. The histopathologic features of OB suggest that injury and inflammation of epithelial cells and subepithelial structures of small airways lead to excessive fibroproliferation, seemingly due to ineffective epithelial regeneration and aberrant tissue repair. The respiratory symptoms of OB include dry cough, dyspnea, and wheezing. Lung biopsies show small airway involvement with fibrinous obliteration of the lumen. BAL shows neutrophilic and/or lymphocytic inflammation. Despite treatment with corticosteroids and immunosuppression, improvement in lung function is noted in only 8% to 20% of patients with OB. Most patients with OB progress to respiratory failure, and some patients develop bronchiectasis with frequent bacterial exacerbations (Afessa B, Bone Marrow Transplantation 28: 524-434, 2001; Nicod L P, Proc Am Thorac Soc 3: 444-449, 2006; Estenne M, Am J Respir Crit Care Med 166: 440-444, 2002).

Bronchiolitis obliterans organizing pneumonia (BOOP) is a complication of both lung transplantation and HSCT and is defined by the patchy distribution of plugs of granulation tissue that fill the lumens of the distal airways, extending into the alveolar ducts and alveolar sacs in association with chronic interstitial inflammation. Organizing pneumonia results from alveolar epithelial injury with subsequent intra-alveolar fibrosis, angiogenesis and inflammation. Clinically, patients present with fever, cough, dyspnea, and crackles on physical examination with onset between 1 and 13 months following HSCT. The clinical spectrum of BOOP ranges from a mild illness to respiratory failure and death. BOOP usually responds well to corticosteroid treatment, however, frequent relapse occurs and new therapeutic options are needed to treat BOOP. (Cordier et al, Eur Resp J, 28:422-446, 2006; Freudenberger T D et al. Blood, 102:3822-3828, 2003; Travis W D et al. Am J Respir Crit Care Med 165: 277-304, 2002).

The therapeutic options for BO/BOOP include corticosteroids and immunosuppressive agents. However, these treatments are often of limited efficacy and new treatment options are needed to address BO/BOOP following lung transplantation and HSCT.

### Non-IPF Idiopathic Interstitial Pneumonia

The idiopathic interstitial pneumonias (IIPs) are a group of interstial lung diseases (ILD, also know as diffuse parenchymal lung disease or DPLD) that result from damage to the lung parenchyma by varying patterns of inflammation and fibrosis. The interstitium includes the space between the epithelial and endothelial basement membranes and it is the primary site of injury in the IIPs. However, these disorders frequently affect not only the interstitium, but also the airspaces, peripheral airways, and vessels along with their respective epithelial and endothelial linings. The IIPs described comprise a number of clinicopathologic entities, which are sufficiently different from one another to be designated as separate disease entities. The idiopathic interstitial pneumonias include the entities of idiopathic pulmonary fibrosis (IPF), nonspecific interstitial pneumonia (NSIP), cryptogenic organizing pneumonia (COP), acute interstitial pneumonia (AIP), respiratory bronchiolitis-associated interstitial lung disease (RB-ILD), desquamative interstitial pneumonia (DIP), and lymphocytic interstitial pneumonia (LIP). Several clinical findings common to the IIPs are exertional dyspnea or cough, bilateral diffuse interstitial infiltrates on chest radiographs, physiological and gas exchange abnormalities including a decreased carbon monoxide diffusion capacity (DLCO) and an abnormal alveolar-arteriolar PO₂ difference, and histopathologic abnormalities of the pulmonary parenchyma that are characterized by varying marked inflammation, fibrosis and remodeling (Raghu G et al. Clin Chest Med 25:409-419, 2004; Travis W D et al. Am J Respir Crit Care Med 165: 277-304, 2002). The clinical prognosis of these diseases ranges from mild illness to respiratory failure and death. Therapies for the IIPs include corticosteroids and immunosuppressive agents but current treatments are variably effective and new treatment options are needed to treat these diseases.

ILD Other than IPF, Non-IPF IIP, and OB/BOOP

Interstitial Lung Disease (ILD), also known as diffuse parenchymal lung disease (DPLD), represent a variety of disorders that lead to diffuse remodeling, architectural damage to normal lung tissue and inflammation that lead to progressive loss of lung function. In addition to the inflammation and fibrosis that is often seen in the lung parenchyma in ILD, the airways and the vasculature may also be prominently affected. The ILDs can be classified into 7 main groups: iatrogenic or drug-induced; occupational or environmental; granulomatous diseases including pulmonary sarcoidosis collagen-vascular disease; unique entities such as alveolar proteinosis, Langerhans cell granulomatosis, and lymphangioleiomyomatosis; idiopathic interstitial pneumonias including interstitial pulmonary fibrosis (IPF); and inherited disorders such as tuberous sclerosis, neurofibromatosis, metabolic storage disorders and Hermansky-Pudlak syndrome. The most prominent forms of ILD are IPF and pulmonary sarcoidosis. Several g28 clinical findings are common to the ILDs: exertional dyspnea or cough; bilateral diffuse interstitial infiltrates on chest radiographs; physiological and gas exchange abnormalities including a decreased carbon monoxide diffusion capacity (DLCO) and an abnormal alveolar-arteriolar PO₂ difference; and histopathologic abnormalities of the pulmonary parenchyma that are characterized by varying degrees of inflammation, fibrosis and remodeling. The incidence of ILD is estimated to be 31.5 per 100,000/yr in males and 26.1 per 100,000/yr in females and the clinical prognosis of these diseases range from mild illness to respiratory failure and death (Raghu G et al. Clin Chest Med 25:409-419, 2004). The standard therapies for ILD include corticosteroids and immunosuppressive agents but current treatments are variably effective depending on the specific disease entity being treated and new treatment options that suppress inflammation and prevent fibroblast and myofibrobalst proliferation are needed to treat these diseases (Kim et al. Ther Adv Respir Dis 2:319-338, 2008).

### Rheumatoid Arthritis

Rheumatoid arthritis (RA) is classified as an inflammatory disorder resulting from acute and chronic inflammation in the synovium that is associated with a proliferative and destructive process in the joint tissue (Harris, E D. Overview of the management of rheumatoid arthritis. In: UpToDate, Schur, P H (Ed), UpToDate, Wellesley, Mass., 2008). One of the earliest pathogenic responses in RA is the generation of new blood vessels, angiogenesis, which is recognized as being fundamental to establishing and perpetuation of the disease (Harris, E D. Pathogenesis of rheumatoid arthritis. In: UpToDate, Schur, P H (Ed), UpToDate, Wellesley, Mass., 2008). As the new vessels develop, inflammatory cells accumulate in the synovium and synovial fluid and release pro-inflammatory cytokines such as IL-1B that propagate the inflammatory response and lead to tissue destruction. Among the inflammatory cells present in the synovium of RA patients are eosinophils, neutrophils, T-lymphocytes, and importantly monocytes and macrophages which secrete TNF-α and IL-1β, two cytokines that play a central role in the pathophysiology of RA (Goldblatt et al. Clinical and Experimental Immunology, 140:195-204, 2005). The current line of therapy includes the use of disease modifying antirheumatic drugs (DMARDs), glucocorticoids, anticytokine therapies such as IL-1B receptor antagonist, methotrexate, and others. However, despite the availability of these therapies approximately 30 percent of patients with RA fail to respond adequately to therapy (Helfgott, S M. Evaluation and medical management of end-stage rheumatoid arthritis. In: UpToDate, Schur, P H (Ed), UpToDate, Wellesley, Mass., 2008).

### Inflammatory Bowel Disease

Inflammatory Bowel Diseases (IBD) consists of Crohn's disease and ulcerative colitis which produce inflammation in the digestive tract. Crohn's disease is characterized by a transmural, granulomatous inflammation occurring anywhere in the alimentary canal, but is usually centered in the terminal ileum and ascending colon. The inflammation associated with Crohn's disease is characterized by “skip lesions” consisting of areas of inflammation alternating with areas of normal mucosa. The affected area of bowel in Crohn's is marked by erythema, edema, and increased friability. When inflammation is present for a long time (chronic), it sometimes can cause scarring (fibrosis). Clinical signs/symptoms of Crohn's disease can include but are not limited to: cachexia, and poor growth, abdominal pain, draining fisulae, rectal prolapse and dehydration. Ulcerative colitis, in contrast, is marked by a superficial inflammation causing epithelial cell destruction (ulceration) that is centered in the rectum and colon. Unlike Crohn's disease, ulcerative colitis only affects one section of the inner lining of the colon starting from the rectum. Ulcerative colitis can be classified into several areas of the digestive tract, but contain common symptoms of bloody or loose stools, inflammation, abdominal pains, dehydration, and weight loss.

The idiopathic inflammatory bowel diseases (Crohn's disease and ulcerative colitis) are due to inappropriate and/or excessive responses to antigens present in the normal bacterial micro flora. Bacterial products, such as lipopolysaccharide (LPS), stimulate the recruitment of inflammatory cells and the release of cytokines (Segain, J P Gastroenterology; 124: 1180-1187, 2003). This recruitment of inflammatory cells and release of cytokines contribute to the inflammation of the digestive tract. Defect in epithelial barrier function is a common occurrence in those with inflammatory bowel disease, and may contribute PMN infiltration into the intestinal mucosa. Disease activity in IBD is linked to the transepithelial influx of neutrophils, ultimately leading to the formation of crypt abscesses in the intestinal lumen (Kucharzik, T Am. J, of Path.; 159: 2001-2009, 2001). Symptoms associated with inflammatory bowel disease affect the daily lives and quality of people. The prevalence of IBS in North America estimated from population-based studies is approximately 10 to 15 percent and in Europe is found to be 11.5 percent (Chun, et. al. Clinical manifestations and diagnosis of irritable bowel syndrome. In: UpToDate, Lamont J T (Ed), UpToDate, Wellesley, Mass., 2008). In many studies, it has been suggested that relatives with a first-degree relation to a person affected with IBD will have a 4 to 20 times higher chance of acquiring the disease over the general population (Podolsky, P D, N Engl J Med; 347 (6): 417-29, 2002). Out of those people with symptoms of IBD, only 15 percent seek medical attention. Regardless of the small amount of people seeking medical attention, IBD results in 25 to 50 percent of all gastroenterologist referrals. IBD affects the financial situation for many people inflicted with the condition. IBD is the second highest cause of work absentees after the common cold and has been interrelated to higher health care costs with an annual total of $30 billion (Chun, et. al. Clinical manifestations and diagnosis of irritable bowel syndrome. In: UpToDate, Lamont J T (Ed), UpToDate, Wellesley, Mass., 2008).

Present treatments for inflammatory bowel disease include anti-inflammatory drugs, immune system suppressors, or surgery. Anti-inflammatory drugs consist of azulfidines, colazals, salicylate, and corticosteroids. While these drugs prove somewhat beneficial, there are numerous side effects such as vomiting, increased diarrhea, high blood pressure and diabetes, bone fractures, mild kidney inflammation, and stunted growth and can also be used only short-term. After long term use of corticosteroids, side effects can include thinning of the bone and skin, infections, diabetes, muscle wasting, rounding of faces, psychiatric disturbances, and destruction of hip joints. Immune system repressors can be used for longer amounts of time, but because the drugs suppress the immune system, fatal infection and contraction of other immune diseases is more prevalent. Surgery is recommended for those who do not respond to oral medications. Surgery often makes daily tasks difficult due to protocolectomy and the requirement of wearing a small bag to collect stools.

### Neuropathic Pain

Neuropathic pain is chronic pain caused by dysfunction of the peripheral or central nervous system without continuing tissue damage. This includes pain due to neuropathic and idiopathic pain syndromes, and pain associated with neuropathic-related disorders such as cancer, HIV, multiple sclerosis, shingles, spine surgery, diabetic neuropathy, causalgia, brachial plexus avulsion, occipital neuralgia, fibromyalgia, gout, and other forms of neuralgia. Neuropathic pain often involves neural hypersensitivity and can persist without any overt external stimulus. (Goodman & Gilman's “the Pharmacologic Basis of Therapeutics”, 1996, p. 529, McGraw Hill).

The therapeutic objective of most pain therapy is to alleviate the symptoms of pain regardless of the cause. Current pain control therapies include the use of opioids, NSAIDs or ion channel blockers, all of which have safety profiles that are a cause for concern. Individuals with neuropathic disorders and the resulting debilitating neuropathic pain have a decreased quality of life, but agents commonly used to treat other types of pain are usually ineffective (Beniczky S et al. J Neural Transm, 112 (6):735-49, 2005).

There exists a need for methods of preventing or treating diseases of disorders caused by enhanced P2X₇ activity. Particularly, there exists a need for safe and effective methods for preventing or treating inflammation, inflammatory conditions, neurodegenerative diseases, and pain.

### Alzheimer's Disease

Alzheimer's Disease (AD) is a dementing disorder characterized by progressive impairments in memory and cognition. It typically occurs in later life, and is associated with a multiplicity of structural, chemical and functional abnormalities involving brain regions concerned with cognition and memory. Alzheimer's disease is characterized by neurofibrillary tangles (NFTs) and by extracellular amyloid aggregates. The disease typically begins in patients between 60 to 80 years old and progresses to dementia within 5 years and death in approximately 10 years. Current first-line therapies for Alzheimer's disease are cholinesterase inhibitors which enhance the half-life of the acetylcholine in cholinergic synapses involved in learning and memory. NMDA antagonists have recently been approved and are thought to work by decreasing NMDA associated excitotocity. (Alexander, M et al. Treatment of Dementia. In: UpToDate, Rose, B D (Ed), UpToDate, Wellesley, Mass., 2008). Despite the availability of these agents, AD continues to be a debilitating disease and new treatment options are needed. It is therefore clear that there remains today a long standing need for a treatment of AD before the disease has manifested far enough to produce psychological changes, thereby allowing earlier and more effective therapeutic intervention. Furthermore, these treatments do not address the underlying cause of the disease. Alzheimer's has been linked to the toxic 42-amino acid long, amyloid-β (Aβ) peptides, as the primary cause of amyloid aggregates. IL1 is thought to have a role in Alzheimer's disease on the basis of its overexpression in the brains of afflicted patients; its ability to induce excessive expression of the b-amyloid precursor protein; and its ability to activate astrocytes to produce a number of important proteins related to Alzheimer's disease, including S100b, IL6, a1-antichymotrypsin, and apolipoprotein E.31 Notably, the number of activated microglia that overexpress IL1 has been correlated with the number of b-amyloid plaques (Hallegua et al. Ann Rheum Dis 2002, 61:960).

### Multiple Sclerosis

Demyelinating diseases are those in which the main pathogenic process causes the destruction of the myelin sheath, which is necessary for the integrity of central nervous system cells. Among demyelinating diseases, multiple sclerosis (MS) is the most frequent disease due to alteration of the myelin in the central nervous system and, with the exception of trauma, it is the most frequent cause of neurological impairment in young adults. It affects 1.5 million people worldwide, and its symptoms generally occur in young adults, therefore its consequences at a personal and socioeconomic level are very severe. (Noseworthy et al., New Engl. J. Med., 343:938-952, 2000) Susceptibility to MS is due to unknown genetic and environmental factors.

There is a consensus among MS researchers according to which the disease has two stages, an initial inflammatory phase of an autoimmune nature, followed by a secondary progressive neurodegenerative phase. In the first phase, activated T cells cross the hematoencephalic barrier, and once inside the central nervous system, they release proinflammatory cytokines triggering an immunological cascade ending in the destruction of the myelin and death of the oligodendrocytes. Knowledge of the autoimmune process with certain detail has served to develop agents of an immuno-modulating nature, the therapeutic efficacy of which is very modest. Until now, different targets for intervention during the inflammatory phase of MS (Zamvil et al., Neuron 38:685-688, 2003) have been disclosed. Among them are those which are focused on reducing inflammation of the nervous system initiated by the activation of the myelin-specific T cells, promoting autoimmunity particularly against components of the myelin, entering the central nervous tissue and releasing in it pro-inflammatory cytokines such as interferon-γ and tumor-α necrosis factor. The immuno-modulator interferon-1β, approved for the treatment of remitting-recurrent MS, also prevents cellular interactions leading to the penetration of activated T cells through the vascular endothelium. Other treatments in clinical trial phase are focused on neutralizing the activity of proinflammatory cytokines and/or to enhance anti-inflammatory ones. It has been demonstrated that peripheral blood mononuclear cells (PBMCs) from MS patients produce increased amount of NO and in turn, PBMCs with larger amount of NO were shown to produce or secrete larger amount if IL-1B. It has also been described that patients at the moment of diagnosis of the disease and prior to any immunosuppressive therapy exhibit reduced levels of IL10 expression (Musette et al. Res Immunol. 1996, 147 (7):435). IL10 has been shown to have immunosuppressive effect on astrocyte response to IL-1beta. These findings support targeting IL1B via P2X₇ antagonism as a potentially effective way to reduce levels of a proinflammatory cytokine with a likely role in the pathogenesis of the disease.

At this time, no medication has been generated which delays or stops the progression of the neurodegenerative phase of the disease which takes a course with progressive neurological degeneration, and which is characterized by the occurrence of severe demyelinating lesions in the white substance with massive oligodendrocyte loss, atrophy and severe axonal damage.

MS treatments developed to date have focused on slowing the progression of the disease or moderating its symptoms. One treatment includes medicating the patients with either interferon beta-1b or an alternative, glatiramer, both of which will block the immune system's attack on myelin. The following are medications that treat the symptoms of MS: corticosteroids that will reduce inflammation of the nerve tissue, muscle relaxants, and amantadine and modafinil that will reduce fatigue (The Mayo Clinic-MS Treatments [online] 2008 [cited June 18] www.mayoclinic.org/multiple-sclerosis/treatment.html).

### Dry Eye

There are many ocular conditions where it is therapeutically desirable to correct improper tear fluid production. Dry eye is the general term for disease abnormalities that impact the pre-corneal tear film leading to a loss of mucous-containing goblet cells of the conjunctiva and eventually desquamation of the corneal epithelium that leads to destabilization of the cornea-tear interface (Gilbard J et al. CLAO Journal 22 (2), 141-45 (1996)). There are several main structures responsible for maintaining the properties of the tear film such as the glands and ducts surrounding the eye and the ocular surface. These structures maintain the tear film via regulation of water and electrolyte transport and via mucin release by goblet cells. Among the ocular conditions where disruption of one of these structures can cause or lead to “dry eye disease” are: keratoconjunctivitis sicca (KCS), age-related dry eye, Stevens-Johnson syndrome, Sjogren's syndrome, ocular cicatrical pemphigoid, blepharitis, corneal injury, infection, Riley-Day syndrome, congenital alacrima, nutritional disorders or deficiencies, pharmacologic side effects, eye stress and glandular and tissue destruction, environmental exposure to smog, smoke, excessively dry air, airborne particulates, autoimmune and other immunodeficient disorders, and comatose patients rendered unable to blink. This is not to be considered an exhaustive list but is used to describe some of the diseases that can lead to dry eye disease.

Treatment for dry eye disease is effective regulation of the tear film. This can be accomplished by enhancing natural production or improving flow from the glands surrounding the eye or applying artificial tears to the ocular surface. The glands can be blocked due to inflammation of the surrounding tissue or the duct and gland itself. Blockage due to inflammation can be seen by increases in pro-inflammatory cytokines, redness and puffiness on and surrounding the ocular surface. Reduction of this inflammation can help return tear production to normal function and improve corneal health. (Wilson S et al. American Academy of Opthalmology 114 (1), 76-79 (2007)). Increased IL-1B levels have been demonstrated in experimental dry eye models (Zhu et al. Mol. Vis. 2009, 15:250) and have been linked to increased expression of proinflammatory cytokines (Narayanan et al Cornea 2008, 27 (7):811) and aqueous tear deficiency, and destruction of lacrimal gland acinar epithelial cells (Zoukhri et al. Exp. Eye Res. 2007, 84 (5):894).

Currently, the pharmaceutical treatment of dry eye disease is mostly limited to administration of artificial tears (saline solution) to temporarily rehydrate the eyes and to reduction of inflammation ((Riento K et al. Nat Rev Mol Cell Biol, 4:446-456, 2003)). However, artificial tears, the most widely used group of products, often have contraindications and incompatibility with soft contact lenses (Lemp M et al. Cornea 9 (1), S48-550 (1990)).

### Blepharitis

Blepharitis, also known as Lid Margin Disease (LMD), is a non-contagious inflammation of the eyelids that manifests itself through scaling and flaking around the eyelashes, excess sebum production and oily scaly discharge, mucopurulent discharge, and matted, hard crusts around the lashes. Accumulation of crust, discharge or debris on the eyelashes and lid margins creates an ideal environment for overgrowth of the staphylococcal bacteria naturally found on the skin of the eyelids and increases the chance of infection, allergic reaction and tear break down. Blepharitis disturbs the production of the critical, outer lipid layer of the tear film which causes the entire tear to evaporate, resulting in dry eye. A reduced tear quantity doesn't properly dilute bacteria and irritants, nor wash inflammatory products away from the lashes and lid margin, so they accumulate and lead to further inflammation worsening the cycle of disease, with blepharitis, meibomian gland dysfunction and dry eye perpetuating each other.

Routine examination of the eyelids of blepharitis patients shows redness caused by capillary congestion (erythema) as well as crusting of the lashes and lid margins. More detailed inspection using a high magnification slit lamp microscope reveals additional features, including loss of lashes (madarosis), whitening of the lashes (poliosis), scarring and misdirection of lashes (trichiasis), crusting of the lashes and meibomian orifices, eyelid margin ulcers, plugging of the meibomian orifices, and lid irregularity (tylosis).

Blepharitis is a common eye disorder throughout the Unites States and the world. There is an apparently high incidence in the general population based on the frequency of diagnoses in ophthalmologists' offices. It affects people of all ages; however blepharitis caused by seborrhea is seen more often in older patients around the age of fifty. Chronic blepharitis has been associated with occupations in which the hands are dirty for much of the day, since poor hygiene is a risk factor. Acute blepharitis results most commonly from an allergic reaction to a drug or chemical substance. Likewise, exposure to irritants such as chemical fumes, smoke, and environmental pollutants can exacerbate the condition of chronic blepharitis. The use of certain drugs can also cause blepharitis. It has been documented that some patients on cancer chemotherapeutic agents such as 5-fluorouracil develop ocular surface and lacrimal complications, including blepharitis, conjunctivitis, keratitis, and eyelid dermatitis (Eiseman A S et al. Ophthal Plast Reconstr Surg, 19:3:216-224, 2003).

Designing an effective treatment plan for blepharitis can be challenging. Treatment includes good hygiene and relies heavily on the patient as a partner in achieving disease management. Since lid scrubs and hot compresses are required multiple times daily, long-term compliance to produce positive results can be an issue. If left untreated, blepharitis can lead to a more serious condition called ulcerative blepharitis accompanied by eyelid scarring, scarring of the cornea, and eventually loss of visual function.

It is well known that during acute and chronic inflammation various putative mediators of inflammation are released by the inflamed tissues and by leukocytes. The concentrations of these mediators and leukocytes are indicative of the level or degree of inflammation. Likewise, a reduction in concentration of these mediators and leukocytes is an indication of the effectiveness of a drug in treating inflammation. Anti-inflammatory steroidal preparations (e.g., corticosteroids) are currently the drug of choice in the treatment of ocular inflammatory conditions. The use of a topical ophthalmic steroid can be helpful in reducing acute inflammation, however extended use is complicated by severe and numerous side effects. It would be highly desirable to develop new nonsteroidal drugs which have a high therapeutic effectiveness but which do not exhibit steroid-like side effects.

### SUMMARY OF THE INVENTION

The present invention is directed to a method for preventing or treating pulmonary, autoimmune and ophthalmic diseases associated with inflammation in a mammal. The present invention is also directed to a method for inhibiting the release of interleukin-1β in a mammal, thus preventing or treating inflammatory conditions mediated by interleukin-1β and its proinflammatory effects. The present invention is further directed to a method for preventing or treating neurodegenerative diseases, or pain in a mammal.

The present invention is particularly useful in treating diseases associated with pulmonary inflammation such as asthma and chronic obstructive pulmonary disease, as well as inflammatory bowel disease, rheumatoid arthritis, and neuropathic pain.

The methods comprise the steps of: identifying a mammal in need thereof, and administering to the mammal an effective amount of a compound of Formula I, VIa, VIb, VIIa, VIIb, or a pharmaceutically acceptable salt, tautomer, hydrate, or solvate thereof.

The present invention is further directed to novel mononucleotide Compounds 1-56.

### BRIEF DESCRIPTION OF THE DRAWINGS

FIG. 1 shows the reduction of neutrophil accumulation in bronchoalveolar lavage Fluid.

FIG. 2A shows the reduction of IL-1β in bronchoalveolar lavage fluid by systemic (i.p.) dosing. FIG. 2B shows the reduction of IL-1β in bronchoalveolar lavage fluid by pulmonary (i.t.) dosing.

### DETAILED DESCRIPTION OF THE INVENTION

### Definitions

When present, unless otherwise specified, the following terms are generally defined as, but are not limited to, the following:

“Alkyl” groups are from 1 to 12 carbon atoms inclusively, either straight chained or branched, are more preferably from 1 to 8 carbon atoms inclusively, and most preferably 1 to 6 carbon atoms inclusively.

“Alkylene chains” are from 2 to 20 carbon atoms inclusively, have two points of attachment to the molecule to which they belong, are either straight chained or branched, can contain one or more double and/or triple bonds, are more preferably from 4 to 18 atoms inclusively, and are most preferably from 6 to 14 atoms inclusively.

“Alkenyl” groups are from 1 to 12 carbon atoms inclusively, either straight or branched containing at least one double bond but can contain more than one double bond.

“Alkynyl” groups are from 1 to 12 carbon atoms inclusively, either straight or branched containing at least one triple bond but can contain more than one triple bond, and additionally can contain one or more double bonded moieties.

“Alkoxy” refers to the group alkyl-O— wherein the alkyl group is as defined above including optionally substituted alkyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl-groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl-groups preferably having from 1 to 6 carbon atoms in the alkenyl moiety and from 6 to 10 carbon atoms inclusively in the aryl moiety.

“Arylalkynyl” refers to aryl-alkynyl-groups preferably having from 1 to 6 carbon atoms inclusively in the alkynyl moiety and from 6 to 10 carbon atoms inclusively in the aryl moiety.

“Aryloxy” refers to the group aryl-O— wherein the aryl group is as defined above including optionally substituted aryl groups as also defined above.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl-groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylmethyl, cyclohexylethyl, adamantlymethyl, and the like.

“Halo” substituents are taken from fluorine, chlorine, bromine, and iodine.

“Heteroaryl” refers to a monovalent aromatic carbocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl-groups preferably having from 1 to 6 carbon atoms inclusively in the alkyl moiety and from 6 to 10 carbon atoms inclusively in the heteroaryl moiety. Such arylalkyl groups are exemplified by pyridylmethyl and the like.

“Heteroarylalkenyl” refers to heteroaryl-alkenyl-groups preferably having from 1 to 6 carbon atoms inclusively in the alkenyl moiety and from 6 to 10 carbon atoms inclusively in the heteroaryl moiety.

“Heteroarylalkynyl” refers to heteroaryl-alkynyl-groups preferably having from 1 to 6 carbon atoms inclusively in the alkynyl moiety and from 6 to 10 carbon atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Positions occupied by hydrogen in the foregoing groups can be further substituted with substituents exemplified by, but not limited to, hydroxy, oxo, nitro, methoxy, ethoxy, alkoxy, substituted alkoxy, fluoro, chloro, bromo, iodo, methyl, ethyl, propyl, butyl, alkyl, substituted alkyl, thio, thioalkyl, acyl, carboxyl, alkoxycarbonyl, carboxamido, substituted carboxamido, alkylsulfonyl, alkylsulfinyl, alkylsulfonylamino, sulfonamido, substituted sulfonamide, cyano, amino, substituted amino, acylamino, trifluoromethyl, trifluoromethoxy, phenyl, aryl, substituted aryl, pyridyl, imidazolyl, heteroaryl, substituted heteroaryl, cyclopropyl, cyclobutyl, cyclopentyl, cyclohexyl, cycloalkyl, substituted cycloalkyl, pyrrolidinyl, piperidinyl, morpholino, and heterocycle; and preferred heteroatoms are oxygen, nitrogen, and sulfur. It is understood that where open valences exist on these substituents they can be further substituted with alkyl, cycloalkyl, aryl, heteroaryl, and/or heterocycle groups, and where multiple such open valences exist, these groups can be joined to form a ring, either by direct formation of a bond or by formation of bonds to a new heteroatom, preferably oxygen, nitrogen, or sulfur. It is further understood that the above subtitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₄.

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of Formula I is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definition of compounds in Formula I encompasses all possible hydrates and solvates, in any proportion, which possess the stated activity.

The term “edema” refers to an abnormal accumulation of extra-vascular fluid. Of particular relevance here is “pulmonary edema” which refers specifically to fluid accumulation within the lung interstitium or the lumen of the lung. Pulmonary edema is associated with a variety of systemic or lung diseases including respiratory syncytial virus infection (RSV), human metapneumovirus, pneumonia, influenza, ventilator induced lung injury (VILI), acute respiratory distress syndrome (ARDS), acute lung injury (ALI), and chronic obstructive pulmonary disease (COPD) such as chronic bronchitis and emphysema.

“Inflammation” generally refers to a localized reaction of tissue, characterized by the influx of immune cells, which occurs in reaction to injury or infection. Specifically, “pulmonary inflammation” is characterized by migration of inflammatory cells into the interstitium and the lumen of the lung, release of pro-inflammatory cytokines and chemokines, lung tissue remodeling and lung tissue apoptosis or necrosis. Pulmonary inflammation accompanies a variety of systemic or lung diseases including those noted in the aforementioned pulmonary edema definition.

“An effective amount” is the amount effective to treat a disease by ameliorating the pathological condition or reducing the symptoms of the disease. “An effective amount” is the amount effective to improve at least one of the parameters relevant to measurement of the disease.

The present invention is directed to methods of inhibiting P2X₇ receptor activation via antagonizing the effect of ATP at the P2X₇ receptor by P2X₇ antagonist compounds of Formula I. The P2X₇ antagonist compounds can be competitive antagonists, inverse agonists, negative allosteric modulators, or indirect modulators of receptor function. P2X₇ antagonism lowers the release of pro-inflammatory cytokines and other pro-inflammatory mediators including IL-1β, TNF-A and MMP9. P2X₇ antagonism also inhibits the leukocyte migration into tissue of interest, elastin degradation, collagen deposition, lung tissue fibrosis and remodeling, emphysema, mucus cell metaplasia, mucus secretion and cartilaginous tissue destruction.

The present invention is directed to a method for inhibiting the release of interleukin-1β in vitro and in vivo.

In one embodiment, the present invention is directed to methods of preventing, treating or ameliorating diseases or conditions of the lung associated with excessive inflammation and remodeling. Particularly, this invention is directed to methods of treating pulmonary diseases with inflammatory or remodeling pathophysiologies such as asthma, chronic obstructive pulmonary disease, respiratory tract illness caused by respiratory syncytial virus, idiopathic pulmonary fibrosis, acute respiratory distress syndrome and ventilator induced lung injury, cystic fibrosis, bronchiectasis, alpha-1-antitrypsin deficiency, rhinitis, rhinosinusitis, primary ciliary dyskinesia, pneumonia, bronchiolitis caused by agents other than respiratory syncytial virus, OB/BOOP due to lung transplantation of HSCT, non IPF-IIPs and OB/BOOP.

In another embodiment, this invention is directed to methods for preventing, treating or ameliorating of non-pulmonary diseases with inflammatory and remodeling component such as rheumatoid arthritis, inflammatory bowel disease, osteoarthritis and inflammatory bowel syndrome.

In yet another embodiment of this invention, this invention is directed to methods for preventing, treating or ameliorating of pain (neuropathic pain, chronic inflammatory pain, and visceral pain) and neurodegeneration due to inflammation as encountered in multiple sclerosis or Alzheimer's disease.

In yet further embodiment, this invention is directed to methods for preventing, treating or ameliorating ophthalmic diseases associated with inflammation such as dry eye or blepharitis.

The methods comprise the steps of: identifying a mammal in need thereof, and administering to the mammal an effective amount of a mononucleoside compound of Formula I.

An effective amount of a Formula I compound is administered to a patient in need of such treatment. The patient either already has the symptoms of at least one above-mentioned disease, or is identified as being at risk of at least one above-mentioned disease. The compound is administered at a frequency that achieves desired efficacy. What constitutes desired efficacy is determined by a physician or other health-care professional. Whether or not sufficient efficacy has been reached is determined by indicia of efficacy for the specific disease. After an initial dose, additional doses are optionally administered if judged to be necessary by a health-care professional.

### Mononucleoside Compounds

The present invention provides mononucleoside compounds of Formula I, and/or tautomers thereof, and/or pharmaceutically-acceptable salts, and/or solvates, and/or hydrates thereof; which are useful for preventing and/or treating diseases associated with abnormalities in the activity of P2X₇ receptor.

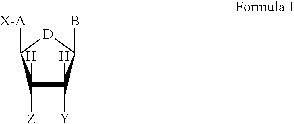

wherein:
X is a covalently bound substituent selected from the group —R₁, —OR₁, —COOR₁, —SR₁, —SO₃H, —S(O₂)NR₁R₂, —NR₁(CO)R₂, —NR₁(SO₂)R₂, —NR₁(SO₂)NR₁R₂, —(CO)NR₁R₂, —NH(CO)NR₁R₂, —NR₁(NH)NR₁R₂, —NR₁(CO)OR₂, —O(CO)NR₁R₂, —NR₁R₂, —OP(O)(OH)₂, —CH₂—P(O)(OH)₂, where R₁ and R₂ are independently H, alkyl, alkenyl, alkynyl, alkoxy, aryl, arylalkyl, arylalkenyl, arylalkynyl, aryloxy, cycloalkyl, cycloalkenyl, cycloalkylalkyl, heteroaryl, heteroarylalkyl, heteroarylalkenyl, heteroarylalkynyl, or heterocycle, as previously defined; provided any resultant structure is reasonable from a chemical or stability point of view; and any moiety X containing a group capable of existing in a charged form (for example, COOH and NR₁R₂) may be taken to mean either the neutral or charged species;
D=O or CH₂, giving a ribose or carbocycle ring, respectively;
A is CH₂, CH═CH, or absent; and
for all moieties defined by A-X, the atom that is directly attached to the 4′ carbon of a ribose ring is not N, O, or S, and provided that the resultant structure is reasonable from a chemical or stability point of view;
B is a purine or a pyrimidine residue according to general Formulae III and IV, respectively, which is linked to the 1′ position of the furanose or carbocycle via the 9- or 1-position of the base, respectively;
Y and Z are taken together to form a moiety according to Formula II, leading to an acetal or a ketal at the 2′/3′ position of the ribose ring, with ketal being preferred

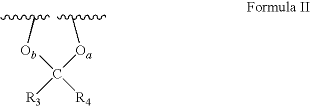

wherein:
Oₐ and O_{b} are respectively the 2′ and 3′ oxygens of the furanose or carbocycle; and Oₐ and O_{b} are linked by a common carbon atom (C) to form an acetal or ketal;
R₃ and R₄ are independently H, alkyl, alkenyl, alkynyl, cycloalkyl, cycloalkenyl, aryl, arylalkyl, arylalkenyl, arylalkynyl, with or without substitution; or can be joined together to form a homocyclic or heterocyclic ring composed of 3 to 8 atoms, preferably 3 to 6 atoms;
B is a purine or a pyrimidine residue according to general Formulae III and IV, respectively, which is linked to the 1′ position of the furanose or carbocycle via the 9- or 1-position of the base, respectively;

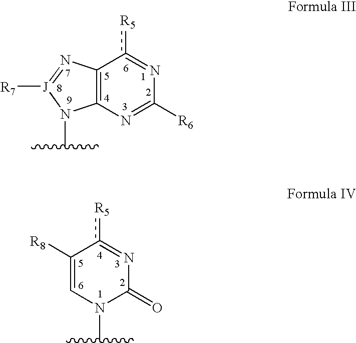

wherein:
R₅ is hydroxy, oxo, amino, mercapto, alkylthio, arylthio, alkyloxy, aryloxy, cycloalkyloxy, cycloalkylalkyloxy, alkylamino, cycloalkylamino, cycloalkylalkylamino, aralkylamino, arylamino, diaralkylamino, or diarylamino; or
R₅ is dialkylamino, where the alkyl groups are optionally linked to form a ring; and alkyl moieties falling under the definition of R₅ optionally contain saturated or unsaturated bonds, or substitutents, provided that the resultant moiety is chemically reasonable, and aryl moieties falling under the definition of R₅ optionally contain substitutents on the ring or heteroatoms within the ring, provided that the resultant moiety is chemically reasonable; or
R₅ is acylamino;
R₇ is hydrogen, alkyl, bromo, azido, alkylamino, arylamino or aralkylamino, alkoxy, aryloxy or aralkyloxy, alkylthio, arythio or aralkylthio;
J is carbon or nitrogen, with the provision that when nitrogen, R₇ is not present;
R₆ is hydrogen, halo, amino, monosubstituted amino, disubstituted amino, alkylthio, arylthio, or aralkylthio; and
R₈ is hydrogen, halo, alkyl, alkenyl, or alkynyl.

Compounds according to Formulae III and IV, where R₅ is acylamino for the most part fall within the scope of Formula V:

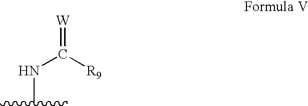

wherein NH is the amino residue at the C-6 position in a purine or the amino residue at the C-4 position in a pyrimidine;
W is oxygen or sulfur; and R₉ is amino or mono- or disubstituted amino such that the moiety according to Formula V is a urea or thiourea; or R₉ is alkoxy, aralkyloxy, aryloxy, substituted aralkyloxy, or substituted aryloxy, such that the moiety according to Formula V is a carbamate or thiocarbamate; or
R₉ is alkyl, alkenyl, alkynyl, cycloalkyl, cycloalkenyl, aryl, arylalkyl, arylalkenyl, arylalkynyl, with or without substitution, with or without heteroatoms, such that the moiety according to Formula V is an amide.

In a preferred embodiment, neither R₃ or R₄ is H. Preferably, R₃ and R₄ are joined to form a cycloalkyl ring, such as a cyclohexyl and cyclopentyl ring; or R₃ and R₄ are joined to form an aracycloalkyl ring, such as an indanone ring.

When B is a purine, it is preferably adenosine.

When B is a pyrimidine, it is preferably cytidine.

A preferred formula of the present invention is Formula VIa (B is a purine):

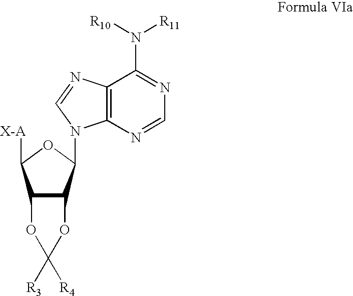

wherein:

A, X, R₃, and R₄ are as previously defined;

R₁₀ and R₁₁ are independently H, alkyl, alkenyl, alkynyl, cycloalkyl, cycloalkenyl, aryl, arylalkyl, arylalkenyl, arylalkynyl, cycloalkylalkyl, heteroaryl, heteroarylalkyl, heteroarylalkenyl, heteroarylalkynyl, or heterocycle with or without substitution; or

when neither R₁₀ and R₁₁ is H, they are optionally joined together by a saturated or unsaturated bond, forming a ring of from 3 to 8 atoms, with or without substituents, unsaturation, or heteroatoms (beyond the requisite nitrogen atom of the 6-amino moiety).

Another preferred formula of the present invention is Formula VIb (B is a pyrimidine):

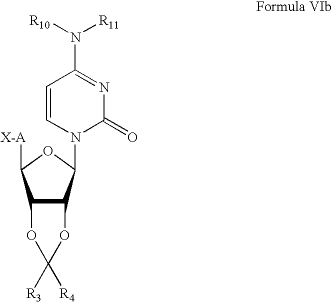

wherein A, X, R₃, R₄, R₁₀ and R₁₁ are as previously defined for Formula VIa;

Another preferred formula is Formula VIIa:

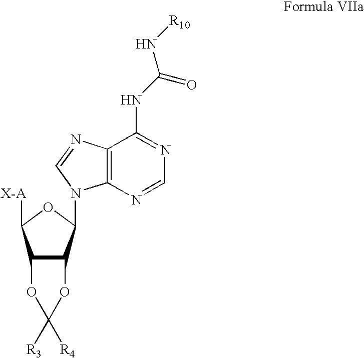

wherein A, X, R₃, and R₄ are as previously defined for Formula VIa;

R₁₀ is alkyl, alkenyl, alkynyl, cycloalkyl, cycloalkenyl, aryl, arylalkyl, arylalkeryl, arylalkynyl, cycloalkylalkyl, heteroaryl, heteroarylalkyl, heteroarylalkenyl, heteroarylalkynyl, or heterocycle with or without substitution.

Another preferred formula is Formula VIIb:

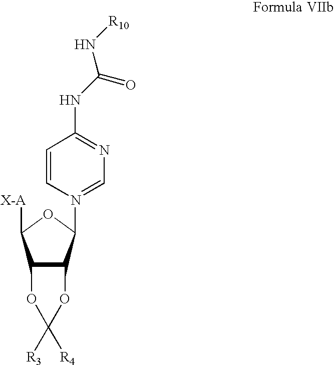

wherein A, X, R₃, R₄ and R₁₀ are as previously defined for Formula VIIa.

For Formulae VIa, VIb, VIIa, and VIIb, when A is CH₂, the preferred X is OH, NR₁(CO)R₂, —O(CO)NR₁R₂, NH(CO)NR₁R₂, or —NR₁R₂, such that the combined moiety X-A is respectively an alcohol, an amide, a carbamate, a urea or an amine; or

when A is a carbonyl, the preferred X is OH, or —NR₁R₂, such that the combined moiety A-X is respectively a carboxylic acid or an amide.

For Formulae VIa, VIb, VIIa, and VIIb, R₃ and R₄ are preferably joined together to form a six membered cylcoalkyl ring (cyclohexyl ring) or a bicyclic aracycloalkyl ring (2-indanone ring).

For Formulae VIa and VIb, R₁₀ and R₁₁ are preferably H, cycloalkyl, cycloalkylalkyl, aryl, or arylalkyl, provided both are not H; or

when neither R₁₀ and R₁₁ are H, R₁₀ and R₁₁ are optionally joined together by a bond such that they form a ring of from 3 to 6 atoms;

For Formulae VIIa and VIIb, R₁₀ is preferably cycloalkyl, cycloalkylalkyl, aryl, or arylalkyl.

The following compounds are novel and useful for the present invention.

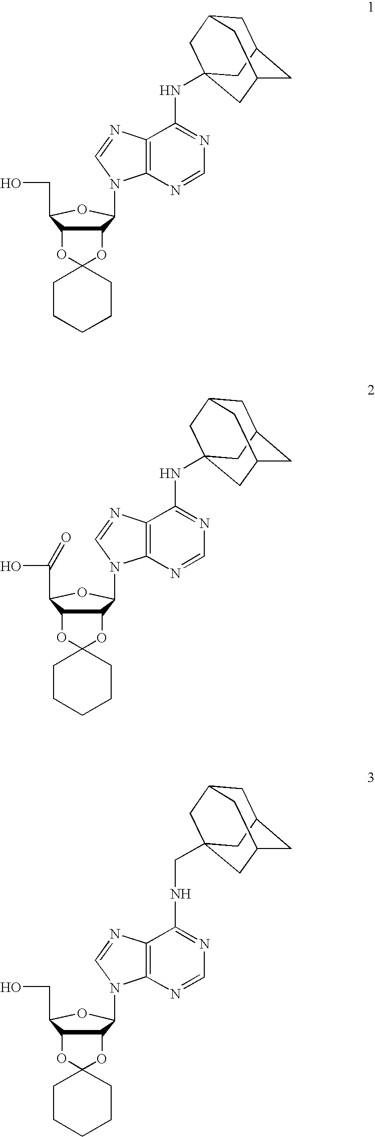

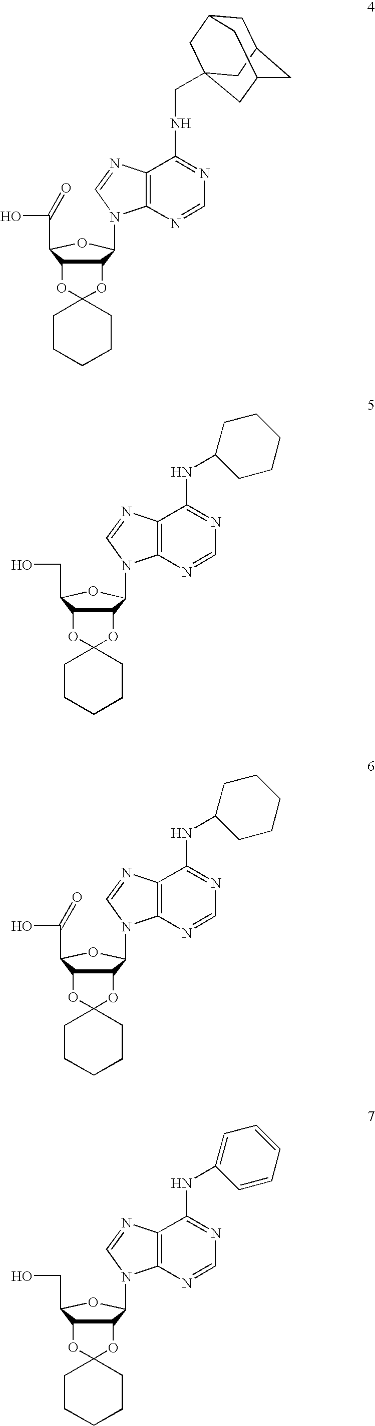

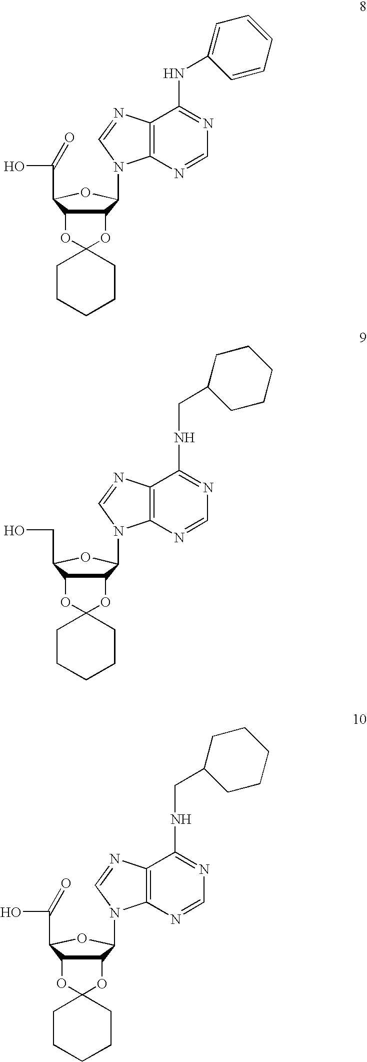

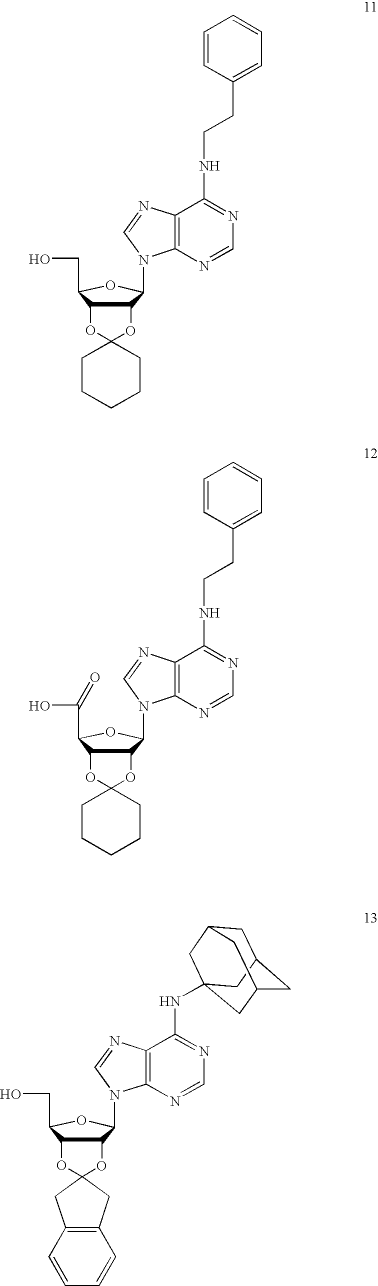

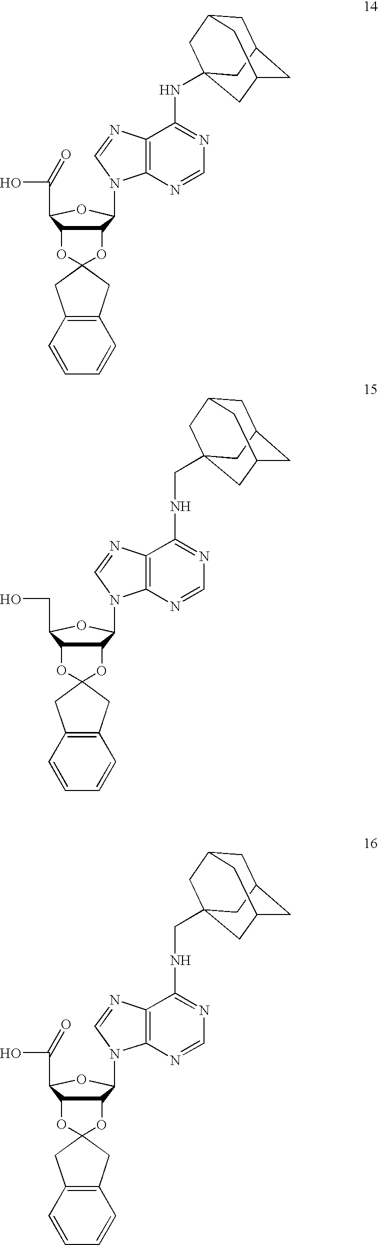

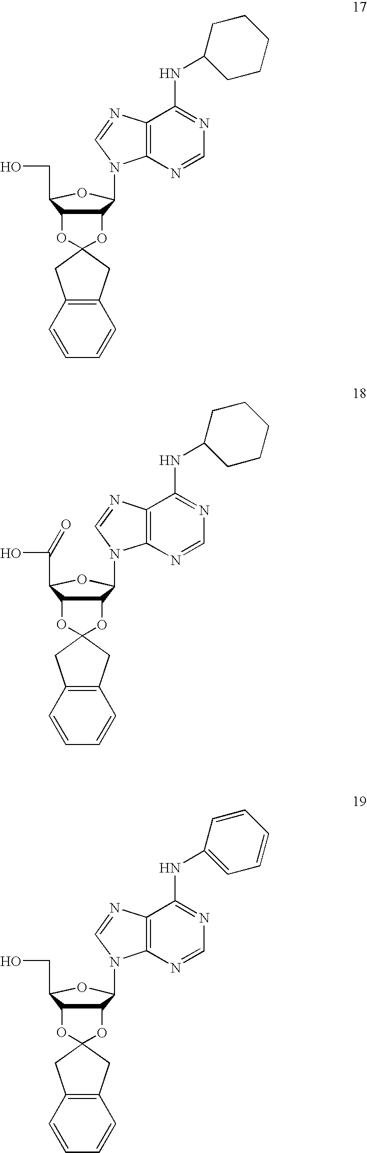

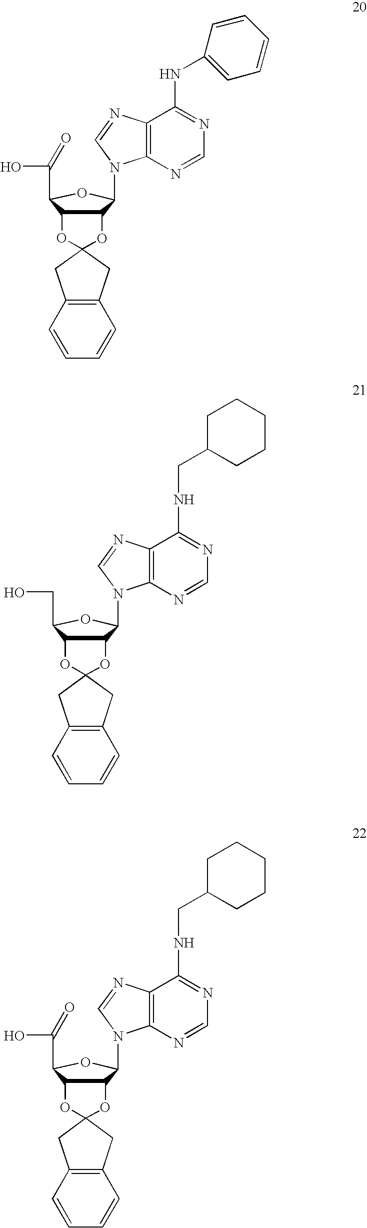

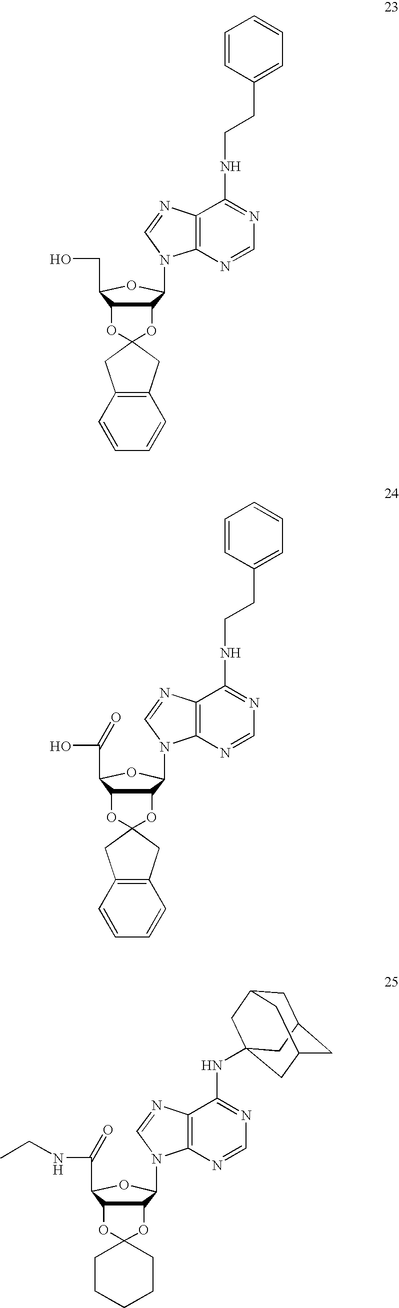

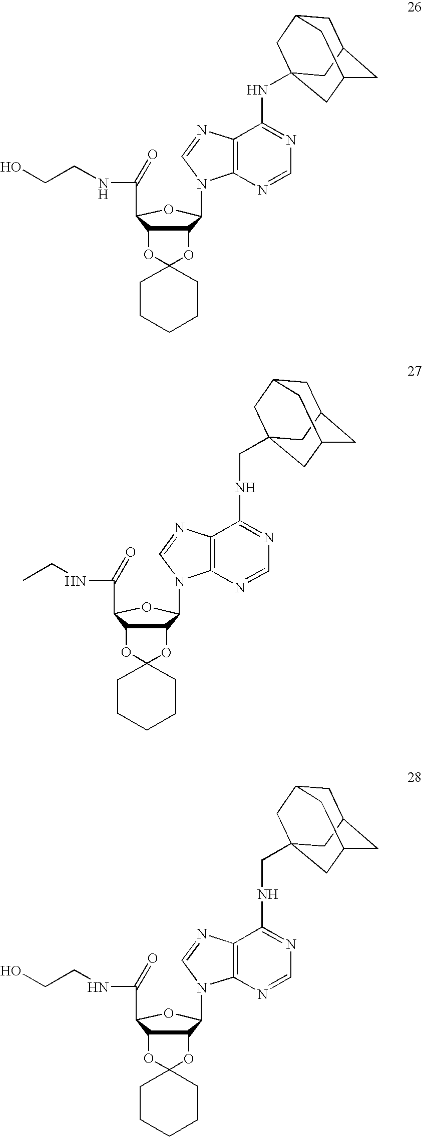

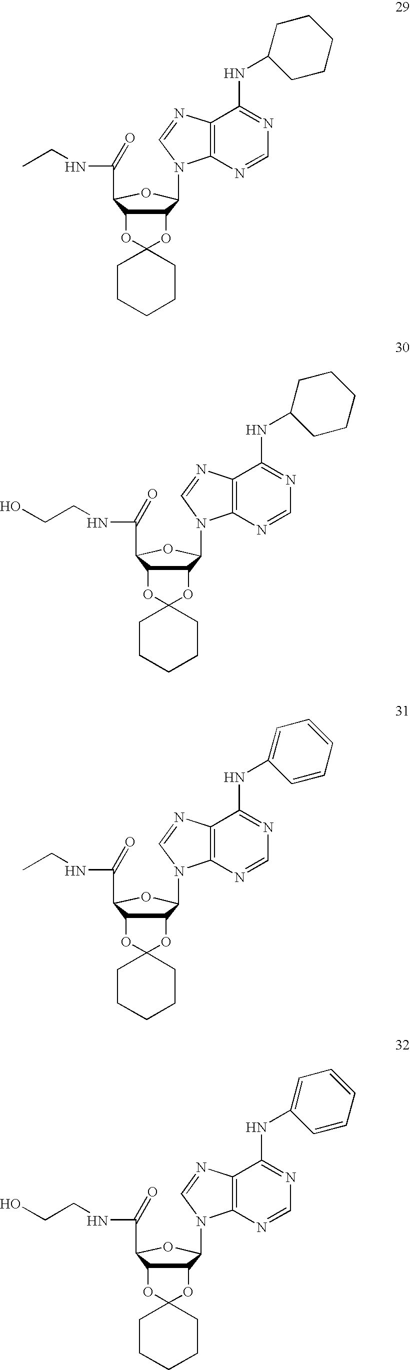

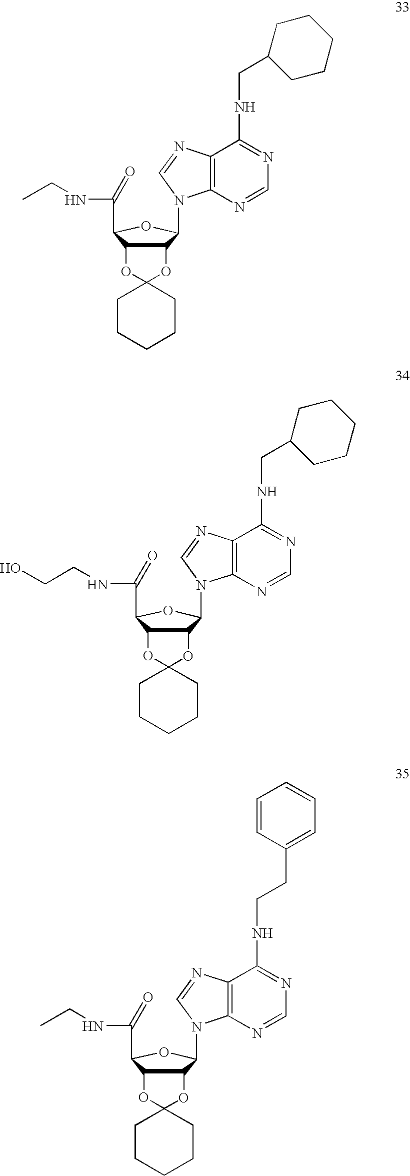

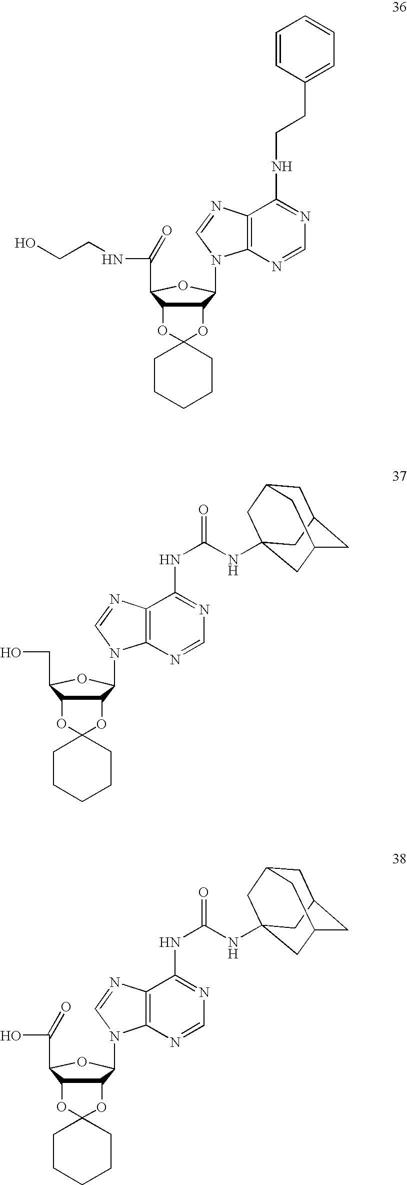

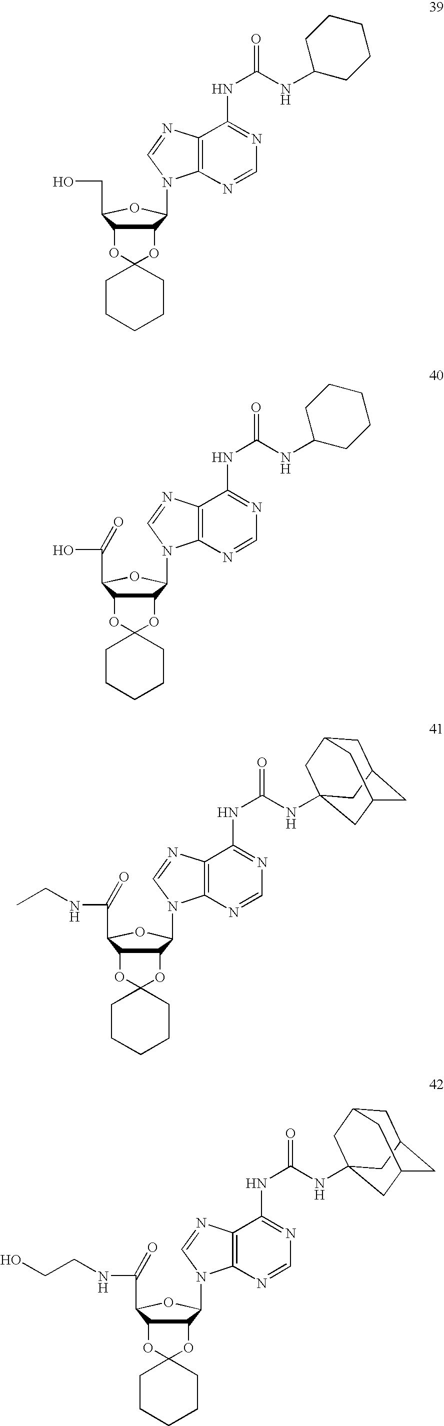

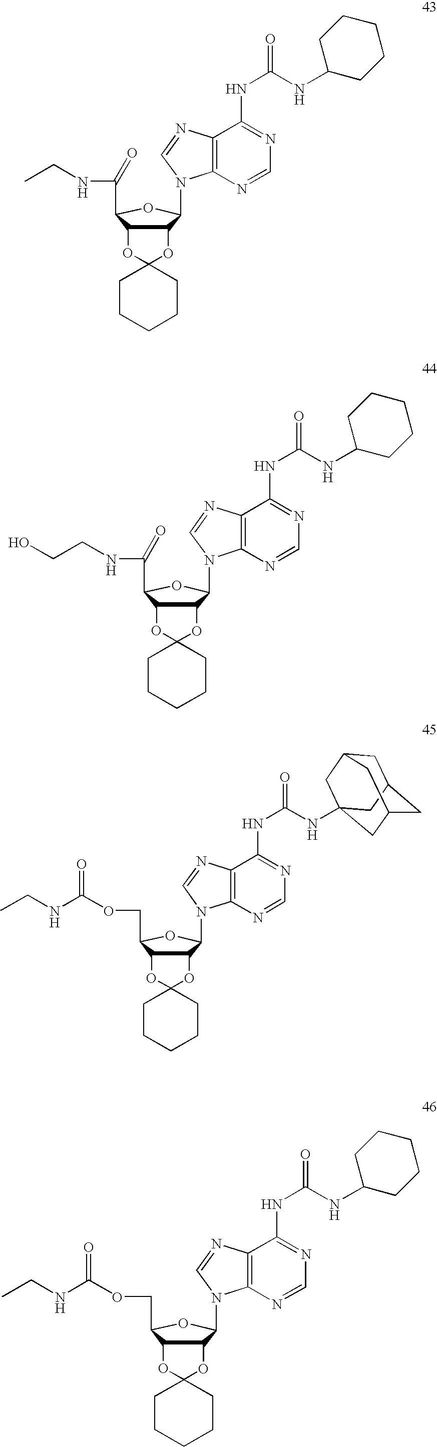

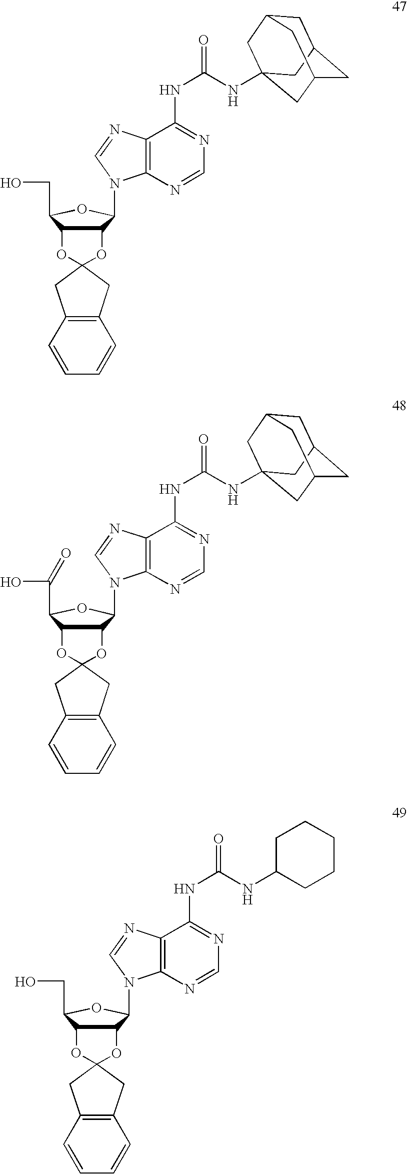

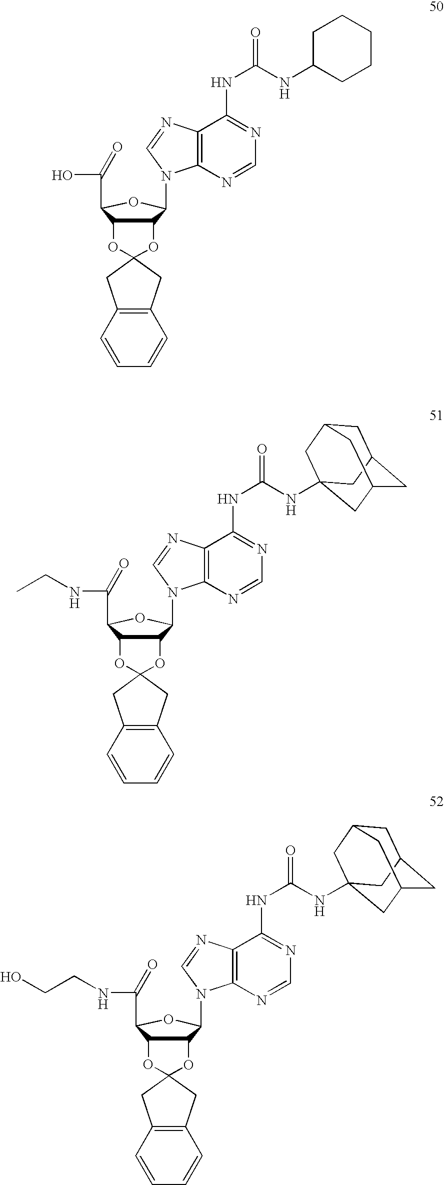

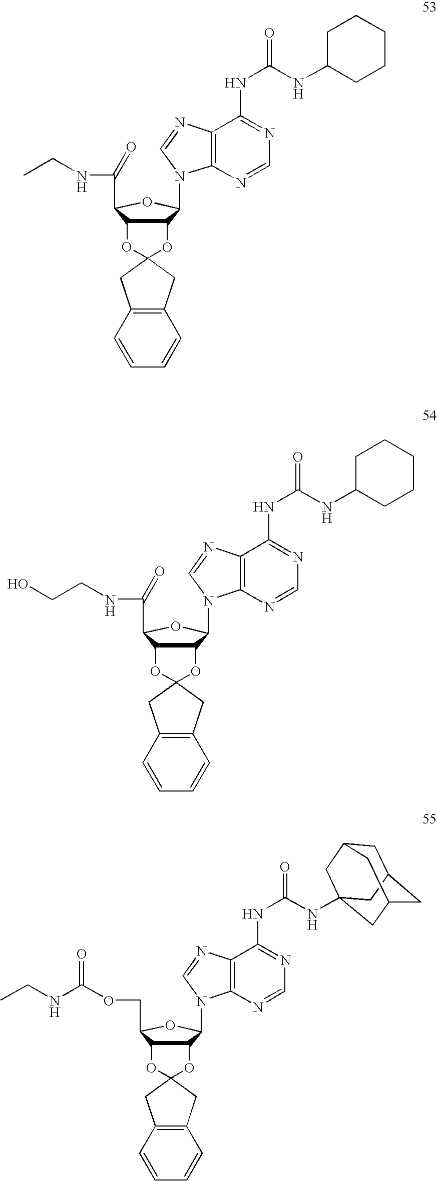

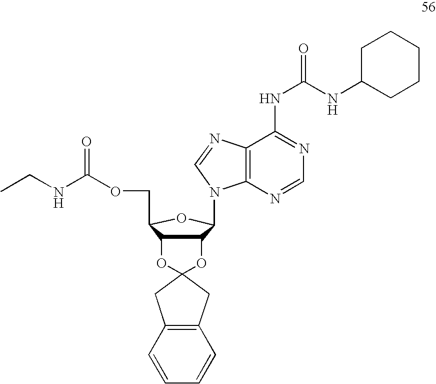

### Preparation of Compounds

The compounds of the present invention can be prepared according to the following general schemes 1 and 2, which are offered by way of illustration and not of limitation:

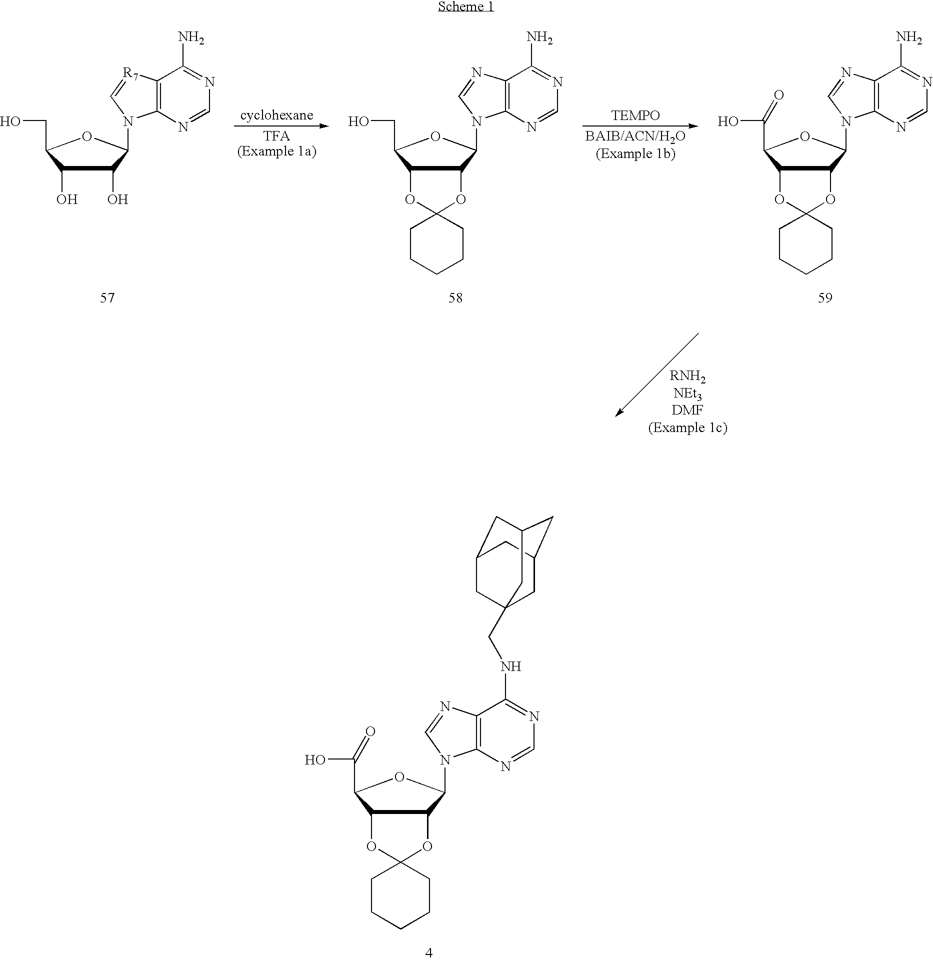

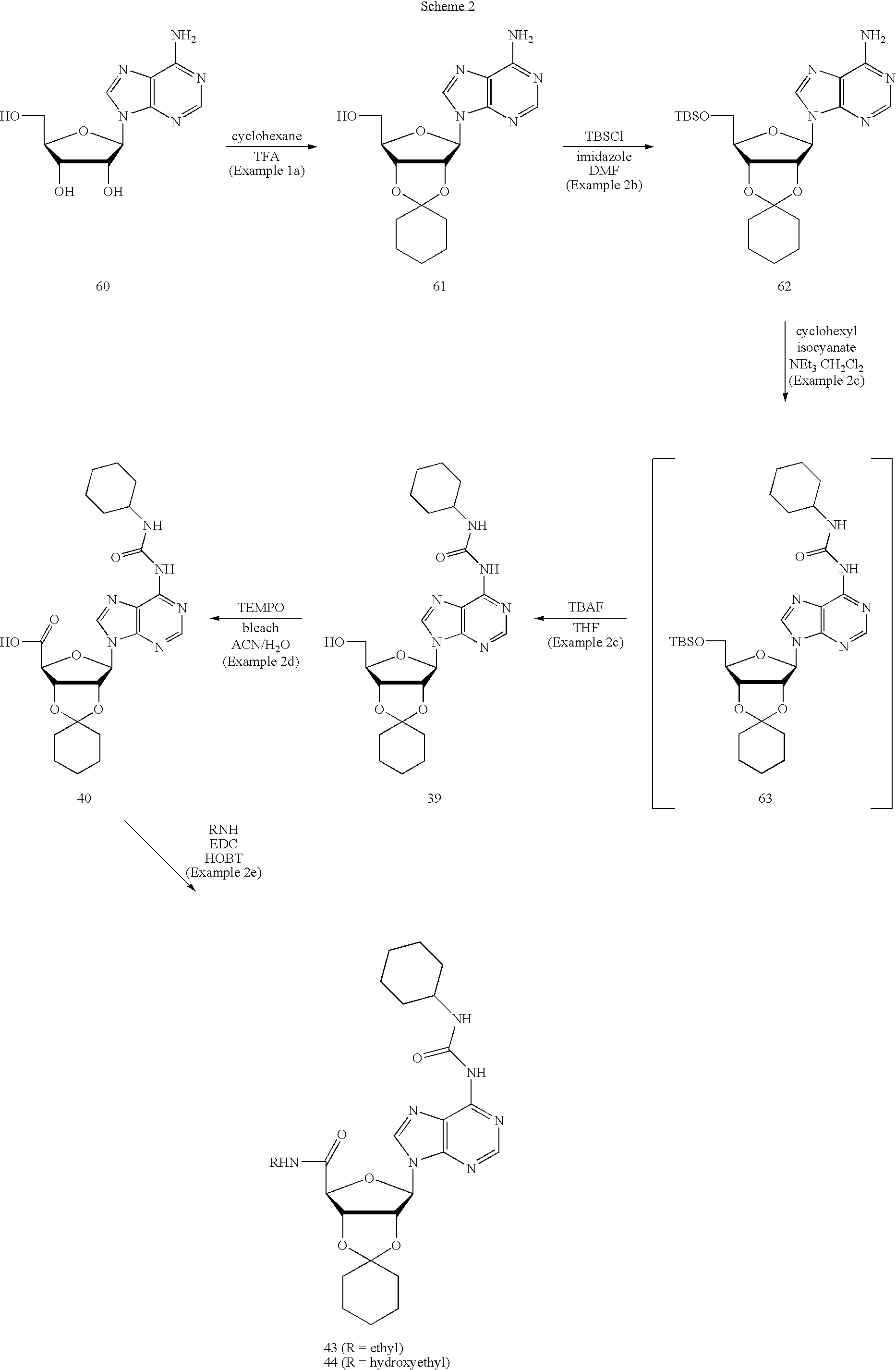

In the general Formula I, the substituents at Y and Z are taken together to form acetals or ketals, as described by Formula II. Acetals and ketals can be readily prepared by reaction of the neighboring 2′ and 3′ hydroxyl groups in an appropriate nucleoside or nucleotide with an aldehyde or ketone, respectively, or their chemical equivalents, in the presence of an acid catalyst. Typical acids include trichloroacetic, p-toluenesulfonic, and methanesulfonic employed in catalytic amounts, in conjunction with inert solvents. Alternately, weaker organic acids such as formic can be used as both the catalyst and solvent for the reaction.

When the nucleoside or nucleotide to be derivatized is a purine that contains a 6-amino functionality or is a pyrimidine that contains a 4-amino functionality, it can be converted to the respective urea or thiourea, as described by general formula V. This can be accomplished by treatment with isocyanates or isothiocyanates, respectively, as depicted for the synthesis of compound 63 in Scheme 2.

Alternately, the 6 amino position of a purine or the 4 position of a pyrimidine can be converted to a carbamate by treatment with a suitable haloformate, or a suitable chemical equivalent.

Alternately, the 6 amino position of a purine or the 4 position of a pyrimidine can be converted to an amide by reaction with a suitably activated carboxylic acid or a carboxylic acid in conjunction with an appropriate coupling reagent.

When the nucleoside or nucleotide to be derivatized is a purine that contains a 6-chloro functionality or is a pyrimidine that contains a 4-chloro functionality, it can be converted to the corresponding substituted amino analogs. This can be accomplished by treatment of the chloro analog with an appropriate amine, as depicted for the synthesis of compound 4 in Scheme 1.

Those skilled in the art will recognize various synthetic methodologies, which can be employed to prepare non-toxic pharmaceutically acceptable salts when ionizable groups are present and prodrugs of the compounds of the present invention.

### Pharmaceutical Formulations and Methods of Administration

The present invention additionally provides a pharmaceutical formulation comprising compounds of Formula I and a pharmaceutically acceptable carrier. Pharmaceutically acceptable carriers can be selected by those skilled in the art using conventional criteria. Pharmaceutically acceptable carriers include, but are not limited to, saline solution, aqueous electrolyte solutions, isotonicy modifiers, water polyethers such as polyethylene glycol, polyvinyls such as polyvinyl alcohol and povidone, cellulose derivatives such as methylcellulose and hydroxypropyl methylcellulose, polymers of acrylic acid such as carboxypolymethylene gel, polysaccharides such as dextrans, and glycosaminoglycans such as sodium hyaluronate and salts such as sodium chloride and potassium chloride.

The pharmaceutical formulation of the present invention provides an aqueous solution comprising water, suitable ionic or non-ionic tonicity modifiers, suitable buffering agents, and a compound of Formula I. In one embodiment, the compound is at 0.005 to 3% w/v, and the aqueous solution has a tonicity of 200-400 mOsm/kG and a pH of 4-9.

The pharmaceutical formulation can be sterilized by filtering the formulation through a sterilizing grade filter, preferably of a 0.22-micron nominal pore size. The pharmaceutical formulation can also be sterilized by terminal sterilization using one or more sterilization techniques including but not limited to a thermal process, such as an autoclaving process, or a radiation sterilization process, or using pulsed light to produce a sterile formulation. In one embodiment, the pharmaceutical formulation is a concentrated solution of the active ingredient; the formulation can be serially diluted using appropriate acceptable sterile diluents prior to systemic administration.

In one embodiment, the tonicity modifier is ionic such as NaCl, for example, in the amount of 0.5-0.9% w/v, preferably 0.6-0.9% w/v.

In another embodiment, the tonicity modifier is non-ionic, such as mannitol, dextrose, in the amount of at least 2%, or at least 2.5%, or at least 3%, and no more than 7.5%; for example, in the range of 3-5%, preferably 3.5-5%, and more preferably 4.2-5% w/v.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or acetyl alcohol. Sweetening agents such as those set forth above, and flavoring agents can be added to provide palatable oral preparations. These compositions can be preserved by the addition of an anti-oxidant such as ascorbic acid.

Pharmaceutical compositions of the invention can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions can also contain sweetening and flavoring agents.

The compounds of the present invention can be administered by oral, topical, parenteral, inhalation, or other systemic administration.

This inhalation method involves an aerosol suspension of respirable particles comprising the active compound, which the subject inhales. The respirable particles can be liquid or solid, with a particle size sufficiently small to pass through the mouth and larynx upon inhalation. In general, particles having a size of about 1 to 10 microns, preferably 1-5 microns, are considered respirable.

The surface concentrations of active compounds delivered via inhalation can vary according to compounds; but are generally 1×10⁻¹⁰-1×10⁻⁴ moles/liter, and preferably 1×10⁻⁸-1×10⁻⁵ moles/liter.

For systemic administration such as injection and infusion, the pharmaceutical formulation is prepared in a sterile medium. The active ingredient, depending on the vehicle and concentration used, can either be suspended or dissolved in the vehicle. Adjuvants such as local anesthetics, preservatives and buffering agents can also be dissolved in the vehicle. The sterile injectable preparation can be a sterile injectable solution or suspension in a non-toxic acceptable diluent or solvent. Among the acceptable vehicles and solvents that can be employed are sterile water, saline solution, or Ringer's solution.

Another method of systemic administration of the active compound involves oral administration, in which pharmaceutical compositions containing active compounds are in the form of tablets, lozenges, aqueous or oily suspensions, viscous gels, chewable gums, dispersible powders or granules, emulsion, hard or soft capsules, or syrups or elixirs.

For oral use, an aqueous suspension is prepared by addition of water to dispersible powders and granules with a dispersing or wetting agent, suspending agent one or more preservatives, and other excipients. Suspending agents include, for example, sodium carboxymethylcellulose, methylcellulose and sodium alginate. Dispersing or wetting agents include naturally-occurring phosphatides, condensation products of an allylene oxide with fatty acids, condensation products of ethylene oxide with long chain aliphatic alcohols, condensation products of ethylene oxide with partial esters from fatty acids and a hexitol, and condensation products of ethylene oxide with partial esters derived from fatty acids and hexitol anydrides. Preservatives include, for example, ethyl, and n-propyl p-hydroxybenzoate. Other excipients include sweetening agents (e.g., sucrose, saccharin), flavoring agents and coloring agents. Those skilled in the art will recognize the many specific excipients and wetting agents encompassed by the general description above.

For oral application, tablets are prepared by mixing the active compound with nontoxic pharmaceutically acceptable excipients suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Formulation for oral use can also be presented as chewable gums by embedding the active ingredient in gums so that the active ingredient is slowly released upon chewing.

Additional means of systemic administration of the active compound to the lungs of the subject would involve a suppository form of the active compound, such that a therapeutically effective amount of the compound reaches the target sites via systemic absorption and circulation.

For rectal administration, the compositions in the form of suppositories can be prepared by mixing the active ingredient with a suitable non-irritating excipient which is solid at ordinary temperatures but liquid at the rectal temperature and will therefore melt in the rectum to release the compound. Such excipients include cocoa butter and polyethylene glycols.

The active compounds can also be systemically administered to the lungs of the subject through absorption by the skin using transdermal patches or pads. The active compounds are absorbed into the bloodstream through the skin. Plasma concentration of the active compounds can be controlled by using patches containing different concentrations of active compounds.

One systemic method involves an aerosol suspension of respirable particles comprising the active compound, which the subject inhales. The active compound would be absorbed into the bloodstream via the lungs. The respirable particles can be liquid or solid, with a particle size sufficiently small to pass through the mouth and larynx upon inhalation; in general, particles ranging from about 1 to 10 microns, but more preferably 1-5 microns, in size are considered respirable.

Another method of systemically administering the active compounds to the lungs of the subject involves administering a liquid/liquid suspension in the form of eye drops or eye wash or nasal drops of a liquid formulation, or a nasal spray of respirable particles that the subject inhales. Liquid pharmaceutical compositions of the active compound for producing a nasal spray or nasal or eye drops can be prepared by combining the active compound with a suitable vehicle, such as sterile pyrogen free water or sterile saline by techniques known to those skilled in the art.

For systemic administration, plasma concentrations of active compounds delivered can vary according to compounds; but are generally 1×10⁻¹⁰-1×10⁻⁴ moles/liter, and preferably 1×10⁻⁸-1×10⁻⁵ moles/liter.

Dosage levels about 0.01-140 mg per kg of body weight per day are useful in the treatment or preventions of conditions involving an inflammatory response (about 0.5 mg to about 7 g per patient per day). Preferred dosage levels are about 0.05-25, or 0.1-10 mg/kg body weight per day. The amount of active ingredient that can be combined with the carrier materials to produce a single dosage form will vary depending upon the host treated and the particular mode of administration. Dosage unit forms will generally contain between from about 1 mg to about 500 mg of an active ingredient.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more can be administered to achieve adequate steady state levels. The maximum total dose in general does not exceed about 2 g/day for a 40 to 80 kg human patient.

Frequency of dosage can also vary depending on the compound used and the particular disease treated. However, for treatment of most disorders, a dosage regimen of 4 times daily, three times daily, or less is preferred, with a dosage regimen of once daily or 2 times daily being particularly preferred.

It is understood that the specific dose level for any particular patient depends upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

Preferred compounds of the invention have favorable pharmacological properties. Such properties include, but are not limited to bioavailability (e.g., oral bioavailibilty, preferably high enough to permit oral administration of doses of less than 2 grams, preferably of less than or equal to one gram), low toxicity, low serum protein binding and desirable in vitro and in vivo half-life. Distribution in the body to sites of complement activity is also desirable, e.g., compounds used to treat CNS disorders will preferably penetrate the blood brain barrier, while low brain levels of compounds used to treat peripheral disorders are typically preferred.

Assays can be used to predict these desirable pharmacological properties. Assays used to predict bioavailability include transport across human intestinal cell monolayers, including Caco-2 cell monolayers. Toxicity to cultured hepatocycles can be used to predict compound toxicity. Penetration of the blood brain barrier of a compound in humans can be predicted from the brain levels of the compound in laboratory animals given the compound intravenously.

### Methods of Administration for Treating Pulmonary Inflammation

The present invention is particularly useful for treating pulmonary inflammation. Any method of delivering the compound to the lumen of the lung, including local administration and systemic administration, is suitable for the present invention. Systemic administration is introducing a medicament into the circulation. Examples of systemic administration include oral ingestion, or intravenous or subcutaneous or intraperitoneal or intrathecal or intramuscular administration.

A preferred embodiment of the invention is localized administration. Local administration includes inhalation, topical application or targeted drug delivery. Methods of inhalation include liquid instillation, instillation as a pressurized fluid preparation via metered dose inhaler or equivalent, or inhalation of an aerosolized solution via nebulizer (preferred), inhalation of dry powder (more preferred), and directing soluble or dried material into the air stream during mechanical ventilation (also more preferred).

An example of targeted drug delivery is enclosure of the compound within a liposome, where the liposome is coated with a specific antibody whose antigen is expressed in the targeted lung tissue.

It can be advantageous to construe a controlled delivery system of the compounds since such an inhaled product targets the site of action, presents the compound of interest in small regimented quantities and reduces/minimizes any unwanted side effects.

Another example of a delivery system includes microparticulates compositions of the compound. In such a case, the compound is formulated as a microparticulate wherein the carrier is loaded with the compound; such a preparation is then filtered through a fine porous membrane or suitable filtering medium or is exposed to solvent interchanges to produce nanoparticles. Such preparations can be freeze dried or held in suspension in an aqueous or physiologically compatible medium. The preparation so obtained can be inhaled by suitable means.

Another example of a suitable preparation includes a reconstitutable preparation. In this case, the compound is formulated in a preparation to contain the necessary adjuvant to make it physiologically compatible. Such a preparation can be reconstituted by addition of water for injection or suitable physiological fluids, admixed by simple agitation and inhaled using appropriate techniques described above.

The compounds described above can also be prepared into dry powder or equivalent inhalation powders using the well known art of super critical fluid technology. In such a case, the compound is admixed with appropriate excipients and milled into a homogenous mass using suitable solvents or adjuvants. Following this, this mass is subjected to mixing using super critical fluid technology and suitable particle size distribution achieved. The particles in the formulation need to be of a desired particle size range such that the particles can be directly inhaled into the lungs using a suitable inhalation technique or introduced into the lungs via a mechanical ventilator. Alternatively, a formulation can be designed such that the particles are large enough in size thereby offering sufficient surface area to dissolve completely in a suitable fluid when admixed together or to dissolve sufficiently enough prior to nebulization into the lungs.

To prevent particle size growth and minimize crystal growth of the compound, one embodiment is to include the use of spray-dried particles that have better aerodynamic properties than micronized material. This can be further extended to coat the surface of the hydrophilic molecule with one or more layers of hydrophobic material.

### Clinical Applications

The compounds of the present invention modulate P2X₇ receptor function and are capable of antagonizing the effects of ATP at the P2X₇ receptor, thus they are useful in the prevention or treatment of inflammation and inflammatory conditions, neurodegenerative diseases, and pain.

Inflammation and the inflammatory conditions associated with said inflammation suitable to be treated by the present invention include pulmonary diseases such as asthma, chronic obstructive pulmonary disease, respiratory tract illness caused by respiratory syncytial virus, idiopathic pulmonary fibrosis, acute respiratory distress syndrome and ventilator induced lung injury, cystic fibrosis, bronchiectasis, alpha-1-antitrypsin deficiency, rhinitis, rhinosinusitis, primary ciliary dyskinesia, pneumonia, bronchiolitis caused by agents other than respiratory syncytial virus, OB/BOOP due to lung transplantation of HSCT, non IPF-IIPs and OB/BOOP; ophthalmic diseases such as glaucoma, retinitis, retinopathies, uveitis, acute injury to the eye tissue (e.g. conjunctivitis), dry eye, blepharitis; rheumatoid arthritis, Crohn's disease, ulcerative colitis, irritable bowel syndrome, and inflammatory bowel disease.

Neurodegenerative diseases suitable to be treated by the present invention include dementia, particularly degenerative dementia (including senile dementia, dementia with Lewy bodies, Alzheimer's disease, multiple sclerosis, Pick's disease, Huntingdon's chorea, Parkinson's disease and Creutzfeldt-Jakob disease, Amyotrophic Lateral Sclerosis (ALS) and motor neuron disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment associated with ageing, particularly Age Associated Memory Impairment.

Pain suitable to be treated by the present invention, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, cancer chemotherapy, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

### Pulmonary Inflammation

The present invention is directed to a method of treating asthma, COPD, RSV, IPF, ARDS and VILI, CF, bronchiectasis, AATD, rhinitis, rhinosinusitis, PCD, pneumonia, bronchiolitis caused by agents other than RSV, OB/BOOP due to lung transplantation of HSCT, non IPF-IIPs and OB/BOOP. The method comprises the steps of first identifying a subject suffering from asthma, COPD, RSV, IPF, ARDS and VILI, CF, bronchiectasis, AATD, rhinitis, rhinosinusitis, PCD, pneumonia, bronchiolitis caused by agents other than RSV, OB/BOOP due to lung transplantation of HSCT, non IPF-IIPs or OB/BOOP, then administering to the subject an effective amount of a compound of this invention to treat these pulmonary diseases.

A method for treating asthma, COPD, RSV, IPF, ARDS and VILI, CF, bronchiectasis, AATD, rhinitis, rhinosinusitis, PCD, pneumonia, bronchiolitis caused by agents other than RSV, OB/BOOP due to lung transplantation of HSCT, non IPF-IIPs or OB/BOOP is based on the properties of the compounds of this invention to reduce at least one of the following processes contributing to pathophysiologies that accompany this disorder: inflammation, remodeling, airway and/or lung tissue edema, airway hyperreactivity or bronchoconstriction.

Indicia of efficacy for treating asthma, COPD, RSV, IPF, ARDS and VILI, CF, bronchiectasis, AATD, rhinitis, rhinosinusitis, PCD, pneumonia, bronchiolitis caused by agents other than RSV, OB/BOOP due to lung transplantation of HSCT, non IPF-IIPs or OB/BOOP include demonstrable improvement in measurable signs, symptoms and other variables clinically relevant to these diseases. Such improvements include increased blood oxygen saturation, decreased hypoxia and hypercapnia, decrease need for supplemental oxygen, decreased frequency of coughing and/or wheezing, improved forced expiratory volume (FEV₁), forced vital capacity (FVC) or other physiologically relevant parameter of respiratory function, decreased need for mechanical ventilation, lower amount of inflammatory cells infiltrating the lung, lower levels of proinflammatory cytokines and chemokines, improved alveolar fluid clearance rate, decreased pulmonary edema as determined by any radiographic or other detection method such as amount of epithelial lining fluid, alveolar fluid clearance and/or radiographic visualization methods, increase in general quality of life, the levels of inflammatory cells in the lung or outside of the lung in other anatomical compartments or spaces including systemic circulation, the amount of pro-inflammatory molecules including cytokines and chemokines in the lung or outside of the lung in other anatomical compartments or spaces including systemic circulation, pathological remodeling of the airway, patient-reported or physician-observed signs such as ease of breathing, or severity of coughing and/or wheezing, decreased bilateral diffuse interstial infiltrates as determined by any radiographic or other detection method, improvement in histopathological changes of the pulmonary parenchyma, increase in general quality of life, improvement in gas exchange abnormalities including carbon monoxide diffusing capacity (DLCO), improvements in arthralgia, myalgia, hemoptysis, rash or pneumothorax.

### Rheumatoid Arthritis

The inventors have discovered that compounds of this invention inhibit cytokine secretion, edema, and subsequent leukocyte influx into tissues of interest. The inventors have therefore discovered that compounds of this invention are useful in treating the defects in inflammation and angiogenesis seen in Rheumatoid arthritis (RA). The present invention is directed to a method of treating RA. The method comprises the steps of first identifying a subject suffering from RA, then administering to the subject an effective amount of a compound of this invention to treat said disease.

A method for treating RA is based on the properties of a compound of this invention to reduce at least one of the following processes contributing to pathophysiologies that accompany this disorder cytokine secretion, edema, and subsequent leukocyte influx into tissues of interest.

Indicia of efficacy for treating rheumatoid arthritis include demonstrable improvements in measurable signs, symptoms and other variables relevant to RA. Such improvements include a decrease in swollen and tender joint counts, decrease in pain, improvements in patient and evaluator global assessments of disease activity, decrease in the duration of morning stiffness, decreased levels of fatigue, improvements in appetite and strength, resolution of fever, improved motion of wrist, elbow, neck, shoulder, hip and ankles joints, decreased swollen glands, decreased burning or itching sensation in eyes, inflammation, decreased numbness or tingling, decreased leg ulcers, decreased shortness of breath, improvement of the chronic inflammation of the tendon sheaths, decreased swollen lymph glands, decreased anemia, improved health status, and improved measures of function.

### Inflammatory Bowel Disease

The inventors have discovered that compounds of this invention inhibit cytokine secretion, edema, and subsequent leukocyte influx into tissues of interest. The inventors have therefore discovered that compounds of this invention are useful in treating the defects in inflammation, fibrosis, and edema seen in IBD. The present invention is directed to a method of treating IBD. The method comprises the steps of first identifying a subject suffering from IBD, then administering to the subject an effective amount of a compound of this invention to treat said disease.

A method for treating IBD is based on the properties of compounds of this invention to reduce at least one of the following processes contributing to pathophysiologies that accompany this disorder: cytokine secretion, edema, and subsequent leukocyte influx into tissues of interest.

Indicia of efficacy for treating inflammatory bowel disease include improvement in measurable signs, symptoms and other variables clinically relevant to inflammatory bowel disease. Improvements include: subsiding of an acute episode of disease, maintain non-inflammatory state, weight gain, attenuation of rectal bleeding and pain, decreased urgency or inability to move bowels, decrease in or subsiding of abdominal cramps or pain, alleviation of fatigue and dehydration, prevention of colon rupture and toxic megacolon, firmer stools, decrease in the occurrence of ulcers, reduction of fever, decrease in gastroesophageal reflux, lack of nausea, decrease in chest pain, decrease in abdominal bloating, decrease in gas production, increase in sexual desire, increase in urinary regularity, elimination of mucus from stools, decrease of diarrhea occurrence, decrease in signs of malnutrition, decrease in signs or occurrence of perianal disease, decrease in abdominal mass, decrease in fistulas and strictures, decrease in incidence of related cancers, decrease in inflammation, decrease in edema, decrease in epithelial cell destruction, decrease in fibrosis, decrease in mucous discharge, and decrease in tumor appearance.

### Neurodegenerative Diseases

The inventors have discovered that compounds of this invention inhibit P2X₇ receptor signaling and P2X₇ mediated cytokine and chemokine secretion and leukocyte infiltration. Furthermore, the inventors have discovered that compounds of Formula I are useful in treating the defects in neurodegeneration due to inflammation. The present invention is directed to a method of treating neurodegenerative diseases. The method comprises the steps of first identifying a subject suffering from a neurodegenerative disease, then administering to the subject an effective amount of a compound of Formula I to treat the neurodegenerative disease such as multiple sclerosis and Alzheimer's disease.

A method for treating neurodegenerative diseases is based on the properties of the compounds of this invention to reduce at least one of the following processes contributing to pathophysiologies that accompany this disorder: inflammation of the central or peripheral nervous system accompanied by excessive proinflammatory cytokines secretion and inflammatory cell accumulation or activation and demyelination or other remodeling of the central or peripheral nervous system.

Indicia of efficacy for treating of neurodegenerative disorders include demonstrable improvement in measurable signs, symptoms and other variables clinically relevant to the neurodegenerative disorders. Such improvements include decreased acute inflammation and demyelination in critical areas of the brain, optic nerves or spinal cord; decreased in number of lesions in the brain or spinal cord as detected by MRI; improvement in Poser criteria as defined in Poser, C M, Paty, D W, Scheinberg, L, et al, Ann Neurol 1983; 13:227; improvement in oligodendrocyte loss and astroglial scarring; decrease in undesirable sensory symptoms in limbs and face, visual loss, motor function loss, improvement in balance problems, improvement in vertigo, bladder problems, limb ataxia and pain; decrease in the frequency of attacks (an attack is defined as an episode of neurological disturbance for which causative lesions are likely to be inflammatory and demyelinating in nature); improvement in any of McDonald criteria or revised McDonald criteria as defined in Polman, C H, Reingold, S C, Edan, G, et al. Diagnostic criteria for multiple sclerosis: 2005 revisions to the “McDonald criteria”. Ann Neurol 2005; 58:840. Copyright© 2005 American Neurological Association; improvement, normalization or decrease in worsening of cerebro-spinal fluid analysis such as leukocyte cell count, IgG levels and oligoclonal bands; improvement, normalization or decrease in worsening of evoked potentials (EP) such as somatosensory EPs, visual EP and brainstem auditory EPs; improvement, normalization or decrease in worsening or cardinal motor features such as brady—and akinesia, rigidity, resting tremors and postural and gain instability; improvement, normalization or decrease in worsening of executive dysfunction, visuospatial impairments, memory loss and dementia.

### Multiple Sclerosis

The inventors have discovered that compounds of this invention inhibit P2X₇ receptor signaling and P2X₇ mediated cytokine and chemokine secretion and leukocyte infiltration. Furthermore, the inventors have discovered that compounds of Formula I are useful in treating the defects in neurodegeneration due to inflammation. The present invention is directed to a method of treating multiple sclerosis. The method comprises the steps of first identifying a subject suffering from multiple sclerosis, then administering to the subject an effective amount of a compound of this invention to treat said disease.

Indicia of efficacy for treating multiple sclerosis by the present invention include demonstrable improvements in visual symptoms including a decreased incidence of blurred vision and eye pain, recovering eyesight and color vision, not experiencing double vision or jerky eye movements, and experiencing coordination between two eyes and normal pupil responses. Improvements in motor symptoms consist of increased muscle strength, lack of muscle spasms, regaining muscle tone and posture, and decreased slurred speech and jerky muscle movements. Indications that the patient is improving in terms of sensation include a decreased frequency of numbness and tingling sensations, regaining sensation and awareness of body parts, and a lessening pain throughout the body and face. In terms of coordination and balance, the patient shows progress if they do not shake, regain coordination, can control limb movements, experience normal balance function in the inner ear, does not experience nausea or vomiting, can coordinate speech, and has ability to produce rhythmic movements. Improvements associated with the urogenital system comprise urinary continence and retention, erectile function, and normal ejaculation. Cognitive progress includes not suffering short-term and long-term memory problem and ability to comprehend speech. Other signs of improvement include a reduced incidence of acid reflux, epileptic seizures, and swallowing and respiratory difficulties.

### Alzheimer's Disease

The inventors have discovered that compounds of this invention inhibit P2X₇ receptor signaling and P2X₇ mediated cytokine and chemokine secretion and leukocyte infiltration. Furthermore, the inventors have discovered that compounds of Formula I are useful in treating the defects in neurodegeneration due to inflammation. The present invention is directed to a method of treating Alzheimer's disease. The method comprises the steps of first identifying a subject suffering from Alzheimer's disease, then administering to the subject an effective amount of a compound of this invention to treat said disease.

Indicia of efficacy for Alzheimer's disease include demonstrable improvement in measurable signs, symptoms and other variables clinically relevant to Alzheimer's. Such improvements include an increase in ability to make decisions, increase in focal motor skills, improved memory, a decrease in the number of unprovoked falls, fewer urinary symptoms not explained by urinary disease (frequency, urgency), fewer mood changes, decreased depression, decrease in number of hospital visits, decrease in length of hospital stay, increased feelings of well-being and improved quality of life, survival, improvement in the COGDRAS tests of attention and visuospatial orientation, in the Alzheimer's Disease Cooperative Study—Activities of Daily Living (ADCS-ADL) Inventory, in the Alzheimer's Disease Cooperative Study—Clinicians Global Impression of Change (ADCS-CGIC) or in the Alzheimer's Disease Assessment Scale Cognitive (ADAS-Cog).

### Neuropathic Pain

The inventors have discovered that compounds of this invention inhibit the neuro-remodeling associated with increased neuro-sensation associated with neuropathic pain. The present invention is directed to a method of treating neuropathic pain. The method comprises the steps of first identifying a subject suffering from neuropathic pain, then administering to the subject an effective amount of a compound of this invention to treat said disease.

Indicia of efficacy for neuropathic pain include demonstrable improvement in measurable signs, symptoms, and other variables clinically relevant to neuropathy. Such improvements include decrease in abnormal sensation, neural regeneration, neural functional recovery, reduced pain symptoms, improved sleeping patterns, decreased secondary myofacial pain, increased ambulatory activities, decreased abnormal skin sensation, decreased allodynia (painful response to non-painful stimulus), decreased hyperalgesia (increased painful response to painful stimulus), decreased ER/office visits, decreased length of hospital stay, decreased usage of unsafe pain therapies, decrease in missed work or school days, decreased depression, increased feelings of well-being and overall improved quality of life.

### Dry Eye

The inventors have discovered that compounds of this invention inhibit P2X₇ receptor signaling and P2X₇ mediated cytokine and chemokine secretion and leukocyte infiltration as seen in dry eye disease.

The present invention is directed to a method of treating dry eye. The method comprises the steps of first identifying a subject suffering from dry eye, then administering to the subject an effective amount of a compound of this invention to treat dry eye.

A method for treating dry eye is based on the properties of the compounds of this invention to reduce inflammation that accompany this disorder.

Indicia of efficacy for treating dry eye by the present method include demonstrable improvement in measurable signs, symptoms and other variables clinically relevant to dry eye. Such improvements include reducing the evaporation rate of normal or artificial tears, minimizing the loss of tears, maximizing the preservation of tears, increasing tear film stability, decreasing tear film osmolarity, increasing tear volume, increasing tear secretion, prolonging tear break-up time, decreasing immune-mediated inflammation, increasing gland function, decreasing irritation and itching, decreasing grittiness, decreasing foreign body sensation, increasing aqueous component of tears, decreasing photophobia, decreasing accumulation of mucus filaments, decreasing punctate conjunctival and corneal damage, inducing contraction of the bulbar conjunctival vessels, decreasing dullness of the conjunctiva and cornea, decreasing corneal punctate fluorescein staining, reducing symptoms of blurred vision, increasing secretion of natural anti-inflammatory factors and decreasing production of pro-inflammatory cytokines and proteolytic enzymes. Ophthalmic formulations containing compounds of this invention, that inhibit regulation of certain secreted pro-inflammatory factors and thus improve tear production and tear break up time by reducing immune-mediated inflammation, would clinically lead to decreased irritation and itching, decreased grittiness and foreign body sensation, decreased photophobia, a measurable decrease in corneal damage, contraction of the bulbar conjunctival vessels, decrease in corneal punctate fluorescein staining and reduced symptoms of blurred vision.

### Blepharitis

The inventors have discovered that compounds of this invention inhibit P2X₇ receptor signaling and P2X₇ mediated cytokine and chemokine secretion and leukocyte infiltration as seen in blepharitis.

The present invention is directed to a method of treating blepharitis. The method comprises the steps of first identifying a subject suffering from blepharitis, then administering to the subject an effective amount of a compound of this invention to treat blepharitis.

A method for treating blepharitis is based on the properties of the compounds of this invention to reduce at least one of the following processes contributing to pathophysiologies that accompany this disorder: inflammation and excessive proinflammatory cytokine secretion.

Indicia of efficacy for treating blepharitis by the present method include demonstrable improvement in measurable signs, symptoms and other variables clinically relevant to blepharitis. Such improvements include elimination of redness, swelling, burning, watering, and itching of the eyelids; decrease in flaking and debris accumulation on the eyelashes; decrease in a foreign body sensation; crusting and closure of eyelids upon waking; attenuation of abnormal growth or loss of lashes; decrease in pain sensation and sensitivity to light; a decrease in the incidence of associated complications such as styes, chalzions, dry eye, meibomitis, keratitis, and recurrent conjunctivitis; and heightened sense of well being and self-confidence along with an enhanced ability to carry out daily life activities.

The invention is illustrated further by the following examples that are not to be construed as limiting the invention in scope to the specific procedures described in them.

### EXAMPLES

### Example 1a

### 6-chloroadenosine 2′,3′-cyclohexyl ketal (58)

To a 50 mL round bottom flask were added 6-chloroadenosine (57, 1.25 g, 4.4 mmol), cyclohexanone (6.00 mL, 58.0 mmol) and trifluoroacetic acid (6.00 mL, 78.0 mmol). The mixture was allowed to stir at ambient temperature for 2 hours. The reaction was concentrated and the crude product was purified using flash chromatography to give the title compound (58) as a white solid (1.00 g, 62% yield).

### Example 1b

### 6-chloroadenosine 2′,3′-cyclohexyl ketal 5′-carboxylic acid (59)

To a 50 mL round bottom flask were added 6-chloroadenosine 2′,3′-cyclohexyl ketal (58, 320 mg, 0.9 mmol), Iodobenzene diacetate (618 mg, 1.9 mmol), acetonitrile (9 mL), water (9 mL) and 2,2,6,6-tetramethyl-1-piperidinyloxy, free radical (136 mg, 0.9 mmol). The reaction was stirred for 24 h. The mixture was poured into acetic acid and the product was extracted with ethyl acetate. The resulting solution was concentrated. Crude product 59 was used directly in the next reaction.

### Example 1c

### 6-(1-adamantylmethylamino)adenosine 2′,3′-cyclohexyl ketal 5′-carboxylic acid (4)

To a solution of 6-chloroadenosine 2′,3′-cyclohexyl ketal 5′-carboxylic acid (59, 250 mg, 0.7 mmol) in N,N-dimethylformamide (2 mL) was added 1-adamantylmethylamine. The reaction was heated at 80° C. for 24 h. The reaction was diluted with ethyl acetate/hexanes. The resulting mixture was washed with water 3 times, dried over sodium sulfate and concentrated. The crude product was purified using flash chromatography to give the title compound (4) as a white solid (80 mg, 20% yield).

### Example 2a

### Adenosine 2′,3′-cyclohexyl ketal (61)

Following the method of Example 1a, adenosine (60, 20 g, 74.8 mmol) was treated with cyclohexanone (60 mL, 579 mmol) and trifluoroacetic acid (60 mL, 779 mmol). After 1 hr at room temperature, the reaction mixture was poured into a mixture of water (300 mL), sodium bicarbonate (110 g, 1.31 mol), ice (50 g) and isopropyl acetate (100 mL). After overnight stirring, the precipitated product was filtered and washed several times with ether. Following drying 21 g of 61 was obtained (80.7% yield)

### Example 2b

### Adenosine 2′,3′-cyclohexyl ketal 5′-TBS ether (62)

Adenosine 2′,3′-cyclohexyl ketal (61, 6.0 g, 17.3 mmol) was dissolved in N,N-dimethylformamide (34 mL) and cooled on ice. Imidazole (3.53 g, 51.8 mmol) and t-butyldimethylsilyl chloride (5.21 g, 34.5 mmol) were added, the ice bath was removed and the reaction mixture stirred at room temperature for 2 hrs. The solvent was removed as well as possible via evaporation and a mixture of isopropanol (20 mL) and water (50 mL) added to the residue. Following overnight stirring, the resultant precipitate was filtered with the same ratio of isopropanol to water. Following drying the yield of the TBS ether (62) was 4.7 g (59% yield).

### Example 2c

### 6-(cyclohexylurea)adenosine 2′,3′-cyclohexyl ketal 5′-TBS ether (63) and 6-(cyclohexylurea)adenosine 2′,3′-cyclohexyl ketal (39)

Adenosine 2′,3′-cyclohexyl ketal 5′-TBS ether (62, 5.0 g, 10.8 mmol) was dissolved in dichloromethane (60 mL) in a steel bomb. Triethylamine (1.64 g, 16.2 mmol) and cyclohexyl isocyanate (4.15 mL, 32.5 mmol) were added and the sealed bombed heated for 21 hrs at 90° C. The solvent was evaporated and was replaced with tetrahydrofuran (50 mL) and cooled on ice. A 1 M solution of tetrabutylammonium fluoride in THF (12 mL, 12 mmol) was added and the solution stirred cold for 10 min followed by overnight at room temperature. The solvent was removed and the product precipitated with a mixture of water:ether. The solid was filtered, and was repurified via preparative HPLC (NovaPak C₁₈ column, 40×200 mm, gradient from 90% 0.025M ammonium acetate (pH 6)/10% acetonitrile to 100% acetonitrile over 20 min, 55 mL/min, monitor at 270 nm) Following lyophilization, the product (39) weighed 2.35 g (46% yield).

### Example 2d

### 6-(cyclohexylurea)adenosine 2′,3′-cyclohexyl ketal 5′-carboxylic acid (40)

6-(cyclohexylurea)adenosine 2′,3′-cyclohexyl ketal (39, 200 mg, 0.423 mmol) was dissolved in acetonitrile (3 mL) and water (0.5 mL). 2,2,6,6-tetramethyl-1-piperidinyloxy, free radical (66 mg, 0.423 mmol) and 6% sodium hypochlorite solution (200 uL, ca. 0.2 mmol). Several more portions of bleach solution (3×200 uL, 1×100 uL) were added over the course of 12 hrs, at which point the reaction was complete by HPLC. The reaction mixture was poured into ethyl acetate (15 mL) and 5% sodium thiosulfate solution (10 mL) and the layers separated. The organic layer was stripped and the residue reconstituted in aqueous acetonitrile. The product was purified by prep HPLC (NovaPak C₁₈ column, 40×200 mm, gradient from 70% 0.025M ammonium acetate (pH 6)/30% acetonitrile to 100% acetonitrile over 17 min, 55 mL/min, monitor at 270 nm). Following lyophilization, the product (40) weighed 90 mg (44% yield).

### Example 2e

### 6-(cyclohexylurea)adenosine 2′,3′-cyclohexyl ketal 5′-ethyl carboxamide (43) and 6-(cyclohexylurea)adenosine 2′,3′-cyclohexyl ketal 5′-(2-hydroxy)ethyl carboxamide (44)

6-(cyclohexylurea)adenosine 2′,3′-cyclohexyl ketal 5′-carboxylic acid (40, 50 mg, 0.102 mmol) was dissolved in N,N-dimethylformamide (2 mL) and 1-Hydroxybenzotriazole (14 mg, 0.103 mmol), N-(3-dimethylaminopropyl)-N′-ethylcarbodiimide hydrochloride (39 mg, 0.203 mmol) and N,N-disopropylethylamine (54 uL, 0.300 mmol) added. After 5 min, a 2 M solution of ethylamine in tetrahydrofuran (100 uL, 0.200 mmol) was added and the reaction was stirred for 2 days at room temperature. The reaction mixture was partitioned between ethyl acetate and brine, and the layers separated. The ethyl acetate was stripped and the residue reconstituted in aqueous acetonitrile. The product was purified by prep HPLC (NovaPak C₁₈ column, 20×200 mm, gradient from 90% 0.025M ammonium acetate (pH 6)/10% acetonitrile to 100% acetonitrile over 20 min, 25 mL/min, monitor at 270 nm). Following lyophilization, 20 mg amide product 43 was obtained (38% yield).

Following the protocol for the synthesis of 43, and substituting ethanolamine for ethylamine, compound 44 was obtained (15 mg, 28% yield).

### Example 3

### Calcium Signalling Protocol

### Relevance

Inhibition of Ca²⁺ influx into the cell by antagonists of the P2X₇ receptor measures inhibition of the ion channel activity of the P2X₇ receptor.

### Protocol

Human astrocytoma (1321N1) cells expressing P2X7 were grown to confluency in 96-well plates. Cells were loaded with a solution of Fluo-3 AM (2.5 μM final concentration) in an assay buffer consisting of 10 mM KCl, 118 mM NaCl, 0.25 mM CaCl₂, 1 mM MgCl₂, 20 mM HEPES, 10 mM glucose, pH 7.4. After a 60-minute incubation with Fluo-3 AM at 25° C., cells were washed and stimulated with serially diluted concentrations of compound and then challenged with 30 uM BzATP. Calcium fluorescence was measured using the FLIPR 89 (Molecular Devices Corp., Sunnyvale, Calif.) and results were calculated using Prism Graph Pad version 3.03 (San Diego, Calif.). The results are presented in Table 1. Inhibition of calcium dependent fluorescence indicates the assayed compound is an antagonist of the P2X7 receptor.

### Example 4

### IL-1β Secretion Assay Protocol

### Relevance

Inhibition of IL-1β release by antagonists of the P2X₇ receptor is a model of ameliorating inflammatory condition. This assay is an in vitro assay of cytokine secretion that can be used to evaluate the ability of compounds of this invention of Formula I to inhibit cytokine secretion, as the secretion of cytokines contributes to the inflammation in asthma, COPD and other pulmonary diseases, as well as in RA, IBD and neuropathic pain.

### Protocol

Peripheral blood from healthy human volunteers was collected in an acid-citrate-dextrose (ACD) buffer to prevent clotting, layered over a Ficoll-Paque gradient (1:1), and centrifuged for 45 minutes at 1200 rpm. The interface between the plasma layer and the Ficoll layer (buffy coat) was then diluted in RPMI 1640/10% heat inactivated FBS and centrifuged for 10 minutes at 1500 rpm. The resultant pellet was re-suspended in media containing 1 ng/mL lipopolysaccharide (LPS) and plated at a density of 500,000 cells/mL. After a 3 hour incubation (37° C., 5% CO₂, humidified air) monocytes were selected by adherence to the tissue culture plastic by washing wells with media. Following the media wash, cells were incubated for 2 minutes with the antagonists prior to the addition of 1 mM ATP. Cells were allowed to incubate with compounds for 30 minutes at 37° C. after which the supernatant was removed for immediate determination of IL-1β concentration. The concentration of IL-1β in cell supernatants was measured using the Bio-Plex (Bio-Rad) system according to manufacture's instructions and inhibition constants were calculated using Prism Graph Pad version 3.03 (San Diego, Calif.). The IC₅₀ results of the test compounds are presented in Table 1. IC₅₀ values represent the concentrations of antagonist compounds necessary to inhibit 50% of the activity (calcium dependent fluorescence or IL-1β concentration) elicited by a given concentration of ATP.

### Example 5

### Pore Formation Assay

### Relevance

Inhibition of Yo-Pro fluorescent dye influx into the cells following P2X₇ receptor activation provides a read-out of compounds potency in inhibition of pore formation activity of the P2X₇ receptor.

### Protocol

Assay was conducted essentially as in Alcaraz et al. Human astrocytoma (1321N1) cells expressing P2X7 were seeded in 96-well plates at a density of 200,000 cells/well in assay buffer consisting of 5 mM KCl, 140 mM KAspartate, 10 mM glucose, 10 mM Hepes pH 7.0, 10 mM N-methyl-D-glucoseamine, 1 mM EDTA and 1 uM Yo-Pro dye. Cells were exposed to serially diluted concentrations of compound and then challenged with 100 uM BzATP. Yo-Pro fluorescence was measured after 90 minutes. The results are presented in Table 1. Inhibition of Yo-Pro dependent fluorescence indicates the assayed compound is an antagonist of the P2X7 receptor.

TABLE 1

| | | Inhibition of | |
|---|---|---|---|
| | IL1β inhibition | Ca⁺² signaling | Pore formation assay |
| COMPOUND | IC₅₀ (nM) | IC₅₀ (nM) | IC₅₀ (nM) |
| 38 | 98 | 597 | 52 |
| 39 | 65 | 477 | 346 |
| 43 | 69 | 295 | 152 |
| 44 | 58 | 404 | 257 |
| 46 | 99 | 852 | 234 |

### Example 6

### Inhibition of Neutrophilia and Pro-Inflammatory Cytokine Secretion in Inhaled LPS-Exposed Mice

### Relevance

The exposure of experimental animals to aerosolized LPS leads to acute inflammatory response in the pulmonary tissue accompanied by pro-inflammatory cytokine secretion and neutrophil accumulation. These inflammatory processes are common to the diseases targeted for treatment with the compounds of this invention.

### Protocol

Lipopolysaccharide (LPS), a major proinflammatory glycolipid component of the gram-negative bacterial cell wall, is one of the agents ubiquitously present as contaminant on airborne particles, including air pollution, organic dusts, and cigarette smoke. Chronic exposure to significant levels of LPS is reported to be associated with the development and/or progression of many types of lung diseases, including asthma, chronic bronchitis, and progressive irreversible airflow obstruction, that are all characterized by chronic inflammatory processes in the lungs.

Male BALB/c mice were ordered from Charles River Laboratories (Raleigh, N.C.). The animals were approximately 19 to 21 grams at time of receipt at the RTI animal research facility (ARF). Upon arrival to the facility, the animals were randomized into groups of five males per cage and assigned to a dosing group. Animals were quarantined for 7 days under test conditions. They were observed daily for general health status and ability to adapt to the water bottles.

All animals were challenged with aerosolized LPS at 10 ug/ml concentration for 25 minutes on study day 0. Aerosol challenge consists of using an Aerogen Aeroneb nebulizer and controller with a particle size of 2-4 μm mass median aerodynamic diameter (MMAD) with a distribution of 400 μl per minute.

Compounds of this invention were dosed to animals 1 hour prior to the LPS challenge (compound #38 p.o. and i.p.; compound #44 i.p. and intratracheal [i.t.]). Four hours after the LPS challenge, a bronchoalveolar lavage fluid (BALF) was collected by infusing first 0.5 mls followed by 2.5 mls for a total of 3.0 ml of saline with 10% fetal calf serum into the lungs via the trachea and then withdrawing the fluid. The first 0.5 ml BALF was centrifuged, supernatant removed and frozen for cytokine analysis, and cell pellet reconstituted in the remaining 2.5 mls BALF. This sample was centrifuged, supernatant removed, and cell pellet reconstituted in 500 ul of fluid. Cytospin slides prepared from the cell pellet using 100 ul of fluid and spinning samples for 5 minutes at 5000 rpms in a cytospin centrifuge. Following Hema3 stain, a differential evaluation of cellular content was determined on a 200 cell count.

The concentrations of IL-1B in the BALF samples were determined using commercially available Bio-plex kits (Bio-Rad) for the detection of mouseIL-1B. The analysis of cytokine levels was measured using the Bio-Plex 200 (Bio-Rad) system according to the manufacturer's instructions.

### Results

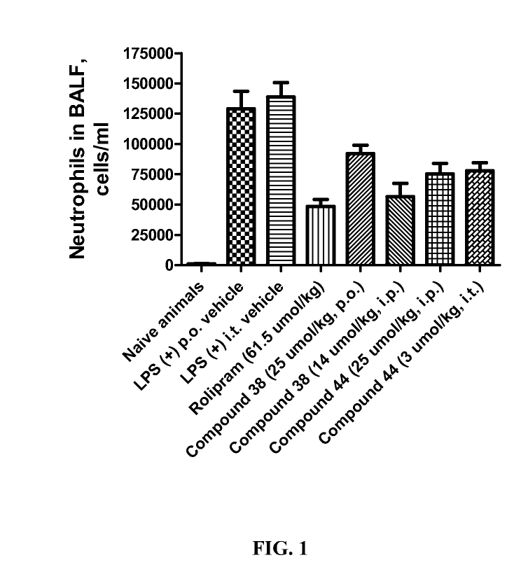

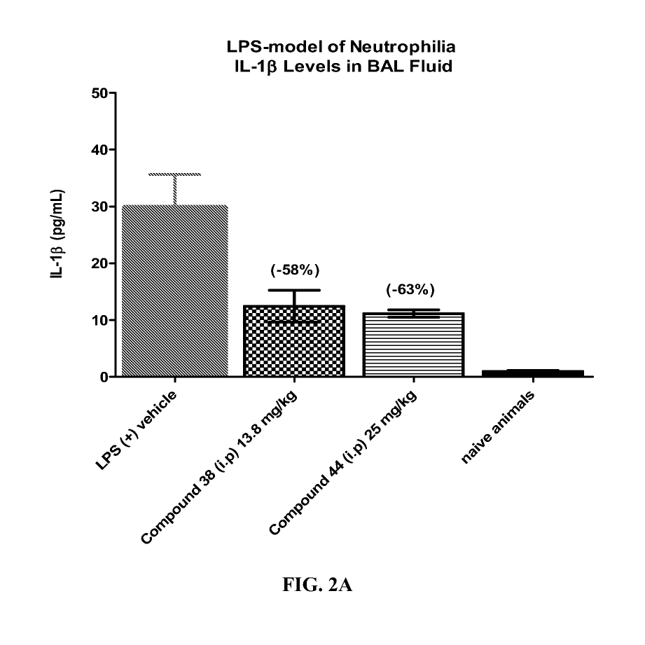

The cellular content of the BALF was evaluated, total cell counts were increased in the LPS animals compared to naive animals. Evaluation of the differential counts performed on these samples showed an increased number of inflammatory cells in the LPS-exposed animals. Airway neutrophil infiltration was reduced in the naïve animals and those treated with compounds of this invention (FIG. 1).

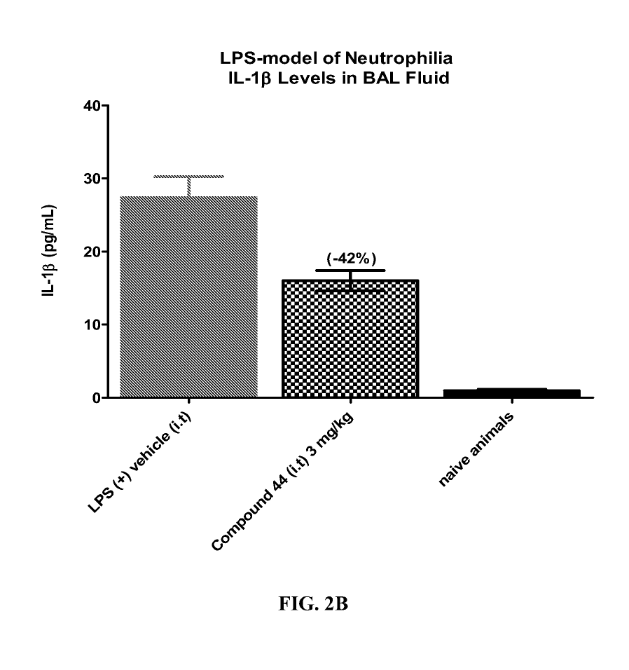

Similarly, the levels of IL-1B in the BALF were reduced following either i.p. or intra-tracheal (i.t.) dosing of compounds of this invention (FIGS. 2A and 2B).

### Example 7

### Efficacy of Compounds in Animal Model of Idiopathic Pulmonary Fibrosis

This example illustrates the efficacy of compounds of this invention in treatment of IPF in bleomyocin-induced pulmonary fibrosis in mice

### Protocol

The model is based on the description in Shimizu Y et al. Am. J. Resp. Crit. Care Med. 163: 210-217, 2001. Pathogen-free 6-wk-old female C57BL/6 mice are used for the experiments. The animals are maintained under standard conditions with free access to water and rodent laboratory food. The animals receive bleomyocin (BLM) i.p. injections on day 0, 2, 4, 6 and 8 at a dose of 40 mg/kg. BLM accumulates in the subpleural regions, resulting in the preferential development of lung fibrosis at subpleural lesions. This is very similar to the pathological features of human IPF (Ekimoto H et al. Gan To Kagaku Ryoho 10:2550-2557, 1983). Body weights are measured before every administration of the compounds. Compounds of formula I are administered via i.p. administration every day at the dose of 1 mg/kg to 100 mg/kg of body weight starting on day 0 and continuing to day 40. A control group of animal receives i.p. saline.

At day 40, mice are sacrificed, and their thoraces are then exposed. The lungs are washed with cold phosphate-buffered saline (PBS) and surgically removed. The excised lungs are used for histopathological examination and assayed for OH-proline contents. The left lungs are used to evaluate the fibrotic score by histological examination, and the right lungs for measurement of OH-proline contents. Additional mice are used to determine cell differentiation in the lumen of the lung as determined by bronchoalveolar lavage (BAL). BAL is performed on Days 7, 14, 21, and 40 after initial injection of BLM. Mice are sacrificed, and BAL is performed.

### Histologic Examination

Morphological evaluation of fibrotic changes in the lungs is performed on Day 40. The excised lungs are immediately fixed with 10% formaldehyde neutral buffer solution for 48 h, and then embedded in paraffin. Sagittal sections are cut at 2 mm thickness and stained with hematoxylin-eosin and Masson-trichrome. The total lung area of the sections is used for the fibrotic scale microscope evaluation (Olympus, B X50F4). Criteria for grading lung fibrosis are according to the method reported by Ashcroft and coworkers (Ashcroft T et al. J Clin Pathol. 41:467-470, 1988): Grade 0, normal lung; Grade 1, minimal fibrous thickening of alveolar or bronchiolar walls; Grade 3, moderate thickening of walls without obvious damage to the lung architecture; Grade 5, increased fibrous with definite damage to lung architecture and formation of fibrous bands or small fibrous masses; Grade 7, severe distortion of architecture and large fibrous area; Grade 8, total fibrous obliteration of the field. Severity of fibrotic changes in each lung section is assessed as the mean score for severity from the observed microscopic fields. The grade of lung fibrosis is scored on a scale from 0 to 8 by examining 20 randomly chosen regions per sample at a magnification of 3100. To minimize investigator variability, all histological specimens are randomly numbered and scored by another investigator in a single blinded fashion.

### OH-Proline Assay

OH-proline contents of the lungs are measured objectively to estimate lung fibrosis (Green G D et al. Anal Biochem. 201:265-269, 1992). The right lungs of each mouse are dissected free from major bronchi, and the wet weights are measured. They are hydrolyzed in 500 ml of 12 N hydrochloric acid and in the same aliquot of distilled water at 110 C 20 h, in dry block. After the resultant hydrolysate is neutralized with sodium hydroxide, a 100-ml supernatant is mixed in 1.5 ml of 0.3 N lithium hydroxide solution. The OH-proline content is determined by high-performance liquid chromatography (HPLC) and expressed as micrograms per right lung.

### Bronchoalveolar Lavage and Cell Counting

Bronchoalveolar lavage fluid (BALF) is collected by infusing 3.0 ml of saline with 10% fetal calf serum into the lungs via the trachea and then withdrawing the fluid. The total amount of cells/ml of BALF fluid is determined via manual cell count on hemocytometer. The BALF is centrifuged, and cell pellet reconstituted in 500 μl, of fluid. Cytospin slides are prepared from the cell pellet using 100 μL of fluid and spinning samples for 5 minutes at 5000 rpms in a cytospin centrifuge. Following Hema3 stain, relative percentages of individual leukocytes are determined on a 200 cell count for each sample. The final concentration of individual leukocyte cell types per ml of BALF is determined by multiplication of the relative percentage of individual leukocytes with the total amount of cells/ml of BALF fluid.

### Results

At day 40 following the first BLM administration, the fibrotic changes in the lung, the hydroxyproline content in the lung, and the cell count of leukocytes (total cell count, macrophage cell count, lymphocyte cell count and/or nertrophil cell count) in BALF are measured and compared in the compound-treated mice vs. saline-treated mice. Improvement in at least one of the above-mentioned endpoints is observed.

### Example 8

### Efficacy of Compounds of Formula I in Treating RSV-Infection Induced Inflammation and Airway Hyperresponsiveness

### Protocol

The experiment is conducted essentially as in Hashimoto K et al. Thorax, 57:524-527, 2002. In summary, ovalbumin (OVA) sensitized mice, which are also RSV infected, demonstrate prolonged methacholine-induced airway hyperresponsiveness (AHR) when compared to OVA sensitized mice without RSV infection. According to past observations, ovalbumin (OVA)-induced AHR lasted only a few days past the discontinuance of OVA aerosol in mice that were ovalbumin sensitized and mock infected. In contrast, OVA-sensitized mice infected with RSV during the OVA aerosol treatments (OVA/RSV) had AHR for more than 2 weeks after infection (Peebles R S et al. J. Med. Virol. 57 (2):186-92, 1999).

Pathogen free 8 week old female BALB/c mice are used. The A2 strain of RSV virus is prepared as previously described in Graham B S et al. J Med Virol 26:153-62, 1998. Mice are injected intraperitoneally with 0.1 ml (10 μg) ovalbumin complexed with 2 mg Al(OH)₃ as previously described (Peebles R S et al. J. Med. Virol. 57 (2):186-92, 1999). After 14 days, the mice are placed in an acrylic box and exposed to aerosols of 1% ovalbumin diluted in sterile phosphate buffered saline (PBS) using a nebulizer for 40 minutes each day for 8 days. Mice are infected with RSV (as previously described on day 3 of OVA inhalation (Peebles R S et al. J. Med. Virol, 57 (2):186-92, 1999). Fourteen days after RSV inoculation (and 9 days after OVA inhalation), the mice undergo AHR testing via methacholine challenge. The mice are administered with Formula I compound i.p. at 1-100 mg/kg of body weight. AHR is measured one hour after the treatment (Peebles R S et al. J. Med. Virol. 57 (2):186-92, 1999).

### AHR Measurements

AHR is measured as previously described (Peebles R S et al. J. Med. Virol. 57 (2):186-92, 1999). Methacholine is dissolved in normal saline and administered intravenously at starting doses of 5 μg/kg and 6.25 μg/kg, respectively. The mean volume per methacholine dose is approximately 35 μl and 50 μl respectively. The methacholine concentration is increased in multiples of three in the dose response challenge with methacholine.

### Bronchoalveolar Lavage and Cell Counting

Bronchoalveolar lavage fluid (BALF) is collected by infusing 3.0 ml of saline with 10% fetal calf serum into the lungs via the trachea and then withdrawing the fluid. The total amount of cells/ml of BALF fluid is determined via manual cell count on hemocytometer. The BALF is centrifuged, and cell pellet reconstituted in 500 μL of fluid. Cytospin slides are prepared from the cell pellet using 100 μL of fluid and spinning samples for 5 minutes at 5000 rpms in a cytospin centrifuge. Following Hema3 stain, relative percentages of individual leukocytes are determined on a 200 cell count for each sample. The final concentration of individual leukocyte cell types per ml of BALF is determined by multiplication of the relative percentage of individual leukocytes with the total amount of cells/ml of BALF fluid.

### In Vitro Assessment of Cytokine and Chemokine Levels

Supernatant retained from the bronchoalveolar lavage is analyzed for concentrations of proinflammatory cytokines and chemokines including but not limited to the following: Il-1beta, IL-1alpha, TNF-alpha, TNF-beta, RANTES, IL-6, IL-8, IL1-11, GM-CSF, MIP-1-alpha, MIP-1-beta, MCP1, MCP2, MCP3 and MCP4. The concentrations of these cytokines and chemokines in the BALF samples are determined using commercially available kits.

### Results

Airway hyperresponsiveness and airway inflammation is measured as described above. Improvement in at least one of the above-mentioned endpoints is observed.

### Example 9

### Prevention of Acute Lung Injury by Compounds of Formula I or II in an Oleic-Acid Rat Model of ARDS

The aspiration of stomach contents into the lungs during obstetric anesthesia (Am J Obstet Gynecol 1946; 52:191) and aspiration of gastric contents is now recognized as an important risk factor for ARDS (Pepe P E et al. Am J Surg, 144:124-30, 1982; Hudson L D et al. Am J Respir Crit Care Med, 151:293-301, 1995; Doyle R L et al. Am J Respir Crit Care

Med, 152:1818-24, 1995). Oleic acid-induced lung injury is a well established model of ARDS (Dickey B F et al. Am. J. Pathol., 103:376-383, 1981). It is characterized by diffuse interstitial and alveolar edema with focal hemorrhage and vascular congestion, and by interstitial and alveolar infiltration of leukocytes (Beilman G. Lipids 30:817-823, 1995).

Both sexes of Wistar rats are randomly separated into treatment groups: untreated control, oleic acid-treated control, oleic acid plus a Compound of Formula I or II, untreated plus a Compound of Formula I or II. All oleic acid treated animals receive a single intravenous (i.v.) administration while untreated animals receive a single i.v. administration of saline. Oleic acid and saline are injected into the tail vein under light anesthesia with ketamine. Acute lung injury is induced by intravenous administration of 100 mg/kg of oleic acid (cis-9-octadecanoic acid). Oleic acid is initially diluted in ethanol and saline is added to a final concentration of 25 mg/ml of oleic acid. A Compound of Formula I or II is administered at a dose from 1 to 100 mg/kg either orally, intravenously, intraperitoneally, intracheally or intranasally. Animals receive drugs or saline four hours prior to necropsy.

Four hours after the administration of the drugs, the rats are anaesthetized with a high dose of ketamine (80 mg/kg, i.m.), the thorax is opened and blood samples are taken by cardiac puncture for malondialdehyde, myeloperoxidase, 3-nitro-1-tyrosine and nitrite/nitrate analysis (as markers of lung injury). Thereafter, both lungs are harvested. Some pieces of lungs are preserved in formaldehyde solution (10%) for histopathologic evaluation. Haematoxylin-eosin-stained slides are prepared using standard methods. Other lung pieces are used for biochemical examination and Western blotting.

In oleic acid only treated animals, pronounced acute lung damage is observed. The lung tissue is much darker red in the oleic acid group than in the other groups. Furthermore, an increase in congestion, neutrophil infiltration and even derangement of pulmonary architecture is observed under light microscopy. Increases in serum and tissue nitrite/nitrate, 3-nitro tyrosine, myeloperoxidase and malondialdehyde levels are also observed.

Administration of a compound of Formula I or II causes a significant improvement in at least one of the following parameters: lung histology with score(s) assessing lung tissue damage, inflammation, and edema; gross appearance of the lung including the color of the lung similar to that in the sham group; normalization of serum nitrite/nitrate, myeloperoxidase and malondialdehyde or tissue 3-nitro tyrosine, myeloperoxidase or malondialdehyde levels.

### Example 10

### Attenuation of Microvascular Leak in Rat Model of VILI

Microvascular leak is one of the defining features of the ARDS and VILI. Male Sprague-Dawley rats are anesthetized intraperitoneally with ketamine and diazepam. Rats are ventilated with room air at 85 breaths/minute for 2 hours either with a ventilation (VT) of 7 ml/kg (VT7) or 20 ml/kg (VT20) and zero end expiratory pressure. A group of animals with a VT of 20 ml/kg receives 10 ml/kg of normal saline (NS) to correct hypotension related to large VT (VT20NS). Airway pressure and systemic arterial pressure are monitored. A compound of Formula I (1-100 mg/kg) is given intraperitoneally 30 minutes before starting mechanical ventilation.

After 90 minutes of mechanical ventilation, an intravenous injection of 30 mg/kg Evans Blue Dye (EBD) (Sigma Chemical) is given through the internal jugular vein. EBD extravasation into the lung parenchyma as an estimate of protein permeability is quantitated as previously described (Green T P et al. J Lab Clin Med, 111:173-183, 1988). EBD leak in the lung is significantly higher in VT20 and VT20NS groups compared with the VT7 group. There is no significant difference in EBD leak between VT20 and VT20NS. After administration of a compound of Formula I, an improvement in at least one of the following parameters is observed: EBD leak in the lung is decreased in VT20 (+) Compound of Formula I and/or VT20NS (+) Compound of Formula I groups compared with VT20 and VT20NS groups; and/or lung weight is significantly higher in VT20 and VT20NS groups compared with VT7 and compound of Formula I attenuates the increase in lung weight in the large VT groups.

### Example 11

### A Randomized Trial of a Compound of Formula I in Patients with ARDS

With the assent of the attending physician, informed consent is obtained from the patient or next of kin as soon as possible after case identification. Physiologic measurements and specimen collection begins at the time of entry into the study. Three days after the patient has met criteria for ARDS or at entry into the study (whichever is later), he/she is randomized to receive a Compound of Formula I (0.5-50 mg/kg) or placebo, administered by intravenous infusion or directly into the lumen of the lung once daily for 14 days.

The primary endpoint for this study is the duration of mechanical ventilation. Additional important endpoints include changes in the severity of physiologic derangements of respiratory gas exchange, non-respiratory organ failure, and incidence of ventilator-associated pneumonia. Additional assessments designed to determine the mechanism of benefit of Compound of Formula I include measures of lung epithelial cell integrity and measures of alveolar macrophage (lung inflammatory cell) function. It is observed that the administration of a Compound of Formula I improves ARDS by the improvement of any of the primary or secondary endpoints measured in this study.

### Example 12

### Efficacy of Compounds of Formula I in Reduction of Pulmonary Inflammation Relevant to CF

Relevance. CF lung disease is characterized by pulmonary inflammation, airway hyperreactivity, and pulmonary fibrosis, and anti-inflammatory drugs are important therapeutic agents in the treatment of CF (Schmitt-Grohé S and Zielen S._{—}Paediatr Drugs. 7 (6):353-63, 2005; Elizur A et al. Chest 133 (2):489-95, 2008). Airway eosinophil infiltration plays a role in CF pathogenesis (Schmitt-Grohé S and Zielen S._{—}Paediatr Drugs. 7 (6):353-63, 2005). CF lung disease also involves infiltration of polymorphonuclear leukocytes (Elizur A et al. Chest 133 (2):489-95, 2008). Therefore, the anti-inflammatory efficacy of Compounds of Formula I as described in Examples 4, 5 and 6 demonstrate therapeutic utility of these compounds in treatment of CF.

### Example 13

### Efficacy of Compounds of Formula I in Reduction of Pulmonary Inflammation, Airway Hyperreactivity, Bronchoconstriction, Pulmonary Permeability and Edema Accompanying Bronchiectasis

Pulmonary inflammation is a key pathophysiology accompanying bronchiectasis. Therefore, the anti-inflammatory efficacy of Compounds of Formula I as described in Examples 6, 7, 8, 9, and 10 demonstrate therapeutic utility of these compounds in treatment of bronchiectasis. Bronchoconstriction and airway hyperreactivity is a key pathophysiology accompanying bronchiectasis. Therefore, efficacy of Compounds of Formula I as described in Examples 4, 5, and 6 demonstrates therapeutic utility of these compounds in treatment of bronchiectasis. In addition, the following examples illustrate the efficacy of Compounds of Formula I in reduction of LPS induced pulmonary permeability in rats and LPS-induced airway wall thickening in mice.

### Protocol

Model is prepared essentially as in Eutamene et al Eur. Resp. J., 25 (5):789-796, 2005. Male Wistar rats are anaesthetized using pentobarbital (60 mg/kg body weight-1 intraperitoneally) and anesthesia is maintained with half of this dose 2 h later. An endotracheal cannula equipped with a small catheter is inserted through a tracheotomy. For experiments using LPS from P. aeruginosa or vehicle (sterile 0.9% NaCl), an iso-osmolar solution is prepared, containing 5% bovine serum albumin in phosphate-buffered saline. The solution is filtered through a 0.2-mm filter and 0.5 mCi iodine-125-labelled human serum albumin ([125I] albumin) is added to the bovine serum albumin solution. Then LPS from P. aeruginosa (1 mg/rat-1) or vehicle is added to the instillate immediately prior to instillation into the trachea at a constant rate of 10 mL/min-1 for 15 min. Four hours after tracheal infusion of [¹²⁵I]-albumin labeled alveolar instillate plus LPS, radioactivity is measured in three compartments: plasma, lung airspace (via bronchoalveolar lavage (BAL)), and total lung tissue. For the evaluation of pulmonary permeability, rats are pretreated twice daily for 2 days with the Compounds of Formula I (first bolus administered i.p. or p.o. at 1-100 mg/kg body weight and successive administrations at 1-100 mg/kg body weight) or vehicle (0.2 mL 10% ethanol). The last administration of kinase inhibitor or vehicle is performed 1 h before intratracheal infusion of LPS from P. aeruginosa. Four hours after LPS infusion, measurements of epithelial permeability are performed. Evaluations of airway epithelial barrier (AEB) permeability required measurement of residual [¹²⁵I]-albumin, the airspace protein tracer, in the lung, as well as accumulation of [¹²⁵I]-albumin in the plasma. Four hours after infusion of LPS from P. aeruginosa, residual [¹²⁵I]-albumin is measured in BAL fluid, lung tissue (after lavage) and plasma. Plasma [¹²⁵I]-albumin levels are measured in abdominal aorta blood samples. The plasma fraction is determined by multiplying the number of counts obtained by the plasma volume (0.07 body weight (1-haematocrit)). All of these residual counts (BAL fluid, lung tissue and plasma) are expressed as a percentage of the total number of counts of [¹²⁵I]-albumin administered intratracheally (100%).

### Results

Intratracheal infusion of LPS from P. aeruginosa enhances airway epithelial paracellular permeability to large molecules, and the percentage of [¹²⁵I] collected in lung tissue is significantly increased in LPS-treated rats compared to controls. In contrast, levels of [¹²⁵I] in BAL fluid are decreased in LPS animals compared to controls, confirming the increase in albumin passage from the airspace to lung tissue. Pretreatment with the Compounds of Formula I reduces the increase in lung epithelial permeability induced by LPS and/or the Compounds of Formula I restore [¹²⁵I] levels in BAL fluid from LPS-treated rats to values closer to controls.

### Example 14

### Efficacy of Compounds of Formula I in Reduction of Pulmonary Remodeling Accompanying Bronchiectasis

The following example illustrates the efficacy of Compounds of Formula I in treatment of bronchiectasis in mouse model of LPS induced airway wall thickening. Model is prepared essentially as in (Vernooy et al., Am. J. Respir. Cell Mol. Biol., 26:152-159, 2002.)

### Protocol

Male Swiss mice 12 week old are used. Animals are housed individually in standard laboratory cages and allowed food and water ad libitum throughout the experiments. Mice are repeatedly challenged with LPS twice a week for a period of 12 weeks by intratracheal instillation in an attempt to induce a chronic pulmonary inflammation. The dose of LPS used is approximately 5 μg/instillation/mouse. Sham mice are instilled intratracheally with LPS-free sterile 0.9% NaCl, whereas control mice receive no treatment. Intratracheal instillation is performed by a nonsurgical technique. In brief, mice are anesthetized by intraperitoneal injection of xylazine/ketamine. A volume of 50 μL is instilled intratracheally via cannula, followed by 0.1 ml of air. After intratracheal treatment, the mice are kept in an upright position for 10 min to allow sufficient spreading of the fluid throughout the lungs. The Compounds of Formula I are administered i.p. or p.o. at 1-100 mg/kg body weight daily starting with the first LPS administration into the animals over the course of the 12 weeks.

Airway wall thickening is determined using standard morphometric technique on alpha-SMA stained paraffin section cut from the upper part of the left lung. Conducting airways (width>190 μm) are captured at 20× with a digital camera and the smooth muscle cell area surrounding the airways is quantified by computerized morphometry using the an imaging analysis system. Increased width of the smooth muscle layer is taken as evidence of airway wall thickening. Standard morphometric technique is used to determine the presence of emphysematous changes in the lungs. In brief, H&E stained paraffin sections cut from the upper part of the left lung are used, and 10 randomly selected fields are sampled by projecting a microscopic image of the lung section on a screen with a square reference lattice containing one horizontally and one vertically placed test line. The number of intersections of alveolar walls on the test lines are quantified by computerized morphometry using an imaging analysis system and used to quantify alveolar mean linear intercept (LM, the average distance between alveolar walls). Increased LM was taken as evidence of alveolar enlargement.

### Results

Treatment of LPS-exposed animals with Compounds of Formula I results in reduced airway wall thickening or decreases in LM during at least one of the time-points over the 12-week LPS exposure when compared to LPS-exposed untreated animals over the same time period.

### Example 15

### Efficacy of Compounds of Formula I in Reduction of Pulmonary Inflammation Relevant to AATD

Relevance. AATD lung disease involves pulmonary inflammation, airway hyperreactivity, and pulmonary fibrosis, and anti-inflammatory drugs are important therapeutic agents in the treatment of AATD. Therefore, the anti-inflammatory efficacy of Compounds of Formula I as described in Examples 4, 5 and 6 demonstrate therapeutic utility of these compounds in treatment of AATD.

### Example 16

### Efficacy of Compounds of Formula I in an Animal Model of Rhinitis

Nasal congestion due to inflammation and tissue edema is one of the key pathophysiologies defining rhinitis. In the following dog model of ragweed-induced rhinitis, nasal congestion is measured via acoustic rhinometry and nasal resistance.

### Protocol

Newborn dogs receive an intraperitoneal injection containing 200 μg of ragweed extract in 0.5 ml of 0.9% saline mixed with 30 mg of aluminum hydroxide within 24 hours of birth. (Becker et. al. J Appl Physiol. 1989. 66:2691-2697; Yeates et. al. Proc Assoc Am Physicians. 1997, 109: 440-52). Booster injections are repeated weekly for 6 weeks and biweekly until 16 weeks of age. Sensitization to the allergen is confirmed by analysis of ragweed-specific IgE levels in the serum of the animals. For the experiment, fasted dogs are anesthetized and intubated. A nasal catheter is placed in each nostril to facilitate measurements of airway resistance. Nasal congestion in ragweed-sensitized dogs is induced by local, acute administration of histamine as the challenging allergen. Acoustic rhinometry and nasal airway resistance are measured between 4 and 24 hr post histamine administration to evaluate benefit of formulated compounds (Tiniakov et al. J Appl Physiol 2003. 94: 1821-1828).

Compounds of Formula I are dosed via bilateral intranasal administration at 30-60 minutes before histamine challenge at a dose volume of 100 μL per nostril at a concentration range of 10 μM to 10 mM range. A control group receives bilateral nasal administration of vehicle (placebo) at the same administration volume as active.

### Acoustic Rhinometry

Nasal resistance can be measured in both the right and left nasal passages by using an anterior constant flow nasal rhinomanometry device. Changes in the geometry of the nasal cavity can be estimated using Acoustic Rhinometry System. The acoustic wave tube is fitted with a handmade plastic tip designed to match to the shape of the dog's nostrils. Acoustic measurements of the geometric parameters of the right nasal passage are performed at various times after allergen or constricting agent is applied. Volume of the right nasal airway and cross-sectional areas of right nasal cavity at the levels of a nasal valve, anterior and posterior regions of maxilloturbinates, and the moturbinates can be calculated using acoustic rhinometry.

### Measurement of Nasal Resistance

Airway resistance can be measured in combination with acoustic rhinometry. Nasal airways resistance is determined by measuring the air pressure required to achieve a constant predetermined flow through the nasal passage. This constant airflow is delivered to the nasal passage through a nasal catheter coupled to a pressure transducer. The nasal catheter is snugly placed into the nostril and the cuff inflated to form a seal. Nasal resistance is defined as the pressure differential between the input air pressure and atmospheric pressure divided by the airflow. In these studies, nasal resistance can be measured in the left nasal airway and geometric parameters of the right nasal airway are measured with the acoustic rhinometer, simultaneously. To do this, allergen or constricting agent is locally delivered to both nasal passages.

### Results

Between 4 and 24 hours following nasal administration of histamine, animals are evaluated for acoustic rhinometry and nasal resistance. Improvement in either acoustic rhinometry or nasal resistance is observed between 4-hr to 24-hr in animals dosed with a Compound of Formula I when compared to animals that receive placebo.

### Example 17

### Efficacy of Compounds of Formula I in Attenuating Pathophysiologies Associated with Rhinosinusitis

The following example illustrates the efficacy of compounds of this invention in treatment of inflammation in mouse model of sinusitis. Model is prepared essentially as in Blair, C., et al. J. Allergy Clin. Immunol, 108 (3):424-9, 2001.

### Protocol

Pathogen-free 6 to 8-week-old BALB/c mice of either sex are used. Each group of animals is kept isolated from the other groups in a biohazard containment facility. All mice use is in accordance with National Institutes of Health Laboratory Animal Care Guidelines.

A group of animals is pretreated with a compound of Formula I via intra-peritoneal administration b.i.d. at 1-100 mg/kg of body weight on Day 1-3 and one hour prior to inoculation on day 4 while the control group is dosed with vehicle. S. pneumoniae (ATCC49619) is used for induction of acute sinusitis. The strain is antigenically similar to type 19 S. pneumoniae, the most common strain cultured from human sinuses. The S. pneumoniae is grown on blood agar plates, and colonies are suspended in sterile saline solution immediately before inoculation of the mice. The mice are anesthetized with intraperitoneal injection of ketamine/xylazine, and sufficient amount of the S. pneumoniae suspension is placed in each naris to induce infection. The mice are killed on day 5 after infection; prior experiments have shown peak infection in the sinuses at that time point.

On the day of sacrifice, the mice are sedated with a respiratory-failure dose of pentobarbital sodium (Nembutal) at 120 mg/kg. The animal is transcardially (through the right atrium) perfused with lactated Ringer's solution; this is followed by perfusion with a solution of 4% formaldehyde and 0.5% glutaraldehyde in 0.1 mmol/L of phosphate buffer. Next, the animal is decapitated and sections of the nasal passages are cut at a thickness of 8 μm, mounted on glass slides, and stained with Luna stain or hematoxylin and eosin.

### Light Microscopy and Enumeration of Inflammatory Cells

Three anatomically similar sections are chosen from each mouse for analysis: an anterior section at the level of the maxillary sinuses, a middle section (more posterior and sampling the end of the maxillary sinuses and the beginning of the ethmoidal turbinals), and a third posterior section. Individual sections are analyzed, after masking, by use of a computer-aided light microscope in conjunction with reconstruction software. To quantify the degree of inflammation, we use 400× magnification and trace the total sinus cavity area and the area of the sinus occupied by neutrophil clusters; this allows us to calculate the percent of the sinus cavity filled with neutrophil clusters. Mucosa adjacent to neutrophil clusters is also traced and examined for polymorphonuclear cells, allowing us to report the number of cells per square millimeter. A random sampling of 4 mucosal areas from each of the 3 sections from each mouse is evaluated for the parameters described above, and the average of these measurements is computed for each mouse and used for statistical analysis. Eosinophils and mononuclear cells, as well as eosinophils in the lung, are counted in similar manner.

### Results

The resulting inflammatory cell counts demonstrate that treatment with a Compound of Formula I attenuates the inflammatory cell numbers identified in the nasal passage ways of mice with experimental sinusitis when compared to the non-treated animals with experimental sinusitis.

### Example 18

### Efficacy of Compounds of Formula I in Reduction of Pulmonary Inflammation Relevant to OB/BOOP Due to Lung Transplantation or HSCT

Relevance. OB/BOOP involves pulmonary inflammation, airway hyperreactivity, and pulmonary fibrosis, and anti-inflammatory drugs are important therapeutic agents in the treatment of OB/BOOP. Therefore, the anti-inflammatory efficacy of Compounds of Formula I as described in Examples 4, 5, 6, 7, 9, 10 and 11 demonstrate therapeutic utility of these compounds in treatment of OB/BOOP.

### Efficacy of Compounds of Formula I in an Animal Model of BOOP

The following example illustrates the efficacy of Compounds of Formula I in treatment of BOOP in an animal model of virally induced intraluminal fibrosis. The model is prepared essentially as in Majesky et al., Am J Pathol, 163:1467-1479, 2003.

### Protocol

Four- to 5-week-old female CBA/J mice are lightly anesthetized and infected by the intranasal (i.n.) application of 1×10⁶ PFU of reovirus 1/L in 30 ml (15 ml in each nostril) in sterile saline on day 0. Control animals are inoculated with sterile saline alone. A compound of Formula I is administered to mice beginning on day 5 post-reovirus 1/L infection and given daily until the completion of the time-course. As a control standard compound, 10 mg/kg of methylprednisolone is administered i.p. to mice beginning on day 5 post-reovirus 1/L infection and given daily until the completion of the time-course. On days 7, 10, and 14 BAL fluid is taken for measurement of cytokines. On day 14 or day 21, animals are sacrificed for histological evaluation of the lung.

### Cytokine Determination in BAL Fluid

BAL is performed in situ by injecting and withdrawing a 0.5 ml aliquot of Hank's balanced salt solution (HBSS) twice through an intubation needle (21 gauge). BAL fluid is analyzed for mouse IFN-γ and MCP-1 using commercially available ELISA kits.

### Histology

On day 14 or 21, lungs are inflated in situ with 10% neutral buffered formalin (0.5 mls) by intratracheal (i.t.) intubation, removed, and suspended in an additional 10% neutral buffered formalin overnight before being embedded in paraffin. H&E stain and Mason's trichrome stain, which are used to visualize collagen deposition, are performed on 4-μm sections. Inflammatory infiltration with the development of follicular bronchiolitis (FB), which is defined as a mononuclear cell infiltrate that condenses into prominent peribronchiolar lymphoid accumulations, is blindly evaluated. FB is scored on a scale of 0 to 3: 0, normal; 1, mild (less than 4 follicles per lobe); 2, moderate (between 5 and 8 follicles per lobe); 3, severe (greater than 8 follicles per lobe). Fibrosis is scored on a scale of 0 to 4: 0, normal; 1, mild; 2, moderate; 3, severe; 4, very severe.

### OH-Proline Assay

On day 14 or 21, OH-proline contents of the lungs are measured objectively to estimate lung fibrosis (Green G D et al. Anal Biochem. 201:265-269, 1992). The right lungs of each mouse are dissected free from major bronchi, and the wet weights are measured. They are hydrolyzed in 500 ml of 12 N hydrochloric acid and in the same aliquot of distilled water at 110 C 20 h, in dry block. After the resultant hydrolysate is neutralized with sodium hydroxide, a 100-ml supernatant is mixed in 1.5 ml of 0.3 N lithium hydroxide solution. The OH-proline content is determined by high-performance liquid chromatography and expressed as micrograms per right lung.

### Results

On the indicated day, the fibrotic changes in the lung, the hydroxyproline content in the lung, and the cytokine content in the BAL fluid are measured and compared in the compound-treated mice vs. saline-treated mice. Administration of Compounds of Formula I result in the improvement in at least one of the above-mentioned endpoints that is equal to or greater than the improvement seen with methylprednisolone.

### Example 19

### Efficacy of Compounds of Formula I in Attenuating Pathophysiologies Relevant to Non-IPF IIP

### Efficacy of Compounds of Formula I in Reduction of Pulmonary Inflammation Relevant to Non-IPF IIP

Relevance. Non-IPF IIP involves pulmonary inflammation, airway hyperreactivity, and pulmonary fibrosis, and anti-inflammatory drugs are important therapeutic agents in the treatment of non-IPF IIP. Therefore, the anti-inflammatory efficacy of Compounds of Formula I as described in 4, 5, 6, 7, 9, 10, 11 and 18 demonstrate therapeutic utility of these compounds in treatment of non-IPF IIP.

### Example 20

### Efficacy of Compounds of Formula I in Attenuating Pathophysiologies Relevant to the ILD Other than IPF, Non-IPF IIPs and OB/BOOP

Efficacy of Compounds of Formula I in Reduction of Pulmonary Inflammation Relevant to ILD Other than IPF, Non-IPF IIPs and OB/BOOP

Relevance. ILD other than IPF, non-IPF IIPs and OB/BOOP involves pulmonary inflammation, airway hyperreactivity, and pulmonary fibrosis, and anti-inflammatory drugs are important therapeutic agents in the treatment of ILD other than IPF, non-IPF IIPs and OB/BOOP. Therefore, the anti-inflammatory efficacy of Compounds of Formula I as described in Examples 4, 5, 6, 7, 9, 10, 11 and 18 demonstrate therapeutic utility of these compounds in treatment of ILD other than IPF, non-IPF IIPs and OB/BOOP.

### Example 21

### Collagen-Induced Arthritis (CIA) in a Mouse Model

### Relevance

Collagen-induced arthritis (CIA) in the mouse has proven to be a useful model of RA because it exhibits clinical and histopathologic features similar to those of the human disease and demonstrates many of the cellular and humoral immunity characteristics found in human RA (Cuzzocrea et al. Arthritis & Rheumatism, 52:940-950, 2005 and Devesa et al. Arthritis & Rheumatism, 52:3230-3238, 2005). Additionally, the recruitment and activation of neutrophils, macrophages and lymphocytes into joint tissues and the formation of pannus are hallmarks of the pathogenesis of both CIA and human RA (Cuzzocrea et al. Arthritis & Rheumatism, 52:940-950, 2005).

### Protocol

DBA/1J mice (9-wk-old) are housed in a controlled environment and are provided with access to standard rodent laboratory food and water. On day 1, the animals are treated with type II collagen (CII), injected intradermally at the base of the tail as a 100 uL emulsion containing 100 ug of CII and Freund's complete adjuvant (CFA), and with a second injection of CII on day 21. The development of arthritis in mice is evaluated daily starting on day 20 after the first intradermal injection, using a macroscopic scoring system as follows: 0=no signs of arthritis; 1=swelling and/or redness of the paw or 1 digit; 2=2 joints involved; 3=more than 2 joints involved; 4=severe arthritis of the entire paw and digits. An arthritis index for each mouse is calculated by addition the scores from the 4 individual paws. Clinical severity is also determined by quantitating the change in paw volume by plethysmometry (Cuzzocrea et al. Arthritis & Rheumatism, 52:940-950, 2005). Compounds of this invention are dosed via i.p administration twice a day at the dose of 1 mg/kg to 100 mg/kg of body weight starting from days 22 to 29 and are sacrificed on day 30 after CIA induction. A control group of animals receives i.p saline.

### Histological Examination

On day 35, animals are anesthetized and killed, and paws and knees are removed and fixed in 10% formalin. The paws are then trimmed, placed in decalcifying solution for 24 hours, embedded in paraffin and sectioned at 5 um, stained with hematoxylin and eosin and studied using light microscopy. Arthritis damage (histologic damage score) is evaluated and scored by an investigator who is blinded with regard to the treatment regimen. Morphologic features are scored as: 0=no damage, 1=edema, 2=presence of inflammatory cells, 3=bone resorption.

### Measurement of Cytokines

Hind paws are amputated above the ankle and homogenized in 1 mL of 10 mM HEPES buffer, pH 7.4, containing 0.32M sucrose, 100 mM EDTA, 1 mM dithiothreitol, 2 mM phenylmethylsulfonyl fluoride, and 100 mM leupeptin. After centrifugation at 1,200 g for 15 minutes at 4 C supernatants are removed and used for determination of cytokine levels, specifically TNF-α and IL-1β, by ELISA.

Quantification of Angiogenesis within the Joint

Endothelial cells are detected using a GSL-1 lectin immunohistochemical staining with the steptavidin-biotinperoxidase complex method. Knee joint slides are deparaffinized in xylene and dehydrated through serially diluted ethanol solutions down to distilled water. After blocking endogeneous peroxidase activity in blocking solutions for 1 hour, the slides are pretreated with blocking serum and then incubated with GSL-1 isolectin B4 for one hour at room temperature. Then, the slides are incubated with goat antibody to GSL-1 isolectin B4 for one hour, washed, and incubated with biotinylated rabbit antigoat immunoglobulins for 30 min in a moist chamber at room temperature. The samples are incubated with streptavidin-biotinperoxidase for 10 min using diaminobenzidine tetrahydrochloride as the chromogen. Between each step, the slides are washed three times for 5 min with TBS. They are then counterstained by incubation with hemalun for 40 sec and mounted with Glycergel. For each mouse, three non-serial sections from each knee are studied. For each section, five pictures are taken at low magnification, for example 400×. The area of the picture that is not in the synovium is subtracted from the total area. Any GSL-1 stained cell or group of cells with a lumen is considered as an individual vessel. Synovial vascular density is calculated as follows: each vessel in the synovium is counted and the number of vessels is divided by the synovium area. (Yin L et al. Mol Cancer Ther, 6: 1517-1525, 2007)

### Results

After day 30 after the first CII administration, the arthritis damage, edema, cellular influx, cytokine production, and degree of angiogenesis are measured and compared in the compound-treated mice vs. saline-treated mice. Improvements in at least one of the above-mentioned endpoints are observed.

### Example 22

### Mouse Model of TNBS-Induced Colitis as a Model of IBD

### Protocol

C57BL/10 male mice (6-8 weeks old) are used in studies of the acute form of TNBS-colitis. BALB/c female mice (8-10 weeks old) are used in studies of a chronic form of TNBS-colitis. TNBS (2,4,6-trinitrobenzene sulfonic acid) is a haptenation agent used to induce colitis.

Mice are lightly anesthetized with isoflurane and then administered a haptenating agent (either TNBS or oxazolone dissolved in ethanol) per rectum via a catheter equipped with a syringe; the catheter is then advanced into the rectum until the tip is 4 cm proximal to the anal verge at which time the haptenating agent is administered in a total volume of 150 μl. To ensure distribution of the haptenating agent within the entire colon and cecum, mice are held in a vertical position for 30 seconds after the intra-rectal injection. Control mice are administered an ethanol solution without haptenating agent using the same technique. 3 mg TNBS in 45% ethanol is used for studies of treatment of established acute TNBS-induced colitis and 1.5-2.5 mg TNBS (in increasing doses) in 45% ethanol is administered each week for studies of treatment of chronic TNBS-induced colitis.

### Inflammation

Compounds of this invention are administered to examine prevention of nascent TNBS-Colitis. Colitis is induced in C57BL/10 mice, by intra-rectal instillation of TNBS in ethanol as described above and then, 4 hours later compounds of this invention are administered by instillation or intra-peritoneal injection at a dose of 1 mg/kg to 100 mg/kg by body weight after TNBS administration and again on day 1 and day 2 after TNBS administration.

### Fibrosis

Compounds of this invention are administered to examine the prevention of development of colonic fibrosis in chronic TNBS-colitis. In this study, TNBS is administered by the intra-rectal route each week for 8 weeks to mice. On day 35 after initiation of TNBS administration, mice are assembled into weight-matched sub-groups for various types of treatment. Mice are treated with Rho Kinase inhibitor Compounds of Formula I either intra-rectally on days 37 and 44 or intra-peritoneally daily on days 37 to 39 and days 44 to 46 at a dose of 1 mg/kg to 100 mg/kg of body weight. A similar regimen is followed for mice treated with vehicle control.

### Histological Examination

Colons are fixed in 10% buffered formalin and embedded in paraffin. Paraffin-embedded colon sections are cut and then stained with H&E or by the Masson's trichrome method. For calculation of inflammation indices or for assessment of fibrosis in treated and control group of mice, the sections are read masked and evaluated according to a formerly published scoring system.

### Results

Following histological examination of the inflammation and fibrosis of the colon of control mice vs. mice treated with compounds of this invention, improvements in at least one of the above-mentioned endpoints is observed in the compound-treated mice.

### Example 23

### Murine Model of DSS-Induced Colitis

### Relevance

Dextran sulfate sodium (DSS)-induced colitis in mice shows reproducible morphological changes, which are very similar to those seen in patients with ulcerative colitis, or IBD (Hollenbach, E. et al. FASEB J; 13:1550-2, 2004).

### Protocol

Female BALB/c mice (6-8 weeks old) are used in studies of colitis. Mice are weighed and placed into groups randomly. Histological scoring and clinical assessments of colitis are performed in a masked fashion. The mice are adapted for 3 days following arrival after which colitis is induced by addition of 3% DSS (dextran sodium sulfate; Sigma) to normal drinking water for one week. After week one, DSS addition to water is stopped. Treatment with 200 μl 0.9% NaCl or compound of this invention at 1 mg/kg to 100 mg/kg body weight solution by intraperitoneal injection twice a day is administered beginning 60 hours after DSS treatment. Bowel tissue from untreated animals and animals treated with compound of this invention are evaluated for the degree of edema, mucosal injury, and infiltration of inflammatory cells into the colonic bowel. (Hollenbach, E. et al. FASEB J; 13:1550-2, 2004.)

### Clinical and Histological Analysis

Colitis score is calculated by assigning scores based on parameters from the disease activity index (DAI). The range varies from 0 (healthy) to 4 (maximal activity of colitis). On days 3, 5, 7, 10, and 13, mice (n=7 in each group per day) are euthanized by CO₂ inhalation. Immediately following, the colon is quickly removed, opened longitudinally, and cleared of fecal matter. Colon samples are either fixed in 4% buffered formalin or embedded in paraffin and 4-μm-thick serial step sections are stained with hematoxylin-eosin (HE). HE stained colon samples are histologically scored on a scale of 0 to 4 for inflammation, extent, regeneration, and crypt damage.

### Histological Disease Score

Histological parameters of experimentally induced colitis and the effects of compounds of this invention are evaluated. Treatment of mice with DSS produces a mild colitis after three days with multiple erosive lesions and inflammatory cell infiltrations. The impairment of the glandular architecture and the infiltration of macrophages, lymphocytes, and occasional eosinophils and neutrophils between day 3 and 7 are evaluated.

### Differential White Blood Cell Count

Blood, around 0.4 mL, is drawn intracardially and mixed with 50 μl of 0.5 M EDTA. Blood samples are subjected to differential blood cell count analysis, including Monocytes and peripheral granulocytes, after induction of colitis starting at day 5 through day 13.

### Results

After discontinuation of DSS, the resolution of inflammation, mucosal injury, and the degree of edema in the colonic bowel are measured and compared in the saline-treated mice vs. the mice treated with a compound of this invention. Improvements in at least one of the above-mentioned endpoints is observed.

### Example 24

### Animal Model of Neuropathic Pain

This example illustrates the efficacy of compounds in this invention in treatment of neuropathic pain in the mouse

### Protocol

The model is created as in Büyükaf [symbol] ar K et al. Eur J Pharmacol, 541 (1-2):49-52, 2006. Male balb/c mice are housed under standard conditions with access to water and mice chow ad libitum. In the hot plate test, the mice are administered vehicle (saline) or compound of Formula X (1-100 mg/kg, i.p. injection) and are then placed on a hot plate apparatus, which is thermostatically maintained at 55±1° C. Latency is the time elapsed in seconds until the mouse licks its forepaws. The latency is recorded in control and treatment groups with a cut-off time of 30 s.

In the abdominal constriction response test, the mice are administered vehicle (saline) or P2X7 receptor antagonist (1-10 mg/kg, i.p. injection). Thirty minutes later, 0.6% acetic acid is injected into the mice by the intraperitoneal route. Thereafter, each mouse is placed in an individual clear plastic observation chamber and the total number of writhes made by each mouse is counted for 15 min.

### Results

Latency on the hot plate and number of writhes are compared between control animals and those treated with compounds of Formula I. Latency is increased and number of writhes is decreased in those animals treated with compounds of I, demonstrating an antinociceptive effect.

### Example 25

### Animal Model Alzheimer's Disease Involving Aβ₄₂ Regulation

This example illustrates efficacy of compounds in this invention in treatment of Alzheimer's disease in mice

### Protocol

6-week old transgenic PDAPP mice are used in the experiments (Zhou Y et al. Science, 302: 1215-17, 2003). The animals are dosed by intracerebroventricular (ICV) injection. Mice are anesthetized and body temperature maintained at a consistent 37° C. The animals are placed in a stereotaxic apparatus with the incisor bar set at 3.3 mm below the interaural line. Small burr holes are made in the parietal bone to allow for the insertion of the injection cannula. All stereotaxic coordinates are determined based on the atlas of Paxinos and Watson. Brain levels of total Aβ and Aβ₄₂ are measured 28 hours after injection. Other parameters which could be measured include histological analysis of the plaque volume area as well as memory retention.

### Amyloid Extraction

Animals are euthanized and brain tissue is harvested. A total of 100 mg of brain tissue is homogenized in 1 mL of pure formic acid. Two microliters of the brain homogenate are used for the dot-blotting, or 50 μL are evaporated under nitrogen, solubilized in 50 μL of the SDS sample buffer (5% SDS, 20% glycerol, 2% β-mercaptoethanol, 150 mM Tris-HCl pH 6.8), and boiled 10 minutes before electrophoresis. A total of 10 μL (100 μg of protein) are loaded per well. Amyloid pathology is analyzed using electrophoresis adapted to the separation of small peptides. After the proteins are blotted on nitrocellulose membranes, the upper part is reacted with AD2 for the estimation of tau pathology. The lower part of the membranes is reacted first with ADA40 for the detection of Aβ 40 and then, after stripping, with 21F12 or ADA42 for the detection of Aβ₄₂ species.

### Plaque Volume Analysis

All animals are euthanized by a pentobarbital overdose and perfused with 4% paraformaldehyde. The brains are removed and coronal sections, 30 μm in thickness, are cut using a cryostat from the entire brain. Sections from the mouse brains are stained using appropriate antibodies and sections are examined under a light microscope; all consecutive sections throughout the plaque region are used to measure the plaque volume by an investigator blinded to the identity of the brains. Hemispheric areas for both sides of the tissue are measured to account for any brain swelling due to the stroke. Plaque volume is calculated in millimeters squared by multiplying the individual area measurements by the number of sections and by the distance between each section (Whitehead et al. Stroke, 38 (12): 3245-3250, 2007).

### Circular Platform Test

Memory and spatial learning behavioral skills are assessed using the Barnes circular platform pest. The behavioral test is divided into 3 phases: training, testing, and reacquisition phases. The time to reach the escape hole is measured as well as the number of errors (nose poked into wrong hole location). There are 3 recovery periods (7, 14, and 28 days after surgery), after which a single trial is performed with the hole at the initial training location (test). During the reacquisition phase, mice relearn the behavioral task (14 trials) with the hole location rotated by 135° (Whitehead et al. Stroke, 38 (12): 3245-3250, 2007).

Dosing of Compounds of this Invention

Compounds of this invention are dosed via ICV injection at the dose of 0.1 mg/kg to 20 mg/kg of body weight. A control group of animals receive the same volume of vehicle.

### Results

The ratio of Aβ/Aβ₄₂ levels in the brain, the plaque volume in the brain tissue and memory retention are measured and compared in the compound-treated mice vs. vehicle treated mice. An improvement in at least one of the above-mentioned endpoints is observed.

### Example 26

### Model of Experimental Autoimmune Encephalomyelitis in Mice

This assay of experimental autoimmune encyphalomyelitis (EAE) is a useful model of animal multiple sclerosis (MS) and is used to evaluate the effects of pharmacological agents on preventing or ameliorating the MS-like effects produced by the model.

### Protocol

The strain of SJL/J mice (6-7 weeks old) is immunized with a proteolipid protein (PLP) peptide (200 μg emulsified in complete Freund's adjuvant containing heat-killed mycobacterium tuberculosis) by injection subcutaneously in both sides of the rear flank. 200 ng of pertussis toxin is given i.p. at the time of immunization and 48 hr later. This protocol gives rise to signs and symptoms of animal MS. Mice are weighed and examined for clinical signs of EAE and are scored from 0 to 5 for severity of EAE (0=normal, 1=limp tail; 2=impaired rightening reflex; 3=partial hind limb paralysis; 4=total hind limb paralysis; 5=moribund or death). (Sun X., et al., J Neuroimmunol. 180:126-134. 2006). Compounds of this invention are administered i.p. at dose range of 1-100 mg/kg/day beginning 4 days before immunization and/or for 14-42 days following immunization. Control animals are given i.p. vehicle injections at the same time of dosing of compound.

Histopathology and immunohistochemistry are conducted on mice with experimental EAE following anesthesia and perfusion with PBS and 4% paraformaldehyde. Spinal cords are collected on various days (beginning at day 14 and up to day 70) after antigen immunization. Routine fixation, paraffin embedding, and hemotoxylin-eosin staining techniques are used to evaluate spinal cord pathology and for the infiltration of inflammatory cells as graded by CF45 immunostaining. Percentages of demyelinated areas per total white matter areas are examined by myelin basic protein (MBP) staining and areas of axonal loss per total white matter area are examined by neurofilament immunostaining. Axonal transaction is evaluated by amyloid precursor protein (APP) immunostaining and quantified by counting APP positive axons in a defined quarter of each section and calculated as positive axons per mm². Detailed protocol can be found in Sun X., et al., J Neuroimmunol. 180:126-134. 2006

### Results

The following outcomes are evaluated in the murine model of EAE: (1) clinical signs of EAE (scored 1-5); (2) reduction of inflammatory cell infiltration; (3) attenuated demyelination; and (4) acute axonal transaction. Improvements in at least one of these outcomes are seen with compounds of this invention compared with vehicle treatment.

### Example 27

### Efficacy of a Compound of Formula I or II in Reducing Inflammation in Rabbit Model of Meibomianitis, Blepharitis, and Conjunctivitis

Blepharitis is accompanied by increased inflammation in the eye lid and the surrounding tissue. The following assays demonstrates efficacy of a Compound of Formula I in decreasing this inflammation.

New Zealand white rabbits are anesthetized with ketamine (100 mg/kg) and xylazine (10 mg/kg). Meibomian gland duct orifices are closed by cautery in the right eyes of all rabbits as previously described (Gilbard J P, et al. “Tear film and ocular surface changes after meibomian gland orifice closure in the rabbit.” Opthalmology, 96:1180-1186, 1989). Animals are divided into four treatment groups (designated groups I, II, III, and IV): group I receives no treatment; group II receives vehicle only four times a day for five days each week; group III receives tetracycline hydrochloride 1% (w/v) (Sigma Chemical, St. Louis, Mo.) four times a day for five days each week; and group IV receives a Compound of Formula I or II (between 0.01 and 5.0%, w/v) four times a day for five days each week. Treatments begin at 8 weeks post-op and continue until 20 weeks.

All rabbits are sacrificed at 20 weeks postoperatively by overdose with pentobarbital. At the time of death, corneal epithelium is removed for measurement of corneal epithelial glycogen level as previously described (Friend J et al. Invest Opthalmol Vis Sci, 24:203-207, 1983; Sherwood M B et al. Opthalmology, 96:327-335, 1989). Conjunctival biopsies are then taken for counting of goblet cell density as previously described (Gilbard J P et al. Invest Opthalmol V is Sci, 28:225-228, 1987). Lower eyelids are then removed by sharp dissection and placed in one-half strength Karnovsky's fixative. The tissue is dehydrated through graded alcohols and embedded in methacrylate. Three micron sections are cut through the eyelids horizontally for light microscopy, and stained with alkaline giemsa.

Leukocytes are quantified in tissue sections using a method similar to that described by Sherwood et al. (Sherwood M B et al. Opthalmology, 96:327-335, 1989). For descriptive purposes, eyelid tissues are divided into three zones: 1) tarsal conjunctival epithelium, 2) underlying stroma, and 3) meibomian glands and adjacent tissue, including tarsal plate. Two separate sections, separated by a distance sufficient to provide two separate inflammatory cell populations, are examined for each eyelid. Leukocytes are identified as either neutrophils, eosinophils, basophils, or mast cells.

Twenty weeks after meibomian gland orifice closure, untreated rabbits have a significant increase in eyelid tissue mast cells, eosinophils, neutrophils and basophils relative to unoperated controls. Mast cells are not seen in the conjunctival epithelium of normal eyes nor after meibomian gland orifice closure. With this exception, all leukocyte types increase in all three tissue zones. Treatment with a Compound of Formula I decreases the number of leukocytes in the tissue when compared with vehicle-treated animals.

### Example 28

### Efficacy of a Compound of Formula I or II in Reducing Inflammation in Model of Lacrimal Gland Inflammation-Induced Dry Eye in Rabbits

### Protocol

The rabbit model of lacrimal gland inflammation-induced dry eye is used as an animal model of human dry eye disease. Rabbit lacrimal glands are injected with the T-cell mitogen Concanavalin A (Con A) to induce the conditions of dry eye. Measurements of inflammation, tear function, and corneal epithelial cell integrity are subsequently assessed as markers of efficacy. Matrix metalloproteinase-9 (MMP-9) and pro-inflammatory cytokines are quantified in tissue extracts. Tear function is monitored by measuring tear fluorescein clearance and tear breakup time (TBUT). Corneal epithelial cell integrity is determined by quantifying the uptake of methylene blue dye following the exposure of rabbits to a low humidity environment.

The compounds of Formula I or II in the concentration range 0.01-5% w/v or vehicle control is administered as a topical ophthalmic formulation with a positive displacement pipette in a volume of 30 μl to rabbits randomly assigned into treatment groups and dosed topically 4 times per day (QID) at various times during (prophylactic) or after (therapeutic) lacrimal gland injection.

### Results

Improvement in tear function and/or reduction of ocular surface injury or inflammation is observed in Compound-treated animals compared with vehicle-treated animals.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

What is claimed:

1. A method for preventing or treating diseases associated with inflammation or inflammatory conditions, neurodegenerative diseases, or pain in a mammal, comprising:
identifying a mammal in need thereof, and
administering to the mammal an effective amount of a compound of Formula VIa, VIb, VIIa, or VIIb, or a pharmaceutically acceptable salt, tautomer, hydrate, or solvate thereof, wherein said amount is effective to inhibit inflammation, neurodegenerative diseases, or pain; 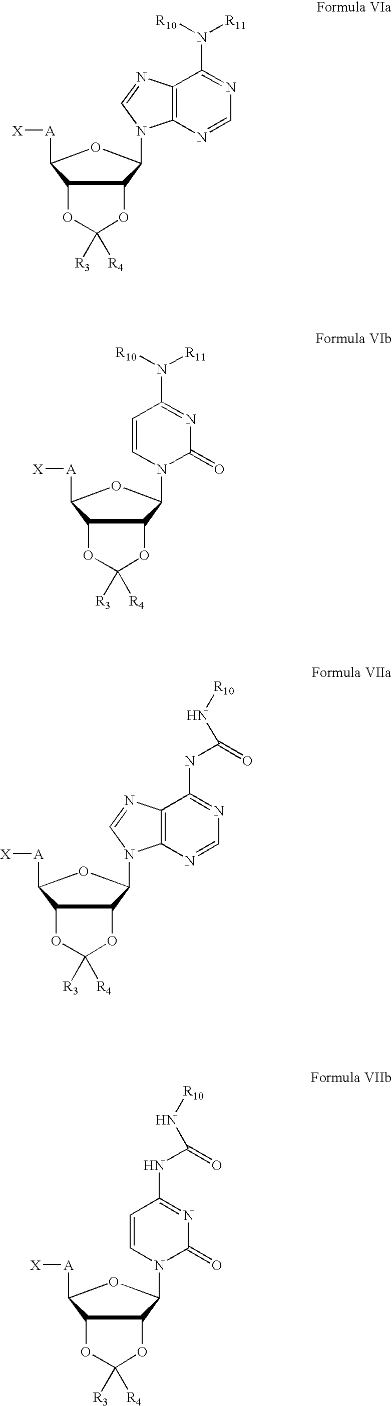
wherein X is a covalently bound substituent selected from the group —R₁, —OR₁, —COOR₁, —SR₁, —SO₃H, —S(O₂)NR₁R₂, —NR₁(CO)R₂, —NR₁(SO₂)R₂, —NR₁(SO₂)NR₁R₂, —(CO)NR₁R₂, —NH(CO)NR₁R₂, —NR₁(NH)NR₁R₂, —NR₁(CO)OR₂, —O(CO)NR₁R₂, —NR₁R₂, —OP(O)(OH)₂, and —CH₂—P(O)(OH)₂, where R₁ and R₂ are independently H, alkyl, alkenyl, alkynyl, alkoxy, aryl, arylalkyl, arylalkenyl, arylalkynyl, aryloxy, cycloalkyl, cycloalkenyl, cycloalkylalkyl, heteroaryl, heteroarylalkyl, heteroarylalkenyl, heteroarylalkynyl, or heterocycle;
A is CH₂, CH═CH, or absent; and for all moieties defined by A-X, the atom that is directly attached to the 4′ carbon of a ribose ring is not N, O, or S;
R₃ and R₄ are independently H, alkyl, alkenyl, alkynyl, cycloalkyl, cycloalkenyl, aryl, arylalkyl, arylalkenyl, arylalkynyl, with or without substitution; or
R₃ and R₄ are joined together to form a homocyclic or heterocyclic ring composed of 3 to 8 atoms;
R₁₀ and R₁₁ are independently H, alkyl, alkenyl, alkynyl, cycloalkyl, cycloalkenyl, aryl, arylalkyl, arylalkenyl, arylalkynyl, cycloalkylalkyl, heteroaryl, heteroarylalkyl, heteroarylalkenyl, heteroarylalkynyl, or heterocycle with or without substitution; or
when neither R₁₀ and R₁₁ is H, R₁₀ and R₁₁ are optionally joined together by a saturated or unsaturated bond, forming a ring of from 3 to 8 atoms, with or without substituents, unsaturation, or heteroatoms.

2. The method according to claim 1, wherein said disease associated with inflammation or inflammatory conditions is a pulmonary disease.

3. The method according to claim 2, wherein said pulmonary disease is asthma, chronic obstructive pulmonary disease, respiratory tract illness caused by respiratory syncytial virus, idiopathic pulmonary fibrosis, acute respiratory distress syndrome and ventilator induced lung injury, cystic fibrosis, bronchiectasis, alpha-1-antitrypsin deficiency, rhinitis, rhinosinusitis, primary ciliary dyskinesia, pneumonia, bronchiolitis caused by agents other than respiratory syncytial virus, OB/BOOP due to lung transplantation of HSCT, non IPF-IIPs, or OB/BOOP.

4. The method according to claim 1, wherein said disease associated with inflammation or inflammatory conditions is inflammatory bowel disease or rheumatoid arthritis.

5. The method according to claim 1, wherein said disease associated with inflammation or inflammatory conditions is dry eye disease or blepharitis.

6. The method according to claim 1, wherein said pain is neuropathic pain.

7. The method according to claim 1, wherein said neurodegenerative disease is multiple sclerosis or Alzheimer's disease.

8. The method according to claim 1, wherein said compound is a compound of Formula VIa.

9. The method according to claim 1, wherein said compound is a compound of Formula VIb.

10. The method according to claim 1, wherein said compound is a compound of Formula VIIa.

11. The method according to claim 1, wherein said compound is a compound of Formula VIIb.

12. The method according to claim 1, wherein R₃ and R₄ are joined to form a cycloalkyl ring or an aracycloalkyl ring.

13. The method according to claim 1, wherein R₃ and R₄ are independently alkyl, alkenyl, alkynyl, cycloalkyl, cycloalkenyl, aryl, arylalkyl, arylalkenyl, or arylalkynyl, with or without substitution.

14. The method according to claim 1, wherein A is CH₂, X is OH, NR₁(CO)R₂, —O(CO)NR₁R₂, —NH(CO)NR₁R₂, or —NR₁R₂, such that the combined moiety X-A is respectively an alcohol, an amide, a carbamate, a urea, or an amine.

15. The method according to claim 1, wherein A is a carbonyl, X is OH, or —NR₁R₂, such that the combined moiety A-X is respectively a carboxylic acid or an amide.

16. The method according to claim 1, wherein said compound is a compound of Formula VIa or VIb, and R₁₀ and R₁₁ are independently H, cycloalkyl, cycloalkylalkyl, aryl, or arylalkyl, provided both R₁₀ and R₁₁ are not H.

17. The method according to claim 1, wherein said compound is a compound of Formula VIIa or VIIb, and R₁₀ is cycloalkyl, cycloalkylalkyl, aryl, or arylalkyl.

18. The method according to claim 1, wherein said compound is 6-(adamantylurea)adenosine 2′,3′-cyclohexyl ketal (38), 6-(cyclohexylurea)adenosine 2′,3′-cyclohexyl ketal (39), 6-(cyclohexylurea)adenosine 2′,3′-cyclohexyl ketal 5′-ethyl carboxamide (43), and 6-(cyclohexylurea)adenosine 2′,3′-cyclohexyl ketal 5′-(2-hydroxy)ethyl carboxamide (44).

19. A method for inhibiting the release of interleukin-1β in a mammal comprising:
administering to a mammal in need thereof an effective amount of a compound of Formula VIa, VIb, VIIa, or VIIb, or a pharmaceutically acceptable salt, tautomer, hydrate, or solvate thereof, wherein said amount is effective to inhibit the release of interleukin-1β; 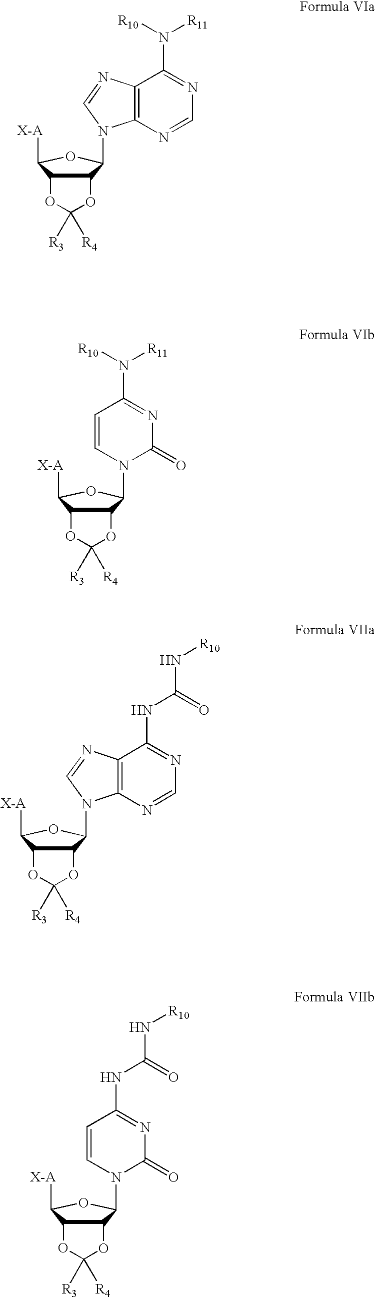
wherein:
X is a covalently bound substituent selected from the group —R₁, —OR₁, —COOR₁, —SR₁, —SO₃H, —S(O₂)NR₁R₂, —NR₁(CO)R₂, —NR₁(SO₂)R₂, —NR₁(SO₂)NR₁R₂, —(CO)NR₁R₂, —NH(CO)NR₁R₂, —NR₁(NH)NR₁R₂, —NR₁(CO)OR₂, —O(CO)NR₁R₂, —NR₁R₂, —OP(O)(OH)₂, and —CH₂—P(O)(OH)₂, where R₁ and R₂ are independently H, alkyl, alkenyl, alkynyl, alkoxy, aryl, arylalkyl, arylalkenyl, arylalkynyl, aryloxy, cycloalkyl, cycloalkenyl, cycloalkylalkyl, heteroaryl, heteroarylalkyl, heteroarylalkenyl, heteroarylalkynyl, or heterocycle;
A is CH₂, CH═CH, or absent; and for all moieties defined by A-X, the atom that is directly attached to the 4′ carbon of a ribose ring is not N, O, or S;
R₃ and R₄ are independently H, alkyl, alkenyl, alkynyl, cycloalkyl, cycloalkenyl, aryl, arylalkyl, arylalkenyl, arylalkynyl, with or without substitution; or
R₃ and R₄ are joined together to form a homocyclic or heterocyclic ring composed of 3 to 8 atoms;
R₁₀ and R₁₁ are independently H, alkyl, alkenyl, alkynyl, cycloalkyl, cycloalkenyl, aryl, arylalkyl, arylalkenyl, arylalkynyl, cycloalkylalkyl, heteroaryl, heteroarylalkyl, heteroarylalkenyl, heteroarylalkynyl, or heterocycle with or without substitution; or
when neither R₁₀ and R₁₁ is H, R₁₀ and R₁₁ are optionally joined together by a saturated or unsaturated bond, forming a ring of from 3 to 8 atoms, with or without substituents, unsaturation, or heteroatoms.

20. A compound selected from the group consisting of Compounds 1-56: 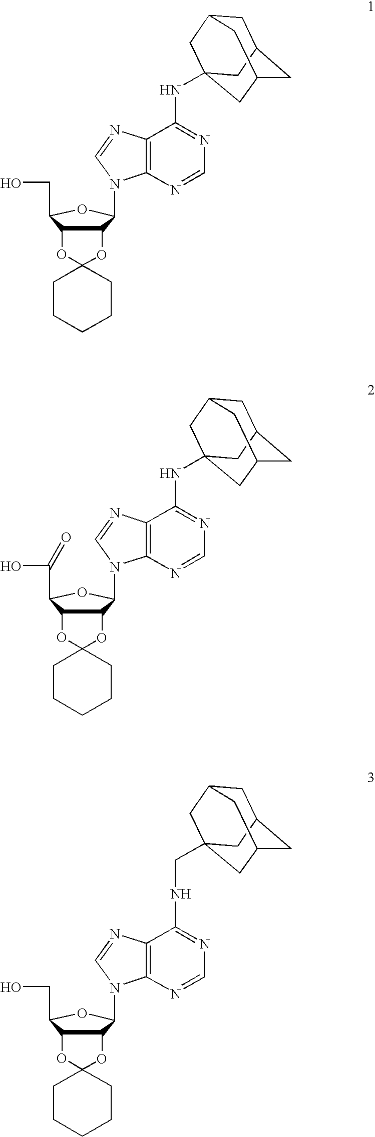 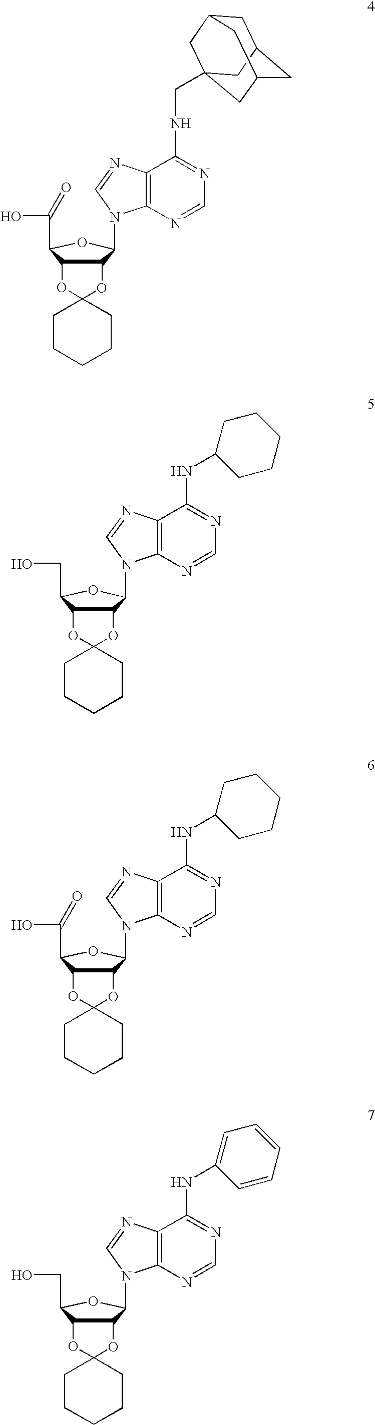 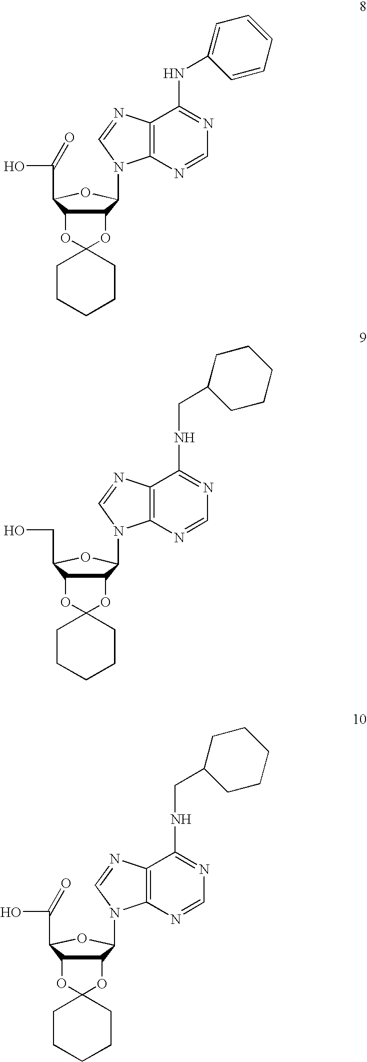 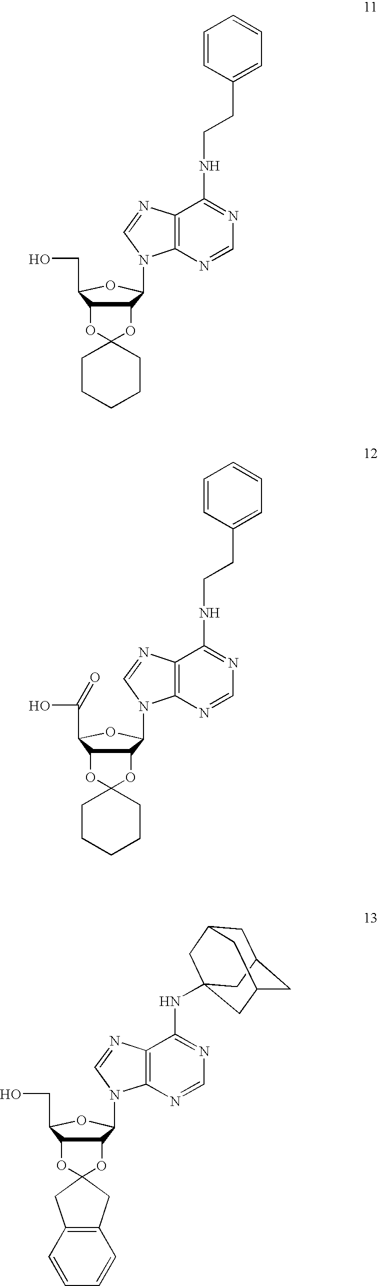 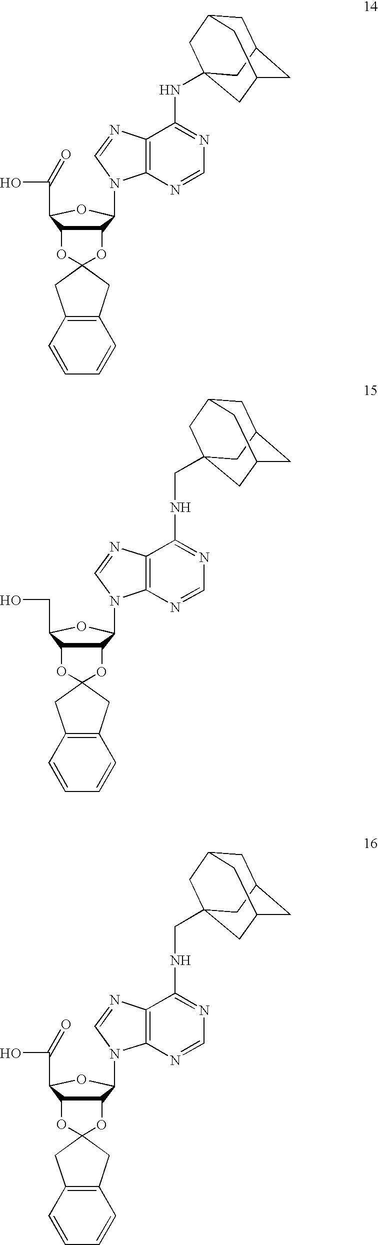 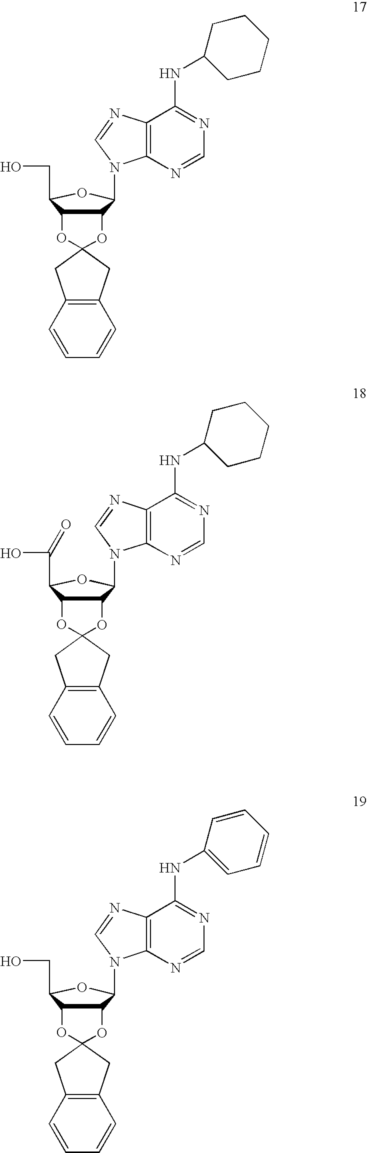 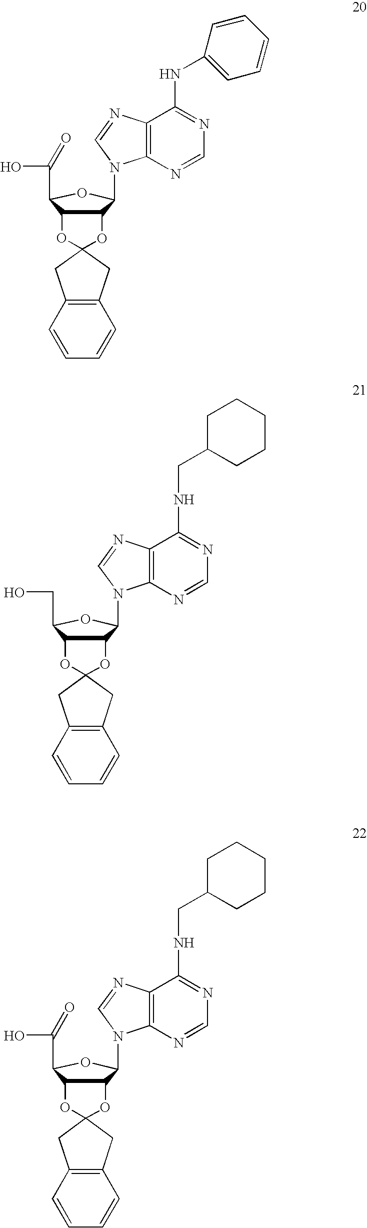 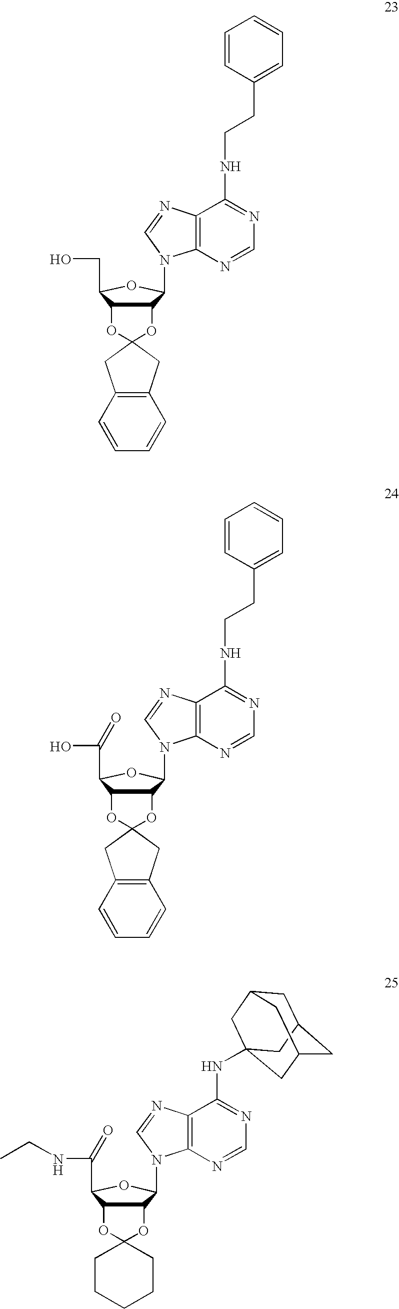 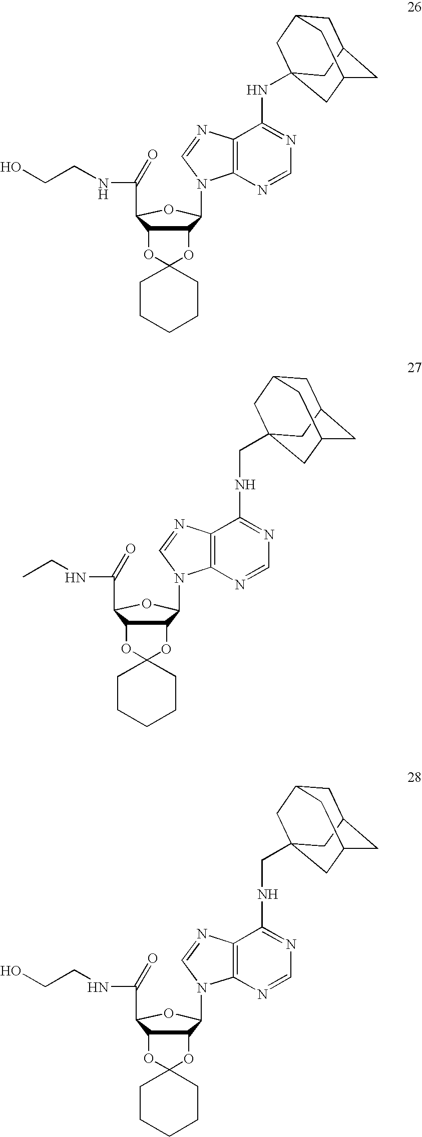 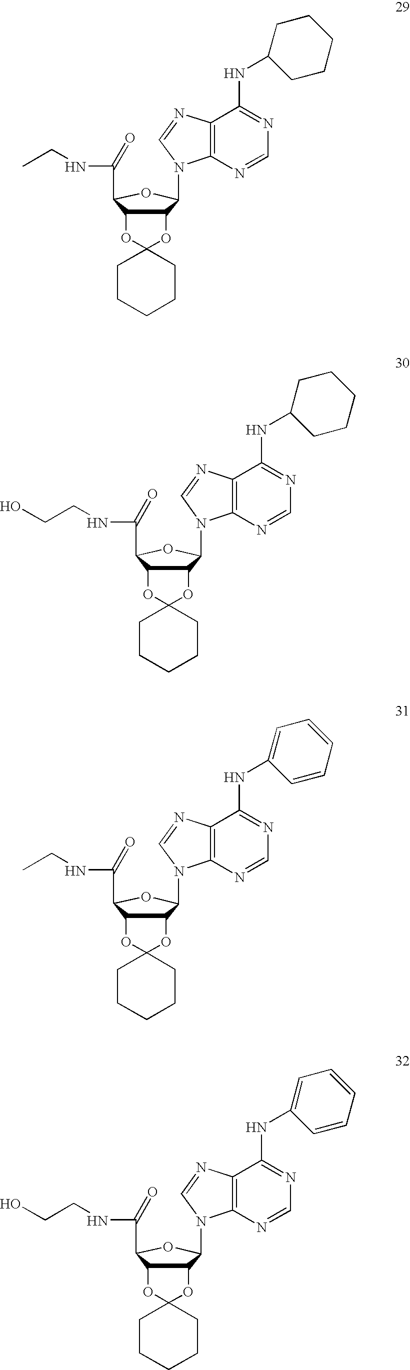 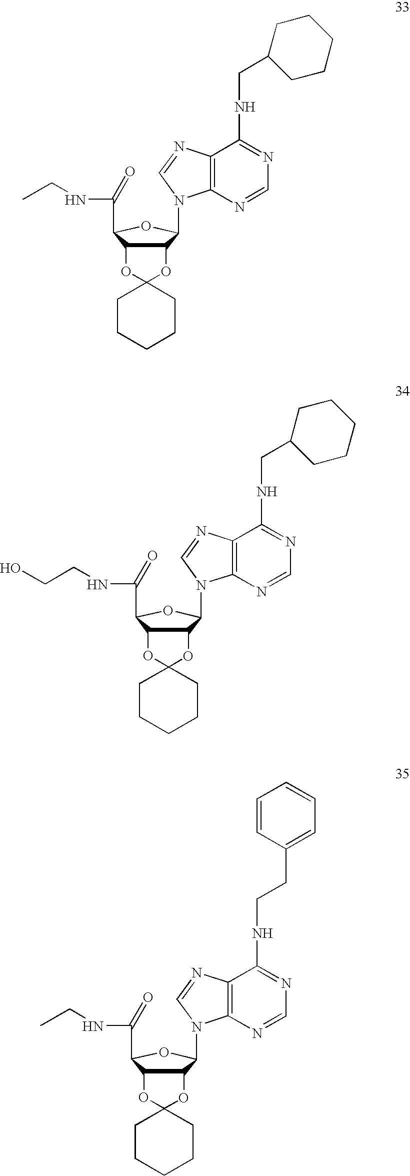 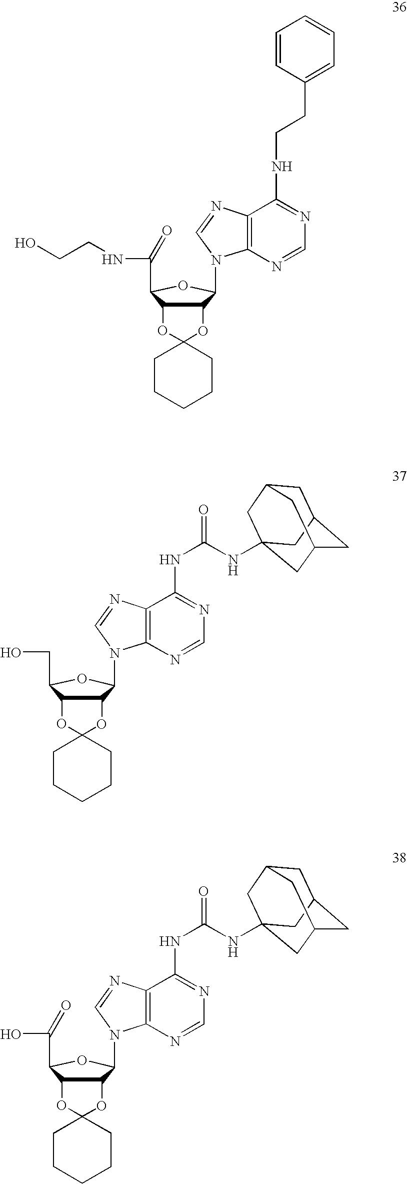 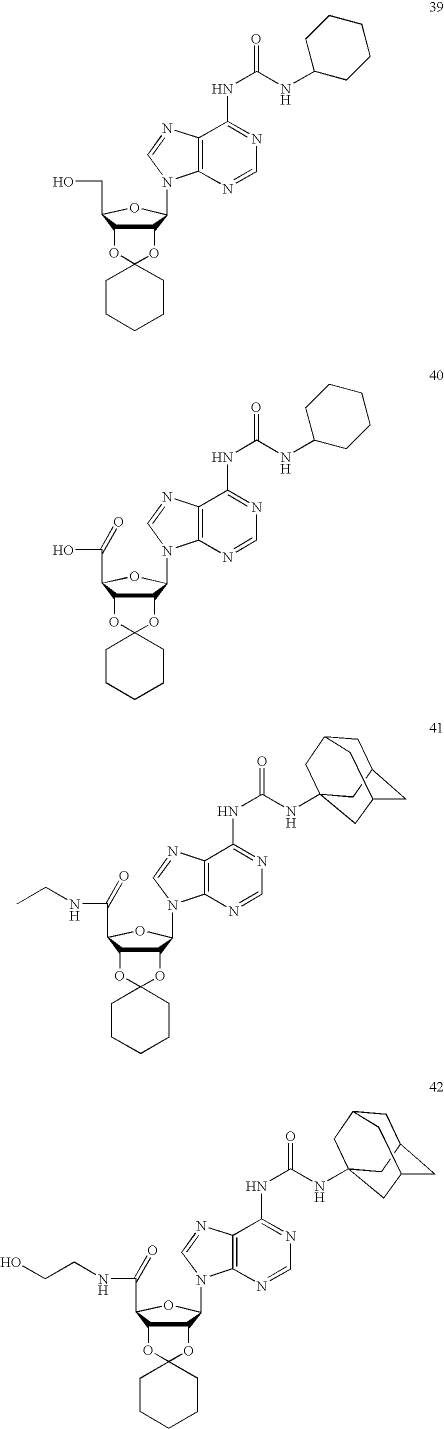 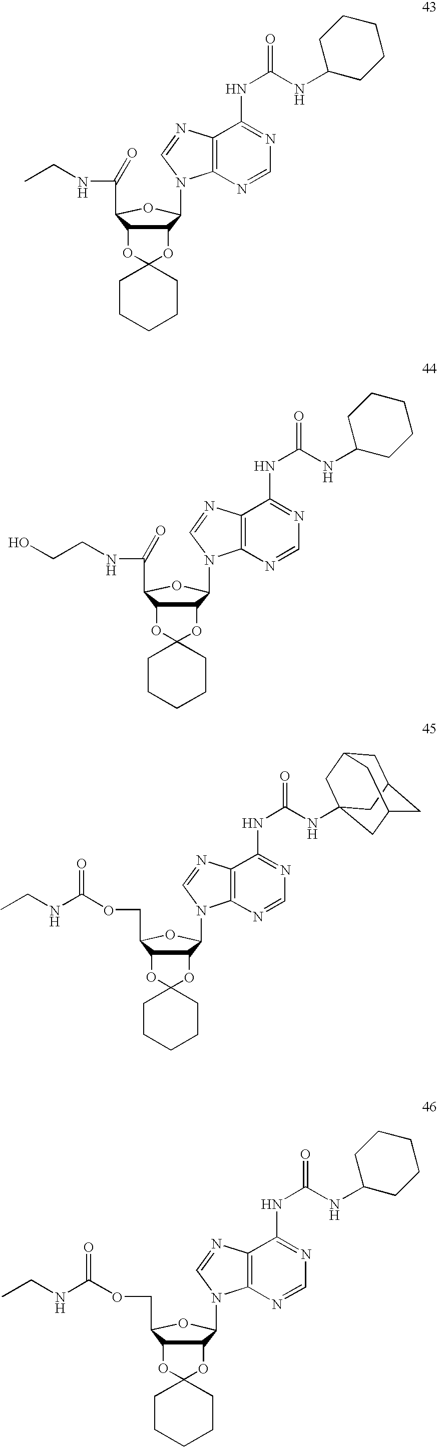 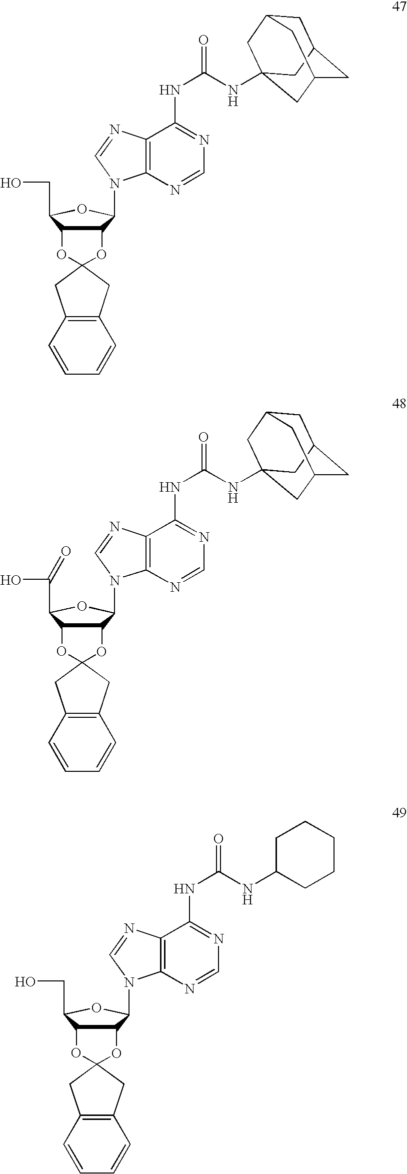 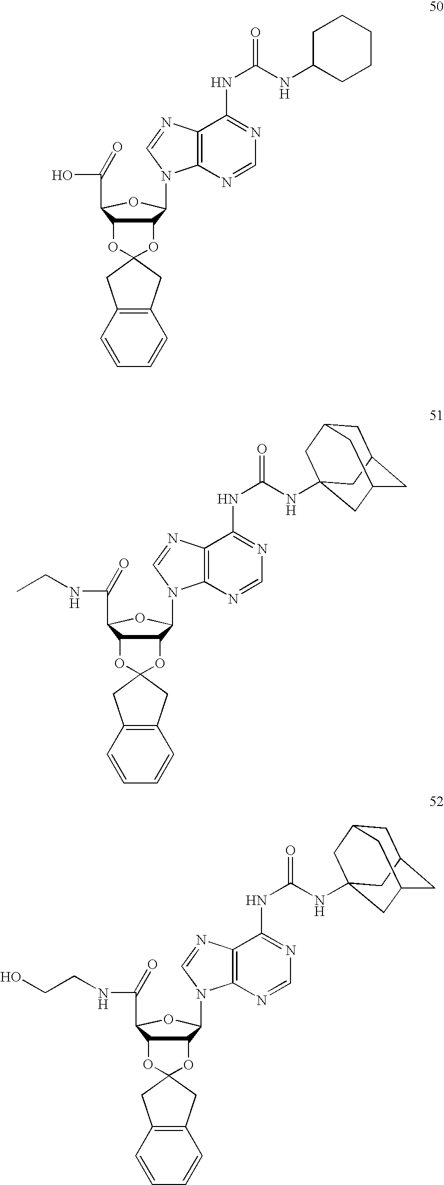 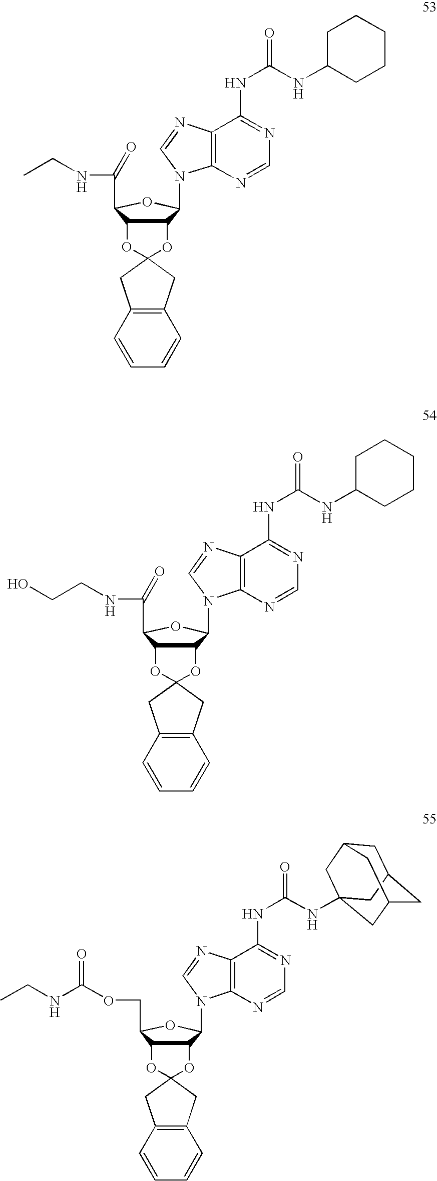 [chemical structure]
